(19) United States
(12) Patent Application Publication
(10) Pub. No.: US 2024/0285773 A1
(43) Pub. Date: Aug. 29, 2024

(54) METHODS OF TREATING MYELODYSPLASTIC SYNDROME AND MONITORING THE TREATMENT

(71) Applicant: Geron Corporation, Foster City, CA (US)

(72) Inventors: Faye Feller, Parsippany, NJ (US); Fei Huang, Parsippany, NJ (US)

(21) Appl. No.: 18/394,773

(22) Filed: Dec. 22, 2023

Related U.S. Application Data
(60) Provisional application No. 63/606,256, filed on Dec. 5, 2023.
(60) Provisional application No. 63/520,538, filed on Aug. 18, 2023.
(60) Provisional application No. 63/505,918, filed on Jun. 2, 2023.
(60) Provisional application No. 63/503,896, filed on May 23, 2023.
(60) Provisional application No. 63/448,638, filed on Feb. 27, 2023.
(60) Provisional application No. 63/444,789, filed on Feb. 10, 2023.
(60) Provisional application No. 63/436,831, filed on Jan. 3, 2023.

(51) Int. Cl. A61K 47/54; A61P 35/00

(52) U.S. Cl. CPC A61K 47/549; A61K 47/543; A61K 47/548; A61P 35/00

(57) ABSTRACT

Methods of monitoring therapeutic efficacy in a subject with myelodysplastic syndrome (MDS) are provided. Also provided is a method of identifying a subject with MDS for treatment with a telomerase inhibitor, and methods of treating MDS. The methods include administering to the subject a telomerase inhibitor and assessing variant allele frequency (VAF) for one or more of the following genes: SF3B1, TET2, DNMT3A, ASXL1, and CUX1 in a biological sample obtained from the subject after administration of the telomerase inhibitor. In some cases, a 25% or more reduction in VAF identifies a subject who has an increased likelihood of benefiting from treatment with a telomerase inhibitor. In some instances, the telomerase inhibitor is imetelstat or imetelstat sodium.

### CROSS-REFERENCE TO RELATED APPLICATIONS

This application claims priority to U.S. Provisional Application No. 63/436,831, filed Jan. 3, 2023, U.S. Provisional Application No. 63/444,789, filed Feb. 10, 2023, U.S. Provisional Application No. 63/448,638, filed Feb. 27, 2023, U.S. Provisional Application No. 63/503,896, filed May 23, 2023, U.S. Provisional Application No. 63/505,918, filed Jun. 2, 2023, U.S. Provisional Application No. 63/520,538, filed Aug. 18, 2023, and U.S. Provisional Application No. 63/606,256, filed Dec. 5, 2023, the disclosures of which are incorporated herein by reference in their entireties.

### INCORPORATION-BY-REFERENCE OF MATERIAL ELECTRONICALLY SUBMITTED

A Sequence Listing is provided herewith as a Sequence Listing XML, “GERN-200_SEQ_LIST” created on Mar. 5, 2024 and having a size of 2,048 bytes. The contents of the Sequence Listing XML are incorporated by reference herein in their entirety.

### INTRODUCTION

Myelodysplastic syndromes (MDS) are a group of symptoms that includes cancer of the blood and bone marrow. They also include diseases such as refractory anemia, refractory anemia with excess blasts, refractory cytopenia with multilineage dysplasia, refractory cytopenia with unilineage dysplasia, and chronic myelomonocytic leukemia. The MDS are a collection of hematological medical conditions that involve ineffective production of the myeloid class of blood cells. In MDS, the immature blood stem cells (blasts) do not become healthy red blood cells, white blood cells, or platelets. The blasts die in the bone marrow or soon after traveling to the blood leaving less room for healthy white cells, red cells, and/or platelets to form in the bone marrow.

MDS primarily affects the elderly and is characterized by anemia and other cytopenias and a high risk of leukemic transformation (Cheson et al., Blood 2006; 108:419-425). In clinical practice, MDS are suspected when an otherwise unexplained anemia is associated with other cytopenias, increased mean corpuscular volume, or increased red cell distribution width. Diagnosis involves bone marrow examination and cytogenetic studies. The bone marrow is typically hyperproliferative. Diagnosis is based on demonstration of erythroid, granulocyte, or megakaryocyte dysplasia in 10% or more of informative cells (Vardiman, et al., Blood 2009; 114(5):937-951). MDS may progress over time. For example, patients with MDS often develop severe anemia and require frequent blood transfusions. Bleeding and risk of infections also occur due to low or dysfunctional platelets and neutrophils, respectively. In some cases, the disease worsens, and the patient develops cytopenias (low blood counts) caused by progressive bone marrow failure. In other cases, the disease transforms into acute myelogenous leukemia (AML). If the overall percentage of bone marrow myeloblasts rises above a particular cutoff (20% for World Health Organization (WHO) and 30% for French-American-British (FAB) subtypes), then transformation to AML is said to have occurred. Limited treatment options exist for patients with lower risk MDS who are relapsed or refractory to erythropoiesis-stimulating agents (ESA).

The standard prognostic tool for assessing MDS is the International Prognostic Scoring System (IPSS), which classifies patients into low, intermediate-1, intermediate-2, and high-risk categories based on several prognostic variables including bone marrow blasts, cytogenetics, and presence of cytopenias. The median survival for these four groups has been estimated at 5.7, 3.5, 1.2, and 0.4 years, respectively. The median times for 25% of patients in these groups to develop AML were 9.4, 3.3, 1.1 and 0.2 years, respectively (Greenberg et al., Blood 1997; 89(6):2079-2088). Patients with low and intermediate-1 risk MDS may be referred to as having “lower-risk” disease, whereas those with intermediate-2 and high risk MDS may be referred to as patients with “higher-risk” disease.

In patients aged ≥70 years in Western countries, the incidence of MDS is conservatively estimated approximately at 30 to 40 cases per 100,000 population per year. Due to an aging population, the number of cases of MDS is expected to escalate. Despite the reduced rate of leukemic transformation of lower-risk patients, most patients are affected by anemia and anemia-related symptoms with profound effects on patient-reported outcomes (Almeida et al., Leukemia Res. 2017; 52:50-57). Many anemic patients with MDS eventually develop dependence on red blood cell (“RBC”) transfusions (“transfusion dependence”); evidence suggests that iron overload resulting from chronic RBC transfusion may be a contributing factor in the overall morbidity of the disease (Malcovati et al., J Clin Oncol 2005; 23:7594-7603; Malcovati et al., Haematologica 2006; 91:1588-1590; Steensma D P., Mayo Clinic Proc. 2015; 90(7):969-983). Analysis of retrospective data from 426 patients diagnosed with MDS according to WHO criteria in Italy between 1992 and 2004 showed that a transfusion requirement of 2 units per month reduces the life expectancy of a patient with MDS by approximately 50% (Malcovati et al., Haematologica 2006).

The treatment strategy for MDS is largely based on the IPSS score. In patients classified as IPSS intermediate-2 or high risk (higher-risk MDS), with median survival if untreated of only about 12 months, the treatment goal is modifying the disease course, avoiding progression to AML, and extending survival. In patients classified as IPSS low or intermediate-1 risk (lower-risk MDS), survival is longer, but many patients die from causes other than MDS. Treatment of these patients mainly aims to ameliorate the consequences of cytopenias and transfusions and improve quality of life (Ades et al., Lancet 2014; 383(9936): 2239-2252).

For patients with lower-risk non-del(5q) MDS, first-line treatment of anemia often involves the use of ESAs or other hematopoietic growth factors. High-dose ESAs (e.g. epoetin alfa), with or without granulocyte colony-stimulating factors, have yielded erythroid response rates in the range of 30% to 50% and of median duration 2 years (id.). Key favorable prognostic factors for response to ESAs are low or absent RBC transfusion requirement (<2 packed red blood cell units/month) and low serum erythropoietin level (≤500 units/L) (Hellstrom-Lindberg et al., Br J Haematol. 2003; 120(6):1037-1046). Studies have shown that ESAs have no effect on the risk of progression to higher-risk MDS and AML, and strongly suggest that they may even improve survival in lower-risk MDS compared with RBC transfusion alone (Garcia-Manero et al., J Clin Oncol. 2011; 29(5):516-523). In the absence of concomitant progression to higher-risk MDS or AML, patients, who had primary refractoriness to ESA or relapsed within 6 months of response achievement, were observed to have a relatively high risk of AML transformation (23.1%) and short survival (median 3 years), whereas patients, who responded to treatment and relapsed beyond 6 months, had a more favorable outcome after failure with a 9% AML risk at 7 years and a median overall survival of 4.5 years (Kelaidi et al., Leukemia 2013; 27(6):1283-1290).

There is no approved therapy in the United States for patients with lower-risk non-del(5q) MDS who are not responsive to ESA; and treatment options after ESA failure are limited. Most patients with lower-risk MDS will eventually require long-term RBC transfusion, which is often accompanied by iron overload (Ades et al., Lancet 2014; Fenaux et al., Blood 2013; 121:4280-4286; Steensma et al., Mayo Clinic Proc. 2015). Life expectancy for patients with MDS has been shown to be inversely related to RBC transfusion burden (Malcovati et al., Haematologica 2006). Patients with chronic anemia despite frequent RBC transfusions may be at risk for associated morbidities (e.g. cardiac failure, falls, fatigue) and lower quality of life (Crawford et al., Cancer 2002; 95:888-895).

Hypomethylating agents (HMA) (e.g. azacitidine and decitabine) have been approved as treatments for all French-American-British (FAB) subtypes, which includes some lower-risk MDS patients. While these drugs reduce transfusion requirements in higher-risk MDS patients, evidence for improvement of long-term outcomes for lower-risk patients who receive HMAs after ESA failure is absent. In a retrospective study of 1,698 patients with non-del(5q) lower-risk MDS treated with ESAs, patients receiving subsequent treatment with HMAs (n=194) after ESA failure did not experience significant improvement in 5-year overall survival (Park et al., J Clin Oncol. 2017; 35(14):1591-1597). According to other reports, in cohorts of patients with lower-risk MDS who are transfusion dependent after ESA failure, azacitidine induces RBC transfusion independence (“RBC-TI”) in approximately 14% to 33% of patients (Fili et al., Clin Cancer Res. 2013; 19:3297-3308; Thepot et al., Haematologica. 2016; 101:918-925; Tobiasson et al., Blood Cancer J. 2014: 4, e189). In view of the limited benefit and observed toxicities (neutropenia, infection), azacitidine cannot be recommended as treatment for these patients (Tobiasson et al., Blood Cancer J. 2014).

The del(5q) chromosomal abnormality is observed in 10% to 15% of patients with MDS and is associated with a favorable prognosis (Oliva et al., Ann Hematol. 2013; 92(1):25-32). Treatment with lenalidomide results in transfusion independence for approximately two-thirds of such patients (Ades et al., Lancet 2014; Fenaux et al., Blood 2013; 121(21):4280-4286). In a Phase 3 study, median duration of transfusion independence (“TI”) was not reached (median follow-up, 1.55 years) (Fenaux et al., Blood 2011; 118(14):3765-3776). Myelosuppression was the most frequently reported Grade 3 or 4 toxicity, and close monitoring of blood counts is required in the first weeks of lenalidomide therapy (id.).

Lenalidomide has also been studied as a treatment for transfusion-dependent non-del(5q) MDS, which represent 85% to 90% of the MDS population. The majority of these subjects do not respond to lenalidomide. Hematologic toxicity (i.e., neutropenia, and thrombocytopenia) was milder than in patients with del(5q) MDS (Loiseau et al., Exp Hematol. 2015; 43(8):661-72). Like HMAs, treatment with lenalidomide following ESA failure has not been shown to significantly improve overall survival when used to treat lower-risk non-del(5q) MDS patients (Park et al., J Clin Oncol. 2017).

While immunosuppressive therapy is a treatment option for certain lower-risk non-del(5q) patients, no significant effect on transformation-free survival was observed; and adverse events, including hematologic toxicity and associated severe adverse events, such as hemorrhage and infections, have been reported (Almeida et al., Leukemia Res. 2017). Allogeneic stem cell transplantation is typically reserved for medically fit higher-risk MDS patients, but may be considered an option for select lower-risk patients, such as those aged <60 to 70 years with IPSS intermediate-1-risk MDS, poor-risk cytogenetics, or persistent blast elevation, if alternative therapeutic options are ineffective (id.).

### SUMMARY

Methods of treating MDS in a subject and methods of monitoring the treatment are provided. Also provided is a method of identifying a subject with MDS suitable for treatment with a telomerase inhibitor, and methods of treating MDS. The subject methods can include administering to the subject an effective amount of a telomerase inhibitor and assessing the variant allele frequency (VAF) in a biological sample obtained from the subject for one or more of the following genes: SF3B1, TET2, DNMT3A and ASXL1. In some cases, a 25% or more reduction in the VAF for one or more of the following genes: SF3B1, TET2, DNMT3A and ASXL1, identifies a subject who has an increased likelihood of benefiting from treatment with a telomerase inhibitor. The subject methods can include administering to the subject an effective amount of a telomerase inhibitor and assessing the VAF in a biological sample obtained from the subject for one or more of the following genes: SF3B1, TET2, DNMT3A, CUX1 and ASXL1. In some cases, a 25% or more reduction in the VAF for one or more of the following genes: SF3B1, TET2, DNMT3A, CUX1 and ASXL1, identifies a subject who has an increased likelihood of benefiting from treatment with a telomerase inhibitor. The subject can be naive to treatment with a HMA, lenalidomide, or both. In some cases, the subject is classified as having low or intermediate-1 IPSS risk MDS and/or MDS relapsed or refractory to ESA. In certain cases, the subject is non-del5q. In some instances, the telomerase inhibitor is imetelstat or imetelstat sodium.

Methods of treating MDS in a subject with a telomerase inhibitor are provided. Particularly, a treatment with a telomerase inhibitor is continued if 25% or more reduction in the VAF for one or more of the following genes: SF3B1, TET2, DNMT3A and ASXL1 is observed in the subject after administration of a telomerase inhibitor. Particularly, a treatment with a telomerase inhibitor is continued if 25% or more reduction in the VAF for one or more of the following genes: SF3B1, TET2, DNMT3A, CUX1 and ASXL1 is observed in the subject after administration of a telomerase inhibitor. The telomerase inhibitor can be imetelstat or imetelstat sodium. The subject can be naive to treatment with a HMA, lenalidomide, or both. In some cases, the subject is classified as having low or intermediate-1 IPSS risk MDS and/or MDS relapsed or refractory to ESA. In certain cases, the subject is non-del5q. In some cases, the subject is classified as IPSS intermediate and poor cytogenetic risk.

In some cases, methods of treating MDS in a subject with a telomerase inhibitor, such as imetelstat, comprise administering to the subject a telomerase inhibitor, such as imetelstat and continuing administering to the subject a telomerase inhibitor, such as imetelstat because, after administration of the telomerase inhibitor, such as imetelstat, 25% or more reduction in the VAF was observed for one or more of the following genes: SF3B1, TET2, DNMT3A and ASXL1. The telomerase inhibitor can be imetelstat or imetelstat sodium. The subject can be naive to treatment with a HMA, lenalidomide, or both. In some cases, the subject is classified as having low or intermediate-1 IPSS risk MDS and/or MDS relapsed or refractory to ESA. In certain cases, the subject is non-del5q. In some cases, the subject is classified as IPSS intermediate and poor cytogenetic risk.

In some cases, methods of treating MDS in a subject with a telomerase inhibitor, such as imetelstat, comprise administering to the subject a telomerase inhibitor, such as imetelstat and continuing administering to the subject a telomerase inhibitor, such as imetelstat because, after administration of the telomerase inhibitor, such as imetelstat, 25% or more reduction in the VAF was observed for one or more of the following genes: SF3B1, TET2, DNMT3A, CUX1, and ASXL1. The telomerase inhibitor can be imetelstat or imetelstat sodium. The subject can be naive to treatment with a HMA, lenalidomide, or both. In some cases, the subject is classified as having low or intermediate-1 IPSS risk MDS and/or MDS relapsed or refractory to ESA. In certain cases, the subject is non-del5q. In some cases, the subject is classified as IPSS intermediate and poor cytogenetic risk.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1. Study design for Phase 3 trial of imetelstat for treating MDS. *Received at least 8 weeks of ESA treatment (epoetin alfa≥40,000 U, epoetin beta≥30,000 U or darbepoetin alfa 150 mcg or equivalent per week) without Hgb rise ≥1.5 g/dL or decreased RBC transfusion requirement ≥4 units/8 weeks or transfusion dependence or reduction in Hgb by ≥1.5 g/dL after hematologic improvement from ≥8 weeks of ESA treatment. **proportion of patients without any RBC transfusion for at least eight consecutive weeks since entry to the trial (8-week TI); proportion of patients without any RBC transfusion for at least 24 consecutive weeks since entry to the trial (24-week TI). (EPO=Erythropoietin; ESA=Erythropoietin stimulating agents; G-CSF=Granulocyte colony stimulating factor; Hgb=hemoglobin; IPSS=International Prognostic Scoring System; ITT=intent to treat; RBC=red blood cell; HI-E=hematologic improvement-erythroid; HMAs=hypomethylating agents; MDS=myelodysplastic syndromes.)

FIG. 2. Continuous sustained TI with imetelstat treatment. *Pretreatment hemoglobin was 6.2 g/dL with transfusion burden of 5 units/8 weeks before study start; on-study hemoglobin was <6.5 g/dL during majority of TI period, yet no transfusions given. RBC=red blood cell; TI=transfusion independence.

FIG. 3. Highly statistically significant and clinically meaningful durability of TI. *Kaplan-Meier estimates of duration of RBC TI; 8-week TI Responder Analysis Set; Hazard ratio is from the Cox proportional hazard model, stratified by prior RBC transfusion burden (<=6 vs. >6 units RBC) and IPSS risk group (low vs. intermediate-1), with treatment as the only covariate; P-value for superiority of imetelstat versus placebo in hazard ratio is based on stratified log-rank test. CI=confidence interval; HR=hazard ratio; RBC=red blood cell; TI=transfusion independence.

FIG. 4A. Transfusion independence rates assessed over longer periods. Imetelstat increased magnitude of benefit compared to placebo with longer TIs. P-value is based on Cochran Mantel Haenszel test stratified for prior RBC transfusion burden (≤6 units or >6 units of RBCs/8 weeks) and baseline IPSS risk score (Low or Intermediate-1 risk). TI=transfusion independence. FIG. 4B. Rate of ≥1-year TI in imetelstat treated patients versus placebo. FIG. 4C. Rate of ≥1-year TI in imetelstat treated patients showing ≥8-weeks and >24-weeks TI responders.

FIG. 5A. Mean change in hemoglobin levels over time shows highly statistically significant increase in hemoglobin levels after imetelstat treatment compared to placebo. The mean changes from the minimum Hgb of the values that were after 14 days of transfusions in the 8 weeks prior to the first dose date are shown. Data points that have fewer than four patients are not shown. P-value is based on a mixed model for repeated measures with Hgb change as the dependent variable, week, stratification factors, minimum Hgb in the 8 weeks prior to the first dose date, and treatment arm as the independent variables with autoregressive moving average (ARMA(1,1)) covariance structure. Hgb=hemoglobin; SE=standard error; TI=transfusion independence.

FIG. 5B. Mean change in central hemoglobin over time in ≥1 year TI responders. For the patient on placebo: pretreatment hemoglobin was 6.2 g/dL and transfusion burden was 5U/8 weeks; while on study, hemoglobin was <6.5 g/dL during majority of the TI period. BL=baseline.

FIG. 6. Change in RBC transfusion units over time shows statistically significant decrease in number of RBC units transfused after imetelstat treatment compared to placebo. P-value is based on a mixed model for repeated measures with change in RBC transfusion as the dependent variable, week, stratification factors, prior transfusion burden, and treatment arm as the independent variables with autoregressive moving average (ARMA(1,1)) covariance structure. RBC=red blood cell; SE=standard error.

FIGS. 7A-7D. Key subgroup analyses. (A) Eight-week TI by Ring-Sideroblast (RS) status, prior transfusion burden, and IPSS risk category. (B) Twenty-four week TI by RS status, prior transfusion burden, and IPSS risk category. Shown is a forest plot of the primary end point according to subgroups, the percentage difference for imetelstat compared with placebo in the rates of 8-week TI among the subgroups indicated. The 95% CI is based on Wilson Score method. The P value is based on Cochran-Mantel-Haenszel controlling for prior RBC transfusion burden (≥4 to ≤6 vs. >6 units RBC) and International Prognostic Scoring System (IPSS) risk group (low vs. intermediate-1) applied to randomization. Statistically significant 8-week TI rates (p<0.05) and comparable magnitude of clinical benefit demonstrated across all subgroups was observed with imetelstat. *Cochran Mantel Haenszel test stratified for prior RBC transfusion burden (≤6 units or >6 units of RBCs/8 weeks) and baseline IPSS risk score (Low risk or intermediate-1 risk). CI=confidence interval; IPSS=International Prognostic Scoring System; IWG=International Working Group; RBC=red blood cell; RS+=ring sideroblast positive; RS−=ring sideroblast negative. Eight-week TI by (C) baseline TA, TL and hTERT level and (D) baseline TA, TL and hTERT level. (C) and (D) show forest plots of TI responses according to subgroups, the percentage difference for imetelstat compared with placebo in the rates of 8- and 24-week TI among the subgroups indicated. Poor prognosis mutation was defined as presence of TP53, EZH2, ETV6, RUNX1, or ASXL1 based on Bejar et al. N Engl J Med 2011.

FIG. 8. TI for patients with or without RS. Shown are the percentages of patients who had TI for 24 weeks or longer in patients with the indicated status of RS in each study group. For the key secondary end point of TI for ≥24 weeks, there were differences of 28% (P=0.003) in the patients with RS and 21% (P=0.019) in the patients without RS for imetelstat compared with placebo. No patients without RS receiving placebo achieved 24 weeks of TI. Error bars represent the 95% confidence interval calculated by Exact Clopper-Pearson confidence interval. NE denotes not evaluable. Primary (8-week TI) and secondary end point (24-week TI) by RS status shows that both RS+ and RS− demonstrate statistically significant improvement in both 8-week and 24-week TI. *Kaplan-Meier estimates of duration of RBC TI; 8-week/24-week TI Responder Analysis Set; P-value for TI rate is based on Cochran Mantel Haenszel test stratified for prior RBC transfusion burden (≤6 units or >6 units of RBCs/8 weeks) and baseline IPSS risk score (Low or intermediate-1 risk); P-value for duration of TI is based on stratified log-rank test. CI=confidence interval; NE=non evaluable; RS+=ring sideroblast positive; RS−=ring sideroblast negative; TI=transfusion independence.

FIG. 9. Molecular data in imetelstat treated patients shows more than 50% decrease in VAF in SF3B1, TET2, DNMT3A and ASXL1 mutations. Ratios underneath the bars represent the number of patients with ≥50% reduction in VAF as numerator and the total number of patients with detectable assessment (≥5% VAF) in specified mutation at baseline and any post-baseline mutation assessment as denominator. *P-value is based on Cochran Mantel Haenszel test stratified for prior RBC transfusion burden (≤6 units or >6 units of RBCs/8 weeks) and baseline IPSS risk score (Low or Intermediate-1 risk).

FIG. 10. Association between maximum reduction in SF3B1 or TET2 VAF with TI response (at least 8 weeks, 24 weeks, 1 year, and no response) between imetelstat and placebo treatment in patients with baseline VAF ≥5% and at least 1 post-baseline mutation assessment.

FIG. 11. Meaningful TI in patients treated with imetelstat as compared to placebo. The p-value is based on Cochran-Mantel-Haenszel controlling for prior RBC transfusion burden (≤6 vs. ≥6 units RBC) and IPSS risk group (low vs. intermediate-1) applied to randomization.

FIGS. 12A-12C. Reduction in VAF of SF3B1, TET2, DNMT3A, and ASXL1 mutations and correlations with longer duration of TI and increased hemoglobin (A) shows the comparison between each study group in maximum percentage change during treatment period from baseline in mutant VAF of the indicated gene. In 3 of 4 genes frequently mutated in MDS, VAF reduction was significantly greater in patients treated with imetelstat than those treated with placebo: SF3B1 (P<0.001), TET2 (P=0.032), DNMT3A (P=0.019) and ASXL1 (P=NS). (B) shows the correlation between maximum percentage reduction from baseline by imetelstat treatment in SF3B1 VAF and duration of transfusion independence in the imetelstat group. The result demonstrates the greater reduction in SF3B1 VAF, the longer TI duration (n=78, Pearson's r=−0.549, P<0.001). (C) shows the correlation between the maximum percentage reduction from baseline in SF3B1 VAF and maximum change from pretreatment value in hemoglobin levels in the imetelstat group. The result demonstrates the greater reduction in SF3B1 VAF, the higher increase in hemoglobin (n=67, Pearson's r=−0.626, P<0.001). Hemoglobin measures from blood drawn within 14 days after a transfusion were excluded. Mutation analyses included patients with baseline detectable variant allele ≥5% and at least one postbaseline mutation assessment.

FIGS. 13A-13B. Time to first sustained meaningful improvement in fatigue and the association of TI and HI-E response with sustained meaningful improvement in fatigue. Sustained improved in fatigue in imetelstat treated patients. A. Kaplan Meier survival estimates of time to first sustained improvement in FACIT Fatigue Scale, PRO population. B. A graph showing percentage of subjects having sustained meaningful improvement in fatigue for subjects having ≥8 week or ≥24 week Transfusion Independence (TI) and HI-E per IWG 2006 criteria responses by imetelstat responders, imetelstat non-responders, placebo responders and placebo non-responders. Sustained, meaningful improvement in fatigue was defined as an increase of at least 3 points on the FACIT Fatigue for at least 2 consecutive nonmissing cycles. Analyses included patients with available fatigue data at baseline.

FIG. 14. IMerge Phase 3: Study design and patient disposition.

FIG. 15. Swimmer plot of TI intervals in patients with ≥8-week TI.

FIG. 16. Relative dose intensity (%) per cycle. Shown is the relative dose intensity with respect to the starting dose 7.5 mg/kg.

FIGS. 17A-17D. Reduction in VAF of TET2 and DNMT3A mutations and correlations with longer duration of TI and increased hemoglobin. (A) shows the correlation between maximum percentage reduction from baseline by imetelstat treatment in TET2 VAF and duration of transfusion independence in the imetelstat group. The result demonstrates the greater reduction in TET2 VAF, the longer TI duration (n=38, Pearson's r=−0.577, P<0.001). (B) shows the correlation between maximum percentage reduction from baseline by imetelstat treatment in DNMT3A VAF and duration of transfusion independence in the imetelstat group. Fitted Duration of RBC TI on Maximum Percentage Reduction from Baseline in DNMT3A VAF in Imetelstat Arm. The fitted line and p-value are based on linear regression with duration of RBC TI as dependent variable, and the maximum percentage reduction from baseline in DNMT3A VAF as independent variable. The correlation coefficient is based on Pearson Correlation. N represents imetelstat arm subjects in mutation biomarker analysis set who had detectable assessment (>=5) in DNMT3A at baseline and had any post-baseline mutation assessment. The result demonstrates the greater reduction in DNMT3A VAF, the longer TI duration (n=18, Pearson's r=−0.545, P<0.019). (C) shows the correlation between the maximum percentage reduction from baseline in TET2 VAF and maximum change from pretreatment value in hemoglobin levels in the imetelstat group. The result demonstrates the greater reduction in TET2 VAF, the higher increase in hemoglobin (n=32, Pearson's r=−0.659, P<0.001). Hemoglobin measures from blood drawn within 14 days after a transfusion were excluded. Mutation analyses included patients with baseline detectable variant allele ≥5% and at least one postbaseline mutation assessment. (D) shows the correlation between the maximum percentage reduction from baseline in DNMT3A VAF and maximum change from pretreatment value in hemoglobin levels in the imetelstat group. The result demonstrates the greater reduction in DNMT3A VAF, the higher increase in hemoglobin (n=13, Pearson's r=−0.667, P<0.013). Hemoglobin measures from blood drawn within 14 days after a transfusion were excluded. Mutation analyses included patients with baseline detectable variant allele ≥5% and at least one postbaseline mutation assessment.

FIG. 18. Imetelstat treatment resulted in sustained reduction of SF3B1 VAF over time.

FIGS. 19A-19B. 8-week (19A) and 24-week (19B) RBC-TI correlated with Reduction in RS+ cells, cytogenetic responses, and VAF reduction in patients treated with imetelstat. P values calculated using Fisher exact test between yes vs. no in each outcome. BM, bone marrow; CR, complete response; IRC, independent review committee; PR, partial response.

FIG. 20. A higher percentage of patients treated with imetelstat vs placebo had a ≥50% reduction in central bone marrow RS. Also, RBC-TI responders were higher in patients achieving ≥50% reduction in central bone marrow RS.

FIG. 21. Swimmer plot of 22 LR-MDS patients (21, imetelstat treated and 1 placebo treated patient) who achieved RBC TI of ≥1 year. Patient taking placebo: pretreatment Hb level was 6.2 g/dL and transfusion burden of 5 U per 8 weeks before study start, whereas on-study, Hb level was <6.2 g/dL during most of the TI period, with no transfusions given. PBO: placebo.

FIG. 22. Summary of TI-response rates by baseline mutation status of the most frequently mutated genes.

FIG. 23. Heatmap of changes in mutational burden in 18 imetelstat treated patients with ≥1 year TI.

FIG. 24 shows TI rates by number of baseline mutations.

FIG. 25. Summary of TI response rates by baseline mutation status of 4 sets of genes involved in different biological functions.

FIGS. 26A-26C. Spaghetti plots of mutation VAF change over time in imetelstat treated patients. Reduction in VAF over time with different patterns for different genes upon imetelstat treatment are shown. Dose of 4.7 mg/kg represents the actual dose from 4.2 to 5.35 mg/kg. Dose of 6.0 mg/kg represents the actual dose of 5.35 to 6.75 mg/kg. Dose of 7.5 mg/kg represents the actual dose of 6.75001 to 8.45 mg/kg. Data from scheduled and unscheduled visits are included.

FIGS. 27A-27D. Limited reduction of VAF of certain genes over time in placebo treated patients. Dose of 6.0 mg/kg represents the actual dose of 5.35 to 6.75 mg/kg. Dose 7.5 mg/kg represents the actual dose of 6.75001 to 8.45 mg/kg. Dose of 9.4 mg/kg represents the actual dose of 8.45 to 11 mg/kg. Data from scheduled and unscheduled visits are included.

### DETAILED DESCRIPTION

This application provides methods of treating and methods of monitoring therapeutic efficacy in a subject with MDS. Also provided are methods of identifying a subject with MDS suitable for treatment with a telomerase inhibitor. The subject methods can include administering to the subject an effective amount of a telomerase inhibitor and assessing the VAF for one or more of the following genes: SF3B1, TET2, DNMT3A and ASXL1 in a biological sample obtained from the subject. In some cases, 25% or more reduction in VAF for one or more of the following genes: SF3B1, TET2, DNMT3A and ASXL1 identifies a subject who has an increased likelihood of benefiting from treatment with the telomerase inhibitor. The subject methods can include administering to the subject an effective amount of a telomerase inhibitor and assessing the VAF for one or more of the following genes: SF3B1, TET2, DNMT3A, CUX1, and ASXL1 in a biological sample obtained from the subject. In some cases, 25% or more reduction in VAF for one or more of the following genes: SF3B1, TET2, DNMT3A, CUX1 and ASXL1 identifies a subject who has an increased likelihood of benefiting from treatment with the telomerase inhibitor.

In some cases, treatment with a telomerase inhibitor, such as imetelstat is continued if a 25% or more reduction in VAF for one or more of the following genes: SF3B1, TET2, DNMT3A and ASXL1 is observed in a subject. Thus, in some cases, methods of treating MDS in a subject with a telomerase inhibitor, such as imetelstat, comprise administering to the subject a telomerase inhibitor, such as imetelstat and continuing administering to the subject a telomerase inhibitor, such as imetelstat because, after administration of the telomerase inhibitor, such as imetelstat, 25% or more reduction in the VAF was observed for one or more of the following genes: SF3B1, TET2, DNMT3A and ASXL1. The reduction in VAF may be 10% or more, or 15% or more, or 20% or more, or 25% or more, such as 30% or more, 35% or more, 40% or more, 45% or more, 50% or more, 55% or more, 60% or more, 65% or more, 70% or more, 75% or more, 80% or more, 85% or more, 90% or more, or 95% or more, or 100% reduction in the VAF for one or more of the following genes: SF3B1, TET2, DNMT3A and ASXL1.

In some cases, treatment with a telomerase inhibitor, such as imetelstat is continued if a 25% or more reduction in VAF for one or more of the following genes: SF3B1, TET2, DNMT3A, CUX1 and ASXL1 is observed in a subject. Thus, in some cases, methods of treating MDS in a subject with a telomerase inhibitor, such as imetelstat, comprise administering to the subject a telomerase inhibitor, such as imetelstat and continuing administering to the subject a telomerase inhibitor, such as imetelstat because, after administration of the telomerase inhibitor, such as imetelstat, 25% or more reduction in the VAF was observed for one or more of the following genes: SF3B1, TET2, DNMT3A, CUX1, and ASXL1. The reduction in VAF may be 10% or more, or 15% or more, or 20% or more, or 25% or more, such as 30% or more, 35% or more, 40% or more, 45% or more, 50% or more, 55% or more, 60% or more, 65% or more, 70% or more, 75% or more, 80% or more, 85% or more, 90% or more, or 95% or more, or 100% reduction in the VAF for one or more of the following genes: SF3B1, TET2, DNMT3A, ASXL1, and CUX1

A treatment with a telomerase inhibitor may be discontinued if 25% or more reduction is not observed in the VAF for one or more of the following genes: SF3B1, TET2, DNMT3A and ASXL1 in a subject after administration of a telomerase inhibitor and additional factors suggest discontinuing the treatment. A treatment with a telomerase inhibitor may be discontinued if 25% or more reduction is not observed in the VAF for one or more of the following genes: SF3B1, TET2, DNMT3A, CUX1 and ASXL1 in a subject after administration of a telomerase inhibitor and additional factors suggest discontinuing the treatment. For example, if no TI is achieved and/or adverse side-effects are observed, a treatment with a telomerase inhibitor may be discontinued.

The subject can be naive to treatment with a HMA, lenalidomide, or both. In some cases, the subject treated is classified as having: low IPSS risk MDS, intermediate-1 IPSS risk MDS, MDS relapsed to ESA, MDS refractory to MS, or combination thereof. The subject may also be non-del5q. For clarity of disclosure, and not by way of limitation, the detailed description of the invention is divided into subsections that describe or illustrate certain features, embodiments, or applications of the present invention. In some embodiments, the subject is diagnosed as having trisomy 8.

### A. Definitions

SF3B1 (splicing factor 3b subunit 1) is the largest subunit of the SF3B complex and functions by serving as a core component of the U2 snRNP, which is critical for branch site recognition and for the early stages of spliceosome assembly. SF3B1 hot-spot mutations may be selected from E622D, R625C/L/G, H662Q/N/D/Y, T663P, K666R/T/Q/N, K700E, A744P, and/or E783K.

TET2 (Tet Methylcytosine Dioxygenase 2), The protein encoded by this gene is a methylcytosine dioxygenase that catalyzes the conversion of methylcytosine to 5-hydroxymethylcytosine.

ASXL1 (additional sex combs like 1) gene codes for a polycomb chromatin-binding protein and is involved in epigenetic regulation of gene expression.

DNMT3A (DNA methyltransferase 3 alpha) gene codes for an enzyme that catalyzes the transfer of methyl groups to specific CpG structures in DNA, a process called DNA methylation.

CUX1 (cut like homeobox 1) gene codes for a tumor suppressor.

As used herein, the term “about” when referring to a measurable value such as an amount, a temporal duration, and the like, is meant to encompass variations of between ±20% and ±0.1%, preferably ±20% or ±10%, more preferably ±5%, even more preferably ±1%, and still more preferably ±0.1% from the specified value, as such variations are appropriate to perform the disclosed methods.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, and the like; and when the molecule contains a basic functionality, salts of organic or inorganic acids, such as hydrochloride, and the like. Pharmaceutically acceptable salts of interest include, but are not limited to, aluminum, ammonium, arginine, barium, benzathine, calcium, cholinate, ethylenediamine, lysine, lithium, magnesium, meglumine, procaine, potassium, sodium, tromethamine, N-methylglucamine, N,N′-dibenzylethylene-diamine, chloroprocaine, diethanolamine, ethanolamine, piperazine, zinc, diisopropylamine, diisopropylethylamine, triethylamine and triethanolamine salts.

The term “salt(s) thereof” means a compound formed when a proton of an acid is replaced by a cation, such as a metal cation or an organic cation and the like. Preferably, the salt is a pharmaceutically acceptable salt. By way of example, salts of the present compounds include those wherein the compound is protonated by an inorganic or organic acid to form a cation, with the conjugate base of the inorganic or organic acid as the anionic component of the salt. Salts of interest include, but are not limited to, aluminum, ammonium, arginine, barium, benzathine, calcium, cesium, cholinate, ethylenediamine, lithium, magnesium, meglumine, procaine, N-methylglucamine, piperazine, potassium, sodium, tromethamine, zinc, N,N′-dibenzylethylene-diamine, chloroprocaine, diethanolamine, ethanolamine, piperazine, diisopropylamine, diisopropylethylamine, triethylamine and triethanolamine salts. It is understood that for any of the oligonucleotide structures depicted herein that include a backbone of internucleoside linkages, such oligonucleotides may also include any convenient salt forms. In some embodiments, acidic forms of the internucleoside linkages are depicted for simplicity. In some instances, the salt of the subject compound is a monovalent cation salt. In certain instances, the salt of the subject compound is a divalent cation salt. In some instances, the salt of the subject compound is a trivalent cation salt. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include for example cis-trans isomers, E and Z isomers, enantiomers, and diastereomers. As to any of the groups disclosed herein which contain one or more substituents, it is understood, of course, that such groups do not contain any substitution or substitution patterns which are sterically impractical and/or synthetically non-feasible. All stereoisomers are intended to be included within the scope of the present disclosure.

A person of ordinary skill in the art would recognize that other tautomeric arrangements of the groups described herein are possible. It is understood that all tautomeric forms of a subject compound are encompassed by a structure where one possible tautomeric arrangement of the groups of the compound is described, even if not specifically indicated.

It is intended to include a solvate of a pharmaceutically acceptable salt of a tautomer of a stereoisomer of a subject compound. These are intended to be included within the scope of the present disclosure.

Before certain embodiments are described in more detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used throughout, “MDS” refers to myelodysplastic syndrome or myelodysplastic syndromes.

The term “one or more of the following genes: SF3B1, TET2, DNMT3A and ASXL1” indicates these genes in singularity or in any combination 2, 3 or 4 of these genes. A skilled artisan can readily identify that 6 combinations of any two of these genes can be made, 4 combinations of any three of these genes can be made, and one combination with all of these genes can be made. The term “one or more of the following genes: SF3B1, TET2, DNMT3A, CUX1, and ASXL1” indicates these genes in singularity or in any combination of 2, 3, 4, or 5 of these genes. A skilled artisan can readily identify that 10 combinations of any two of these genes can be made, 10 combinations of any three of these genes can be made, 5 combinations of any four of these genes can be made, and one combination with all of these genes can be made.

Moreover, one or more genes can be selected from a subset of the following genes: SF3B1, TET2, DNMT3A and ASXL1. One or more genes can be selected from a subset of the following genes: SF3B1, TET2, DNMT3A, CUX1 and ASXL1. For example, one or more genes can be selected from the following genes: TET2, DNMT3A and ASXL1. One or more or more genes can also be selected from the group of the following genes: SF3B1, TET2, and DNMT3A. Alternatively, one or more genes can be selected from the following genes: SF3B1, DNMT3A and ASXL1. Even further, one or more genes can be selected from the following genes: SF3B1, TET2, and ASXL1. Further one or more genes can be selected from SF3B1, TET2, CUX1 or from SF3B1, DNMT3A, CUX1 or from SF3B1, TET2, CUX1 or from TET2, DNMT3A, CUX1. Further, one or more genes selected from any one of the following groups of genes: SF3B1 and TET2; SF3B1 and DNMT3A; SF3B1 and ASXL1; TET2 and DNMT3A; TET2 and ASXL1; DNMT3A and ASXL1; SF3B1 and CUX1; TET2 and CUX1; DNMT3A and CUX1; ASXL1 and CUX1. In some cases, VAF for one gene is analyzed from the following genes: SF3B1, TET2, DNMT3A, ASXL1, and CUX1.

A biological sample obtained from the subject may be a peripheral blood sample or bone marrow sample, for example, cells from peripheral blood or bone marrow obtained from the subject.

Using each of these combinations in the methods as disclosed herein is within the purview of the invention. Also, the comparison of VAF between two time-points is carried out for the same combination of genes.

### B. Pharmacodynamics (PD)

The present disclosure is based in part on a pharmacodynamic effect demonstrating an association between response to telomerase inhibition therapy, particularly, imetelstat therapy, in subjects with a MDS and 25% or more reduction in VAF for one or more of the following genes: SF3B1, TET2, DNMT3A and ASXL1. In some cases, the subjects that exhibit 25% or more reduction in VAF for one or more of the following genes: SF3B1, TET2, DNMT3A and ASXL1 typically show at least 8-week TI response. Conversely, the subject that do not show such 25% or more reduction typically do not show at least 8-week TI response.

Thus, the present disclosure provides for stratification and identification or selection of patients likely to benefit from telomerase inhibition therapy for MDS, and provides methods of monitoring response, relapse, and prognosis in subjects undergoing such treatment.

Aspects of the present disclosure include methods of identifying or selecting subjects with MDS for treatment with a telomerase inhibitor, such as imetelstat, and methods of treating MDS. Methods of monitoring therapeutic efficacy in a subject with MDS are also provided. In some cases, the pharmacodynamic effect on which an embodiment of the subject methods is based is 25% or more reduction in VAF for one or more of the following genes: SF3B1, TET2, DNMT3A and ASXL1.

In some cases, the 25% or more reduction in VAF for one or more of the following genes: SF3B1, TET2, DNMT3A, ASXL1, and CUX1 is exhibited after at least 4 weeks, at least 6 weeks, at least 8 weeks, after at least 10 weeks, or at least 12 weeks from initial administration of a telomerase inhibitor, such as imetelstat administration. Such VAF determination can be performed on cells from peripheral blood or bone marrow obtained from a patient. A peripheral blood or bone marrow sample can be periodically obtained from a patient, for example, every 4-6 weeks. In some cases, a peripheral blood or bone marrow sample is obtained from a patient whenever a telomerase inhibitor is administered to the patient. Typically, for monitoring a telomerase inhibitor therapy or determining continuation or discontinuation of a telomerase inhibitor therapy, VAF for the target genes can be measured before, particularly, immediate before, for example, less than about 4-6 weeks before administering the first dose of the telomerase inhibitor, e.g., imetelstat. The VAF for the target genes can be measured after a telomerase inhibitor administration at an interval of 4-16 weeks, e.g., at an interval of 4 weeks, 6 weeks, 8 weeks, 10 weeks, 11 weeks, 12 weeks, 13 weeks, 14 weeks, or 16 weeks. For continuous monitoring of the therapy, VAF can be determined repeatedly after each such interval, for example, after every 4 weeks, every 6 weeks, every 8 weeks, every 10 weeks, every 11 weeks, every 12 weeks, every 13 weeks, every 14 weeks, or every 16 weeks.

VAF for the target genes measured before, particularly, immediate before, for example, less than about 4-6 weeks before administering the first dose of the telomerase inhibitor, e.g., imetelstat, is referenced as “baseline VAF.”

VAF for the target genes can be determined and/or measured using any convenient methods. In one such method, whole blood collected in an EDTA tube is used to perform Next Generation Sequencing (NGS), for example, using LeukoVantage®. NGS using any other sequencing platform can be used, for example, nanopore sequencing, paired-end sequencing, Ion Torrent sequencing, and single molecule real time (SMRT) sequencing. An MDS gene panel can be used. For example, a gene panel containing one or more of SF3B1, TET2, DNMT3A and ASXL1 can be used. In a specific embodiment, a gene panel that includes the following 36 genes was used: ASXL1, ATM, BCOR, CBL, CEBPA, CSF3R, CUX1, DNMT3A, ETNK1, ETV6, EZH2, FLT3, GATA2, IDH1, IDH2, IKZF1, JAK2, KRAS, NF1, NPM1, NRAS, PHF6, PTEN, PTPN11, RUNX1, SETBP1, SF3B1, SRSF2, STAG2, STAT3, STK11, TET2, TP53, U2AF1, WT1, ZRSR2. The NGS assay can be performed using DNA bait capture methodology on the NextSeq® (Illumina®) platform. Following DNA extraction from leukocytes, a targeted, amplicon-based NGS method is used to detect mutations. The lower limit of sensitivity is 5% which is superior to that of traditional Sanger DNA sequencing. The percentage of mutation reads will be reported and can be used to assess the size of the clonal population. VAF is the percentage of sequence reads observed matching a specific DNA variant divided by the overall coverage at that locus. Because NGS provides a near random sample, VAF is thus a surrogate measure of the proportion of DNA molecules in the original specimen carrying the variant.

As noted above, a decrease in VAF implies a decrease in malignant cell burden. For example, 10% or more, or 15% or more, or 20% or more, or 25% or more, such as 30% or more, 35% or more, 40% or more, 45% or more, 50% or more, 55% or more, 60% or more, 65% or more, 70% or more, 75% or more, 80% or more, 85% or more, 90% or more, or 95% or more, or 100% reduction in the VAF for one or more of the following genes: SF3B1, TET2, DNMT3A, ASXL1, and CUX1 indicates that a subject is has an increased likelihood of benefiting from treatment with the telomerase inhibitor.

In some cases, a decrease in VAF implies a longer duration for TI for the subject. For example, a decrease in VAF may be correlated to a longer duration for TI for the subject as compared to a subject that does not have a similar decrease in VAF. In some cases, a decrease in VAF implies a shorter time to TI onset in the subject. For example, a decrease in VAF may be correlated to a shorter time to TI onset for the subject as compared to a subject that does not have a similar decrease in VAF.

### C. Treatment

In some cases, the present disclosure describes methods of treating a MDS with a telomerase inhibitor in a subject that is naive to treatment with particular agents, e.g., an agent selected from a hypomethylating agent (HMA) and lenalidomide. A subject is considered to be treatment “naive” if the subject has never undergone a particular treatment for an illness. Treatment of patients with MDS relapsed or refractory to an ESA therapy with imetelstat can improve outcomes, including lower incidence of anemia.

A subject is a mammal in need of treatment for MDS. Generally, the subject is a human patient. In some embodiments of the invention, the subject can be a non-human mammal such as a non-human primate, an animal model (e.g., animals such as mice and rats used in screening, characterization, and evaluation of medicaments) and other mammals. As used herein, the terms patient and subject are used interchangeably.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving a treatment.

In certain instances, the subject method provides an enhanced therapeutic response in those subjects who have not previously been treated with a hypomethylating agent (HMA) or lenalidomide, relative to subjects who were so treated previously. By “enhanced therapeutic response” is meant a statistically significant improvement in a primary and/or secondary end point of MDS therapy and/or amelioration of one or more symptoms of MDS (e.g., as described herein), e.g., rate and/or duration of red blood cell RBC TI, or hematologic improvement (HI) rate relative to an appropriate control. In some cases, the subject methods provide a therapeutic effect of RBC TI, e.g., lasting 4 weeks or longer, such as 5 weeks or longer, 6 weeks or longer, 7 weeks or longer, 8 weeks or longer, 9 weeks or longer, 10 weeks or longer, 12 weeks or longer, 16 weeks or longer, 20 weeks or longer, 24 weeks or even longer. In some instances, time to TI and/or duration of TI is significantly improved. In certain instances, the subject method provides a duration of TI that is 24 weeks or longer, such as 30 weeks or longer, 36 weeks or longer, 42 weeks or longer, 48 weeks or longer, 60 weeks or longer, or even longer.

A hypomethylating agent (HMA) is an agent that inhibits DNA methylation, e.g., by blocking the activity of DNA methyltransferase (DNA methyltransferase inhibitors/DNMT inhibitors). HMAs of interest include, but are not limited to, decitabine (CAS Registry Number: 2353-33-5; 5-aza-2′-deoxycytidine), azacitidine (CAS Registry Number: 320-67-2,5-azacytidine) and guadecitabine (SGI-110). In some instances, the subject is treatment naive to decitabine. In some instances, the subject is treatment naive to azacitidine. In other instances, the subject is treatment naive to both decitabine and azacitidine.

Lenalidomide is a drug that is used to treat a variety of inflammatory disorders and cancers, including multiple myeloma and MDS. Lenalidomide (CAS Registry Number: 191732-72-6; 2,6-Piperidinedione, 3-(4-amino-1,3-dihydro-1-oxo-2H-isoindol-2-yl)-); 3-(4-Amino-1-oxoisoindolin-2-yl)piperidine-2,6-dione) is a derivative of thalidomide. Lenalidomide has various mechanisms of action that provide a broad range of biological activities that can be exploited to treat a variety of hematologic and solid cancers. In some instances, the subject is treatment naive to lenalidomide.

Deletion 5q (del5q) refers to a chromosomal abnormality found in particular forms of MDS subjects (Adema et al., Haematologica. 2013 December; 98(12): 1819-1821; Sole et al., Haematologica. 2005; 90(9): 1168-78). In some cases of the subject methods, the subject is a human patient who has del5q. In some cases, the subject is a human patient who is non-del5q. A non-del5q subject is a subject that does not have the del5q chromosomal abnormality. In certain cases, the non-del5q subject is human.

In certain instances, the subject has not received prior treatment with either a hypomethylating agent (HMA) or lenalidomide and does not have a del(5q) chromosomal abnormality (e.g., is non-del5q). In certain cases, the non-del5q subject is human.

In certain embodiments, the subject is a human patient with intermediate or poor cytogenetic risk. In some instances, the subject is a human patient diagnosed as having trisomy 8. In certain instances, the subject is a human patient with trisomy 8 mosaicism. In other instances, the subject is a human patient with trisomy 8 without mosaicism. The term “mosaicism” is used herein in its conventional sense to refer to a condition in which cells within the subject have different genetic makeup. In human patients diagnosed as having trisomy 8 with mosaicism, some of the subject's cells have three copies of chromosome 8 while other cells have two copies of chromosome 8.

In certain embodiments, methods include identifying a subject with MDS for treatment with a telomerase inhibitor, such as imetelstat, where the method includes: identifying a subject as having trisomy 8 (with or without mosaicism); measuring VAF in one or more of: SF3B1, TET2, DNMT3A, ASXL1, and CUX1 genes in a biological sample obtained from the patient after administration of a telomerase inhibitor; and comparing the VAF in one or more of: SF3B1, TET2, DNMT3A, ASXL1, and CUX1 genes in the biological sample to a baseline VAF in one or more of: SF3B1, TET2, DNMT3A and ASXL1 genes prior to administration of the telomerase inhibitor; wherein a 25% or more reduction in the VAF for one or more of: SF3B1, TET2, DNMT3A, ASXL1, and CUX1 genes identifies a patient who has an increased likelihood of benefiting from treatment with the telomerase inhibitor.

In other embodiments, methods include treating MDS in a subject diagnosed as having trisomy 8, where the method includes: identifying a subject diagnosed as having trisomy 8; administering to the subject an effective amount of a telomerase inhibitor; and assessing VAF in one or more of: SF3B1, TET2, DNMT3A, ASXL1, and CUX1 genes in a biological sample obtained from the patient after administration of a telomerase inhibitor, such as imetelstat. The methods then comprise continuing administering the telomerase inhibitor to the subject if 25% or more reduction in the VAF for one or more of: SF3B1, TET2, DNMT3A, ASXL1, and CUX1 genes is observed.

In yet other embodiments, methods include monitoring therapeutic efficacy in a subject with MDS, where the method includes: measuring VAF in one or more of: SF3B1, TET2, DNMT3A, ASXL1, and CUX1 genes in a biological sample obtained from a patient diagnosed as having trisomy 8 after administration of a telomerase inhibitor; and comparing the VAF in one or more of: SF3B1, TET2, DNMT3A, ASXL1, and CUX1 genes in the biological sample to a baseline VAF in one or more of: SF3B1, TET2, DNMT3A, ASXL1, and CUX1 genes prior to administration of the telomerase inhibitor; wherein a 25% or more reduction in the VAF for one or more of: SF3B1, TET2, DNMT3A, ASXL1, and CUX1 genes identifies a patient who has an increased likelihood of benefiting from treatment with the telomerase inhibitor.

In methods according to certain embodiments, the subject is a patient diagnosed as having trisomy 8. In other embodiments, the subject is a patient diagnosed as having trisomy 8 and is naïve to treatment with an agent selected from an HMA, lenalidomide, and combination thereof. In other embodiments, the subject is a patient diagnosed as having trisomy 8 and is a non-del5q human patient. In still other embodiments, the subject is a patient diagnosed as having trisomy 8 and the MDS is relapsed or refractory MDS, such as MDS relapsed or refractory to ESA.

In some cases, the disclosure provides a use of a telomerase inhibitor, such as imetelstat for treating and/or monitoring therapeutic efficacy in a subject with MDS. Also provided is a method of identifying a subject with MDS suitable for treatment with a telomerase inhibitor. The use comprises assessing the VAF for one or more of the following genes: SF3B1, TET2, DNMT3A, ASXL1, and CUX1 in a biological sample obtained from the subject after administration of a telomerase subject to the subject. In some cases, 25% or more reduction in VAF for one or more of the following genes: SF3B1, TET2, DNMT3A, ASXL1, and CUX1 identifies a subject who has an increased likelihood of benefiting from treatment with the telomerase inhibitor.

In some cases, use of a telomerase inhibitor for treating MDS in a subject is continued if a 25% or more reduction in VAF for one or more of the following genes: SF3B1, TET2, DNMT3A, ASXL1, and CUX1 is observed in a subject. A treatment with a telomerase inhibitor may be discontinued if 25% or more reduction is not observed in the VAF for one or more of the following genes: SF3B1, TET2, DNMT3A, ASXL1, and CUX1 in a subject after administration of a telomerase inhibitor and additional factors suggest discontinuing the treatment. For example, if no TI is achieved and/or adverse side-effects are observed, a treatment with a telomerase inhibitor may be discontinued. For example, a treatment with a telomerase inhibitor may be discontinued if 75% or less, or 70% or less, or 60% or less, or 55% or less, or 50% or less, or 45% or less, or 40% or less, or 35% or less, or 30% or less, or 25% or less, or 20% or less, or 15% or less, or 10% or less, or 5% or less reduction in the VAF for one or more of the following genes: SF3B1, TET2, DNMT3A, ASXL1, and CUX1 is measured from the biological sample from the subject. In such cases, a low reduction in the VAF as described above may indicate that a subject is has a decreased likelihood of benefiting from treatment with the telomerase inhibitor.

The subject can be naive to treatment with a HMA, lenalidomide, or both. In some cases, the subject is classified as having: low IPSS risk MDS, intermediate-1 IPSS risk MDS, MDS relapsed to ESA, MDS refractory to MS, or combination thereof. The subject may also be non-del5q. For clarity of disclosure, and not by way of limitation, the detailed description of the invention is divided into subsections that describe or illustrate certain features, embodiments, or applications of the present invention. In some embodiments, the subject is diagnosed as having trisomy 8.

### D. Myelodysplastic syndrome (MDS)

MDS is a group of diseases that includes cancers of the blood and bone marrow, which in some cases can be characterized by cytopenias resulting from ineffective haemopoiesis. A variety of MDS can be treated using the subject methods, including but are not limited to, diseases such as refractory anemia, refractory anemia with excess blasts, refractory cytopenia with multilineage dysplasia, refractory cytopenia with unilineage dysplasia, chronic myelomonocytic leukemia, MDS with isolated del (5q) and MDS unclassifiable.

MDS is characterized by clonal myeloproliferation arising from malignant progenitor cell clones that have shorter telomeres and multiple clonal genetic abnormalities. Telomerase activity (TA) and expression of human telomerase reverse transcriptase (hTERT) is significantly increased in MDS and may play a role in dysregulated cell growth, leading to continued and uncontrolled proliferation of malignant progenitor cell clones. Higher TA and hTERT as well as shorter telomere length are poor prognostic features for patients with low-risk MDS, leading to shorter overall survival. There are limited treatment options for anemia in lower-risk MDS that has relapsed after or is refractory to ESA therapy. Targeting MDS clones with imetelstat can improve outcomes, including anemia, in patients with MDS relapsed or refractory to an ESA therapy.

In some embodiments, the subject methods find use in alleviating at least one symptom associated with myelodysplastic syndrome, such as, e.g., refractory anemia, refractory anemia with excess blasts, refractory cytopenia with multilineage dysplasia, refractory cytopenia with unilineage dysplasia, and chronic myelomonocytic leukemia. In some embodiments, the symptoms include shortness of breath, fatigue, weakness, fainting, nosebleeds, bruising, bleeding from mouth or gums, bloody stool, petechiae, or stroke.

In some instances, the subject has a relapsed or refractory MDS. “Refractory MDS” refers to patients who still have MDS cells in their bone marrow after treatment with any convenient MDS-related therapy. “Relapsed MDS” refers to patients who have a return of MDS cells in their bone marrow and a decrease in normal blood cells after remission. In certain instances, the subject has MDS relapsed or refractory to ESA. ESAs can increase hemoglobin levels and abolish transfusion dependence for a period in some MDS cases. ESAs of interest include but are not limited to erythropoietin-alpha, erythropoietin-beta, and darbepoetin.

In some embodiments, the subject is identified as a human patient having trisomy 8. In certain instances, the subject is a human patient with trisomy 8 mosaicism. In other instances, the subject is a human patient with trisomy 8 without mosaicism.

In certain embodiments of the subject method, the subject is classified as a low or intermediate-1 IPSS risk MDS subject. Myelodysplastic syndromes (MDS) patients can be divided into lower-risk groups (low and intermediate-1 [INT-1] IPSS), in which apoptotic events in the marrow are prevalent and there is a defective response to cytokines (including erythropoietin), and higher-risk groups (intermediate-2 [INT-2] and high IPSS), in which a block in the maturation of marrow progenitors is the principal alteration. In some cases, transfusion dependence is a negative prognostic variable. As such, in certain embodiments of the method, the subject is RBC transfusion dependent. In some cases, the transfusion-dependent subject has an RBC transfusion requirement of about 4 units or more over 8 weeks; or from 4-14 units over an 8-week period, or about 6 units or more per 8 weeks prior to administration according to the subject method. A unit of packed red blood cells (PRBCs) can be about 300 mL/unit. A unit of Whole blood can be about 450-500 rnL/unit.

The International Prognostic Scoring System (IPSS) is a system developed for staging MDS. The IPSS rates 3 factors: the percentage of leukemic blast cells in the bone marrow cells (scored on a scale from zero to 2); chromosome abnormalities, if any, in the marrow cells (scored from zero to 1); and the presence of one or more low blood cell counts (scored as zero or 0.5). Each factor is given a score, with the lowest scores having the best outlook. Then the scores for the factors are added together to make the IPSS score. The IPSS puts people with MDS into 4 groups: low risk; intermediate-1 risk; intermediate-2 risk; and high risk.

### E. Telomerase Inhibitors

Any convenient telomerase inhibitors can find use in the subject methods. In some embodiments, the telomerase inhibitor is an oligonucleotide with telomerase inhibiting activity, in particular an oligonucleotide as defined in WO 2005/023994 and/or WO 2014/088785, the disclosures of which are herein incorporated by reference in their entirety. In some cases, one or more than one telomerase inhibitor (e.g., two or three telomerase inhibitors) can be administered to a mammal to treat a hematological malignancy.

### Imetelstat

In certain embodiments, the telomerase inhibitor is imetelstat, including tautomers thereof and salts thereof, e.g., pharmaceutically acceptable salts. Imetelstat is a novel, first-in-class telomerase inhibitor with clinical activity in hematologic malignancies (Baerlocher et al., NEJM 2015; 373:920-928; Tefferi et al., NEJM 2015; 373:908-919) (shown below):

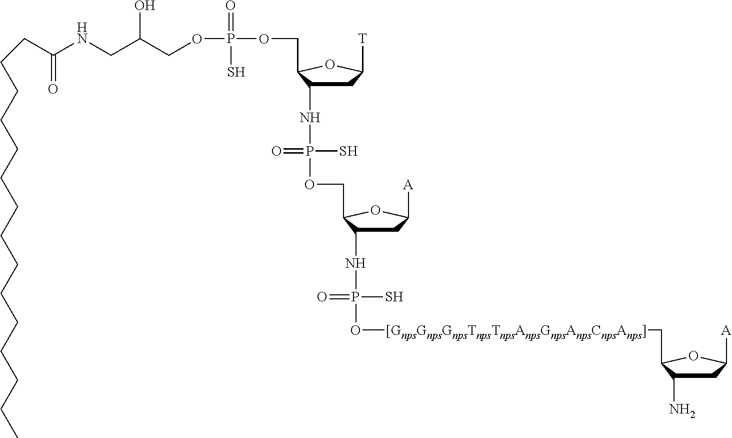

where “nps” represents a thiophosphoramidate linkage —NH—P(═O)(SH)—O—, connecting the 3′-carbon of one nucleoside to the 5′-carbon of the adjacent nucleoside.

In certain instances, the telomerase inhibitor is imetelstat sodium including tautomers thereof. Imetelstat sodium is the sodium salt of imetelstat, which is a synthetic lipid-conjugated, 13-mer oligonucleotide N3′→P5′-thiophosphoramidate. Imetelstat sodium is a telomerase inhibitor that is a covalently-lipidated 13-mer oligonucleotide (shown below) complimentary to the human telomerase RNA (hTR) template region. The chemical name for imetelstat sodium is: DNA, d(3′-amino-3′-deoxy-P-thio) (T-A-G-G-G-T-T-A-G-A-C-A-A), 5′-[O-[2-hydroxy-3-(hexadecanoylamino)propyl]phosphorothioate], sodium salt (1:13) (SEQ ID NO: 1). Imetelstat sodium does not function through an anti-sense mechanism and therefore lacks the side effects commonly observed with such therapies.

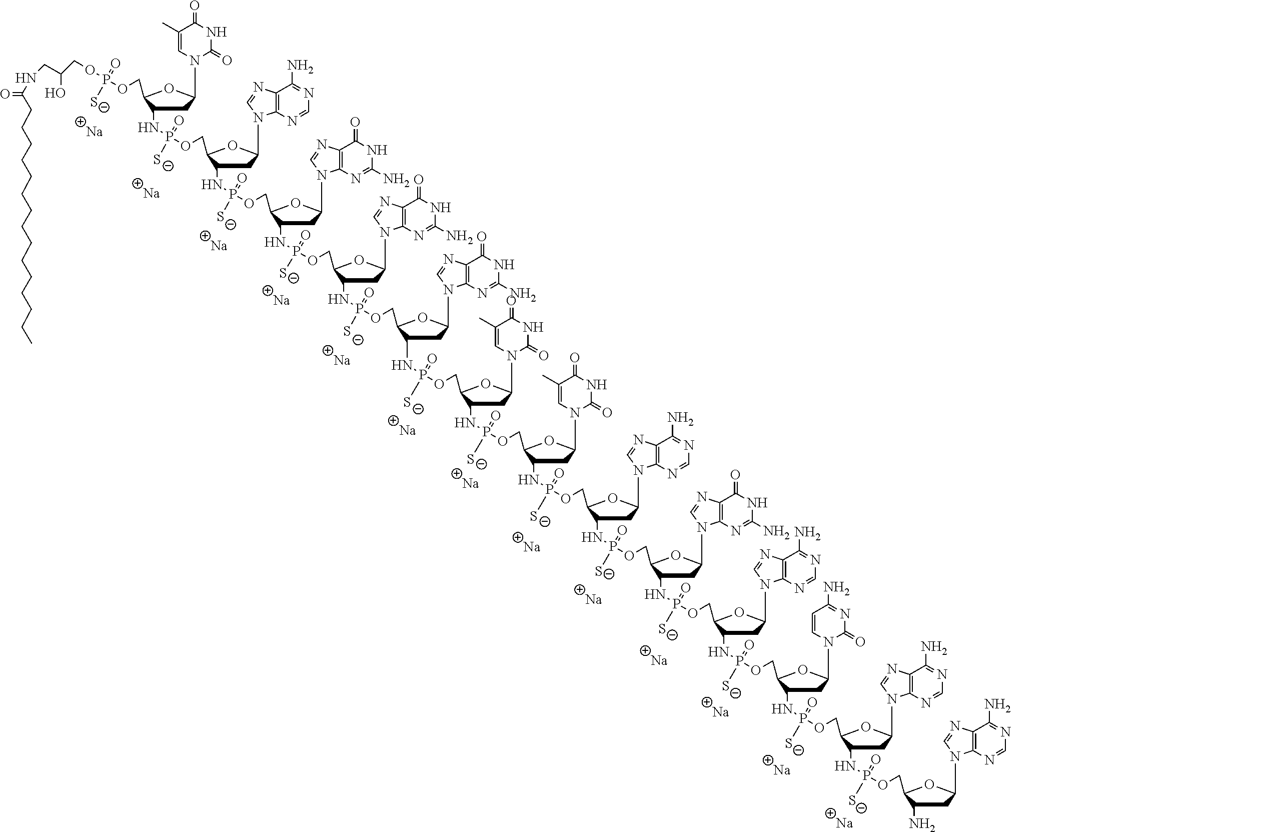

### Imetelstat Sodium

Unless otherwise indicated or clear from the context, references herein to imetelstat also include tautomers thereof and salts thereof, e.g., pharmaceutically acceptable salts. As mentioned, imetelstat sodium refers to the sodium salt of imetelstat. Unless otherwise indicated or clear from the context, references herein to imetelstat or imetelstat sodium also include all tautomers thereof.

Imetelstat and imetelstat sodium can be produced, formulated, or obtained as described elsewhere (see e.g. Asai et al., Cancer Res., 63:3931-3939 (2003), Herbert et al., Oncogene, 24:5262-5268 (2005), and Gryaznov, Chem. Biodivers., 7:477-493 (2010)). Unless otherwise indicated or clear from the context, references herein to imetelstat also include salts thereof. As mentioned, imetelstat sodium refers to the sodium salt of imetelstat.

Imetelstat targets the RNA template of telomerase and inhibits telomerase activity and cell proliferation in various cancer cell lines and tumor xenografts in mice. Phase 1 studies involving patients with breast cancer, non-small-cell lung cancer and other solid tumors, multiple myeloma, or chronic lymphocytic leukemia have provided information on drug pharmacokinetics and pharmacodynamics. A subsequent phase 2 study involving patients with essential thrombocythemia showed platelet-lowering activity accompanied by a significant reduction in JAK2 V617F and CALR mutant allele burdens. Imetelstat sodium is routinely administered intravenously; it is contemplated that in the practice of the subject methods other administration routes also can be used, such as intrathecal administration, intratumoral injection, subcutaneous administration, oral administration, and others. Imetelstat sodium can be administered at doses comparable to those routinely utilized clinically. In certain embodiments, imetelstat sodium is administered as described elsewhere herein.

### F. Pharmaceutical Compositions

For ease of administration, the telomerase inhibitor (e.g., as described herein) may be formulated into various pharmaceutical forms for administration purposes. In some cases, the telomerase inhibitor is administered as a pharmaceutical composition. The carrier or diluent of the pharmaceutical composition must be “acceptable” in the sense of being compatible with the other ingredients of the composition and not deleterious to the recipients thereof. The pharmaceutical composition may be in unitary dosage form suitable, in particular, for administration orally, rectally, percutaneously, by parenteral injection or by inhalation. In some cases, administration can be via intravenous injection. For example, in preparing the composition in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions, for example, may be prepared in which the carrier comprises saline solution, glucose solution or a mixture of saline and glucose solution. Injectable solutions containing the telomerase inhibitor described herein may be formulated in oil for prolonged action. Appropriate oils for this purpose are, for example, peanut oil, sesame oil, cottonseed oil, corn oil, soybean oil, synthetic glycerol esters of long chain fatty acids and mixtures of these and other oils. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired composition. The composition may be administered in various ways, e.g., as a transdermal patch, as a spot-on, as an ointment.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, subcutaneous formulations, and segregated multiples thereof.

In order to enhance the solubility and/or the stability of the drug described herein in pharmaceutical compositions, it can be advantageous to employ α-, β- or γ-cyclodextrins or their derivatives, in particular hydroxyalkyl substituted cyclodextrins, e.g. 2 hydroxypropyl-β-cyclodextrin or sulfobutyl-β-cyclodextrin. Also, co-solvents such as alcohols may improve the solubility and/or the stability of the telomerase inhibitor in pharmaceutical compositions.

Depending on the mode of administration, the pharmaceutical composition may comprise from 0.05 to 99% by weight, such as from 0.1 to 70% by weight, such as from 0.1 to 50% by weight of the telomerase inhibitor described herein, and from 1 to 99.95% by weight, such as from 30 to 99.9% by weight, such as from 50 to 99.9% by weight of a pharmaceutically acceptable carrier, all percentages being based on the total weight of the composition.

### G. Administration and Administration Regimens

The frequency of administration can be any frequency that reduces the severity of a symptom of a MDS (e.g., as described herein) without producing significant toxicity to the subject. For example, the frequency of administration can be from about once every two months to about once a week, alternatively from about once a month to about twice a month, alternatively about once every six weeks, about once every 5 weeks, alternatively about once every 4 weeks, alternatively about once every 3 weeks, alternatively about once every 2 weeks or alternatively about once a week. The frequency of administration can remain constant or can be variable during the duration of treatment. A course of treatment with a composition containing one or more telomerase inhibitors can include rest periods. For example, a composition containing a telomerase inhibitor can be administered weekly over a three-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the MDS and related symptoms may require an increase or decrease in administration frequency.

An effective duration for administering a composition containing a telomerase inhibitor (e.g., imetelstat or imetelstat sodium) can be any duration that reduces the severity of a symptom of a MDS (e.g., as described herein) without producing significant toxicity to the subject. Thus, the effective duration can vary from one month to several months or years (e.g., one month to two years, one month to one year, three months to two years, three months to ten months, or three months to 18 months). In general, the effective duration for the treatment of a MDS can range in duration from two months to twenty months. In some cases, an effective duration can be for as long as an individual subject is alive. Multiple factors can influence the actual effective duration used for a particular treatment. For example, an effective duration can vary with the frequency of administration, effective amount, use of multiple treatment agents, route of administration, and severity of the MDS and related symptoms.

In certain instances, a course of treatment and the severity of one or more symptoms related to a MDS can be monitored. Any method can be used to determine whether or not the severity of a symptom of a MDS is reduced. For example, the severity of a symptom of a MDS (e.g., as described herein) can be assessed using biopsy techniques.

Telomerase inhibitors as used in the subject methods can be administered at any dose that is therapeutically effective, such as doses comparable to those routinely utilized clinically. Specific dose regimens for known and approved anti-cancer agents (e.g., the recommended effective dose) are known to physicians and are given, for example, in the product descriptions found in the PHYSICIANS' DESK REFERENCE, 2003, 57th Ed., Medical Economics Company, Inc., Oradell, N.J.; Goodman & Gilman's THE PHARMACOLOGICAL BASIS OF THERAPEUTICS” 2001, 10th Edition, McGraw-Hill, New York; and/or are available from the Federal Drug Administration and/or are discussed in the medical literature.

In some aspects, the dose of a telomerase inhibitor, imetelstat sodium, administered to the subject is about 1.0 mg/kg to about 13.0 mg/kg. In other aspects, the dose of a telomerase inhibitor is about 4.5 mg/kg to about 11.7 mg/kg or about 6.0 mg/kg to about 11.7 mg/kg or about 6.5 mg/kg to about 11.7 mg/kg. In some embodiments, the dose of a telomerase inhibitor includes at least about any of 4.7 mg/kg, 4.8 mg/kg, 4.9 mg/kg, 5.0 mg/kg, 5.5 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 10.1 mg/kg, 10.2 mg/kg, 10.3 mg/kg, 10.4 mg/kg, 10.5 mg/kg, 10.6 mg/kg, 10.7 mg/kg, 10.8 mg/kg, 10.9 mg/kg, 11 mg/kg, 11.1 mg/kg, 11.2 mg/kg, 11.3 mg/kg, 11.4 mg/kg, 11.5 mg/kg, 11.6 mg/kg, 11.7 mg/kg, 11.8 mg/kg, 11.9 mg/kg, 12 mg/kg, 12.1 mg/kg, 12.2 mg/kg, 12.3 mg/kg, 12.4 mg/kg, 12.5 mg/kg, 12.6 mg/kg, 12.7 mg/kg, 12.8 mg/kg, 12.9 mg/kg, or 13 mg/kg.

In some embodiments, the effective amount of a telomerase inhibitor administered to the individual includes at least about any of 1 mg/kg, 2.5 mg/kg, 3.5 mg/kg, 4.7 mg/kg, 5 mg/kg, 6.0 mg/kg, 6.5 mg/kg, 7.5 mg/kg, 9.4 mg/kg, 10 mg/kg, 15 mg/kg, or 20 mg/kg. In some embodiments, the effective amount of a telomerase inhibitor administered to the individual is about any of 1 mg/kg, 2.5 mg/kg, 3.5 mg/kg, 5 mg/kg, 6.5 mg/kg, 7.5 mg/kg, 9.4 mg/kg, 10 mg/kg, 15 mg/kg, or 20 mg/kg. In various embodiments, the effective amount of a telomerase inhibitor administered to the individual includes less than about any of 350 mg/kg, 300 mg/kg, 250 mg/kg, 200 mg/kg, 150 mg/kg, 100 mg/kg, 50 mg/kg, 30 mg/kg, 25 mg/kg, 20 mg/kg, 10 mg/kg, 7.5 mg/kg, 6.5 mg/kg, 5 mg/kg, 3.5 mg/kg, 2.5 mg/kg, 1 mg/kg, or 0.5 mg/kg of a telomerase inhibitor.

Exemplary dosing frequencies for the pharmaceutical composition including a telomerase inhibitor include, but are not limited to, daily; every other day; twice per week; three times per week; weekly without break; weekly, three out of four weeks; once every three weeks; once every two weeks; weekly, two out of three weeks. In some embodiments, the pharmaceutical composition is administered about once every week, once every 2 weeks, once every 3 weeks, once every 4 weeks, once every 5 weeks, once every 6 weeks, once every 7 weeks or once every 8 weeks. In some embodiments, the composition is administered at least about any of 1×, 2×, 3×, 4×, 5×, 6×, or 7× (i.e., daily) a week, or three times daily, two times daily. In some embodiments, the intervals between each administration are less than about any of 6 months, 3 months, 1 month, 20 days, 15 days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

Telomerase inhibitors such as imetelstat (e.g., imetelstat sodium) can be administered using any appropriate method. For example, telomerase inhibitors such as imetelstat (e.g., imetelstat sodium) can be administered intravenously once every 4 weeks over a period of time (e.g., one, two, three, four, or five hours). In some embodiments, imetelstat is administered intravenously once weekly over a period of about 2 hours at 7-10 mg/kg. In certain embodiments, imetelstat is administered intravenously once every 3 weeks over a period of about 2 hours at 2.5-7 mg/kg. In an embodiment, imetelstat is administered intravenously for a period of about 2 hours once every 4 weeks at 0.5-5 mg/kg. In an embodiment, imetelstat is administered intravenously once every 3 weeks over a period of about 2 hours at about 2.5-10 mg/kg. Alternatively, imetelstat is administered intravenously for a period of about 2 hours once every 4 weeks at about 0.5-9.4 mg/kg.

In certain embodiments of the method, imetelstat is administered for 1, 2, 3, 4, 5, 6, 7, 8 or more than 8 dosage cycles, each cycle comprising: intravenous administration of about 7-10 mg/kg imetelstat once every four weeks, intravenous administration of about 7-10 mg/kg imetelstat once weekly for four weeks, intravenous administration of about 2.5-10 mg/kg imetelstat once every three weeks, or intravenous administration of about 0.5-9.4 mg/kg imetelstat once every four weeks. In certain instances, each dosage cycle comprises intravenous administration of about 7-10 mg/kg imetelstat once every four weeks. In some cases, each dosage cycle comprises intravenous administration of about 7.5 mg/kg imetelstat about once every four weeks.

In one embodiment of the invention, imetelstat is administered intravenously at a dosage of about 7-10 mg/kg imetelstat once every four weeks following premedication with an antihistamine, corticosteroid, or both. In other embodiments, imetelstat is administered intravenously at a dosage of about 7.5 mg/kg, alternatively from about 7.0 mg/kg to about 7.7 mg/kg, imetelstat once every four weeks following premedication with an antihistamine, corticosteroid, or both.

In certain embodiments, imetelstat is administrated at a dosage of about 7.5 mg/kg, alternatively from about 7.0 mg/kg to about 7.7 mg/kg, once every four weeks for at least three cycles and then the dosage is increased. In certain embodiments, the dosage of imetelstat may be increased to about 9.4 mg/kg, alternatively from about 8.8 mg/kg to about 9.6 kg/mg, provided ANC and platelet nadir have not dropped between about 1.5×10⁹/L and about 75×10⁹/L, respectively, and there is no grade ≥3 non-hematological toxicity.

In one embodiment of the invention, imetelstat sodium is administered intravenously at a dosage of about 4.5-11.7 mg/kg, such as of about 7-10 mg/kg imetelstat sodium, once every four weeks. In other embodiments, imetelstat sodium is administered intravenously at a dosage of about 7.5 mg/kg, alternatively from about 7.0 mg/kg to about 7.7 mg/kg, imetelstat sodium once every four weeks.

It will be appreciated that treatment for cancer sometimes involves multiple “rounds” or “cycles” of administration of a drug, where each cycle comprises administration of the drug one or more times according to a specified schedule (e.g., every three weeks for three consecutive days; once per week; etc.). For example, anti-cancer drugs can be administered for from 1 to 8 cycles, or for a longer period. When more than one drug (e.g., two-drugs) is administered to a subject, each can be administered according to its own schedule (e.g., weekly; once every three weeks; etc.). It will be clear that administration of drugs, even those administered with different periodicity, can be coordinated so that both drugs are administered on the same day at least some of the time or, alternatively, so the drugs are administered on consecutive days at least some of the time.

As is understood in the art, treatment with cancer therapeutic drugs can be suspended temporarily if toxicity is observed, or for the convenience of the patient, without departing from the scope of the invention, and then resumed.

In certain embodiments, the invention relates to a telomerase inhibitor for use in a method of treating MDS the method comprising administering to a subject in need thereof an effective amount of a telomerase inhibitor; wherein the subject is naive to treatment with an agent selected from a HMA and lenalidomide. In other embodiments, the invention relates to a telomerase inhibitor for use in a method of treating MDS, the method comprising administering to a subject in need thereof an effective amount of a telomerase inhibitor; wherein the subject is naive to treatment with an agent selected from a HMA, lenalidomide, and combination thereof.

In certain embodiments, the invention relates to a telomerase inhibitor for use in a method as defined in any of the other embodiments.

### H. Exemplary Embodiments

Exemplary embodiments of the methods of treating MDS of the invention, which involve administering to a subject in need thereof an effective amount of a telomerase inhibitor whereby the subject is naive to treatment with an agent selected from a hypomethylating agent (HMA) and lenalidomide are shown in Table 1 below.

Exemplary embodiments include using any of the telomerase inhibitors in Table 1 to treat any one of the types of MDS shown in Table 1 in any one of the subjects shown in Table 1, whereby the subject is naïve to any one of the treatments shown in Table 1. In certain embodiments, one of the administration regimens described in Table 1 is used. In other embodiments, the methods may be used to treat any one of the types of MDS shown in Table 1 in any one of subjects shown in Table 1 using imetelstat (imetelstat sodium), whereby the subject is naïve to any one of the treatments shown in Table 1. When imetelstat, e.g., imetelstat sodium is used, any of the administration regimens shown in Table 1 may be used.

TABLE 1
Exemplary embodiments of the invention

| | |
|---|---|
| Type of MDS | MDS |
| | Relapsed or refractory MDS |
| | Relapsed MDS |
| | Refractory MDS |
| | MDS relapsed or refractory to ESA) (e.g. erythropoietin-alpha, |
| | erythropoietin-beta, darbepoetin, or combination thereof). |
| Subject | low or intermediate-1 IPSS risk MDS subject |
| | low or intermediate-1 IPSS risk MDS subject and transfusion |
| | dependent |
| | low or intermediate-1 IPSS risk MDS subject and transfusion |
| | dependent with a transfusion requirement of about 4 units or more |
| | during the 8 weeks prior to the administration of the telomerase |
| | inhibitor |
| | transfusion dependent e.g. transfusion dependent with a transfusion |
| | requirement of about 4 units or more during the 8 weeks prior to the |
| | administration of the telomerase inhibitor |
| | low or intermediate-1 IPSS risk MDS subject and non-del5q |
| | low or intermediate-1 IPSS risk MDS subject, non-de15q, and |
| | transfusion dependent |
| | low or intermediate-1 IPSS risk MDS subject, non-del5q, and |
| | transfusion dependent with a transfusion requirement of about 4 |
| | units or more during the 8 weeks prior to the administration of the |
| | telomerase inhibitor |
| | non-del5q |
| | non-del5q and transfusion dependent |
| | non-del5q and transfusion dependent with a transfusion requirement |
| | of about 4 units or more during the 8 weeks prior to the |
| | administration of the telomerase inhibitor |
| Subject naive to | Hypomethylating agent (e.g. decitabine or azacitidine) |
| treatment with | Hypomethylating agent (e.g. decitabine or azacitidine) and |
| | lenalidomide |
| | decitabine |
| | azacitidine |
| | azacitidine and decitabine |
| | lenalidomide |
| | lenalidomide and decitabine |
| | lenalidomide and azacitidine |
| | lenalidomide, azacitidine, and decitabine |
| Telomerase | Any suitable inhibitor or |
| inhibitor | imetelstat (imetelstat sodium) |
| Administration | Administration of telomerase inhibitor for 1, 2, 3, 4, 5, 6, 7, 8 or |
| | more than 8 dosage cycles |
| | Administration of imetelstat (imetelstat sodium) for 1, 2, 3, 4, 5, 6, |
| | 7, 8 or more than 8 dosage cycles, each cycle comprising: (a) |
| | intravenous administration of about 7-10 mg/kg imetelstat once |
| | every four weeks; (b) intravenous administration of about 7-10 |
| | mg/kg imetelstat once weekly for four weeks; (c) intravenous |
| | administration of about 2.5-10 mg/kg imetelstat once every three |
| | weeks; or (d) intravenous administration of about 0.5-9.4 mg/kg |
| | imetelstat once every four weeks. |
| | Administration of imetelstat (imetelstat sodium) for 1, 2, 3, 4, 5, 6, |
| | 7, 8 or more than 8 dosage cycles, each cycle comprises intravenous |
| | administration of about 4.5-11.7 mg/kg of imetelstat (imetelstat |
| | sodium) once every four weeks |
| | Administration of imetelstat (imetelstat sodium) for 1, 2, 3, 4, 5, 6, |
| | 7, 8 or more than 8 dosage cycles, each cycle comprises intravenous |
| | administration of about 7-10 mg/kg of imetelstat (imetelstat sodium) |
| | once every four weeks |
| | Administration of imetelstat (imetelstat sodium) for 1, 2, 3, 4, 5, 6, |
| | 7, 8 or more than 8 dosage cycles, each cycle comprises intravenous |
| | administration of about 7.5 mg/kg imetelstat of imetelstat (imetelstat |
| | sodium) once every four weeks |
| Pharmacodynamics | Reduction of 25% or more, such as 30% or more, 35% or more, 40% |
| | or more, 45% or more, 50% or more, 55% or more, 60% or more, |
| | 65% or more, 70% or more, 75% or more, 80% or more, 85% or |
| | more, 90% or more, or 95% or more in the VAF for one or more of |
| | the following genes: SF3B1, TET2, DNMT3A and ASXL1. |

The following examples are offered by way of illustration and not by way of limitation.

### EXAMPLES

Example 1: Efficacy and Safety of Imetelstat in Transfusion-Dependent (TD) Patients with International Prognostic Scoring System (IPSS) Low/Intermediate-1 Risk Myelodysplastic Syndromes that are Relapsed or Refractory to Erythropoiesis-Stimulating Agent (ESA) Treatment

### Introduction

Global study of imetelstat sodium in RBC TD patients, ESA-relapsed or refractory or EPO (Erythropoietin) >500 mU/ml, and IPSS Low- or Intermediate 1-Risk MDS. This example provides safety and efficacy findings from 177 patients. The results suggest improved efficacy of imetelstat among these patients.

### Methods

### Trial Design and Oversight

A randomized, double-blind, placebo-controlled trial was conducted at 77 sites in 17 countries. The trial was conducted according to institutional and CONSORT (Consolidated Standards of Reporting Trials) guidelines and laws of applicable authorities. Institutional review boards or ethics committees at each site approved the protocol. All patients were provided with written informed consent. The trial was designed in collaboration with the external steering committee; and an independent data and safety monitoring board monitored the trial. The sponsor provided guidance in trial design development and data collection assessment.

### Patients

Eligible patients were age 18 years or older, had MDS confirmed by BM aspirate and biopsy within 12 weeks prior to randomization by an independent central laboratory; low or intermediate-1 risk disease by IPSS; were RBC-TD requiring ≥4 units over an 8-week period during 16 weeks prior to randomization; had MDS that was relapsed, refractory, or ineligible (due to endogenous erythropoietin level >500 mU/mL) for ESAs; had not received treatment with lenalidomide or a hypomethylating agent; and were non-del(5q).

### Trial Design

Patients were randomly assigned (based on a computer-generated schedule) in a 2:1 ratio to receive imetelstat or placebo, administered intravenously every 4 weeks until treatment discontinuation; crossover was not allowed. Randomization was stratified by prior RBC transfusion burden (≥4 to ≤6 or >6 units over an 8-week period during 16 weeks prior to randomization) and IPSS risk group (low or intermediate-1 risk). The starting dose of imetelstat was 7.5 mg/kg every four weeks. If a patient developed grade 3 or 4 hematologic and nonhematologic toxicities, dosing was held and, if necessary, reduced (Tables 2-4).

TABLE 2
Imetelstat dosing modification procedure.

| | | Dose Regimen |
|---|---|---|
| Dose Level | | (Imetelstat or Equivalent Volume of Placebo) |
| 0 | Starting dose | 7.5 mg/kg IV every 4 weeks |
| −1 | Dose reduction no. 1 | 6.0 mg/kg IV every 4 weeks |
| −2 | Dose reduction no. 2 | 4.7 mg/kg IV every 4 weeks |

IV denotes intravenous.

TABLE 3
Dose modifications for Grade 3 and 4
nonhematologic/nonhepatic toxicities.

| Occurrence | Action |
|---|---|
| First | Hold study drug until recovery to Grade ≤1 or baseline; |
| | restart at 1 dose level lower |
| Second | Hold study drug until recovery to Grade ≤1 or baseline; |
| | restart at 1 dose level lower |
| Third | Discontinue study drug |

TABLE 4
Dose modification procedure for liver function test elevation and hepatic toxicity.

| | |
|---|---|
| Occurrence | Action |

Dose Modifications for Grade 3 Hematologic Toxicities

| | |
|---|---|
| First | Hold study drug until recovery to ANC 1.0 × 10⁹/L and platelets |
| | 50 × 10⁹/L*; may restart at most recent dose level |
| Second | Hold study drug until recovery to ANC 1.0 × 10⁹/L and platelets |
| | 50 × 10⁹/L*; restart at 1 dose level lower |
| Third | Hold study drug until recovery to ANC 1.0 × 10⁹/L and platelets |
| | 50 × 10⁹/L*; restart at 1 dose level lower |
| Fourth | Discontinue study drug |

Dose Modifications for Grade 4 Hematologic Toxicities

| | |
|---|---|
| First | Hold study drug until recovery to ANC 1.0 × 10⁹/L and platelets |
| | 50 × 10⁹/L*; restart at 1 dose level lower |
| Second | Hold study drug until recovery to ANC 1.0 × 10⁹/L and platelets |
| | 50 × 10⁹/L*; restart at 1 dose level lower |
| Third | Discontinue study drug |

Dose Modifications for Liver Function Test Elevations and Hepatic Adverse Events

| | |
|---|---|
| Grade 3 or 4 AST, ALT, | HOLD study drug and report as adverse event of interest (AEI) |
| or bilirubin For ALT or | Determine relatedness to study drug (i.e., extensive investigation |
| AST >8 × ULN or | to determine an alternative likely cause(s) for the event) |
| baseline, contact sponsor | If considered at least possibly related, after recovery to baseline, |
| and consider | restart study drug at one dose level lower. |
| discontinuing drug | If not related, after recovery to baseline, restart study drug |
| altogether | without dose reduction. |
| | If the event recurs and is related, study drug should be |
| | discontinued |
| Grade 3 or 4 hepatic | HOLD study drug and report as AEI |
| adverse event | Determine relatedness to study drug (i.e., extensive investigation‡ |
| | to determine an alternative likely cause(s) for the event) |
| | If considered at least possibly related, after recovery to baseline, |
| | restart study drug at one dose level lower. |
| | If not related, after recovery to baseline, restart study drug |
| | without dose reduction. |
| | If the event recurs and is related, study drug should be |
| | discontinued |
| Grade ≥2 AST or ALT | HOLD study drug and report as AEI |
| with | Determine relatedness to study drug (i.e., extensive investigation‡ |
| concomitant Grade ≥2 | to determine an alternative likely cause(s) for the event) |
| bilirubin* | If considered at least possibly related, discontinue study drug |
| *Patients with Grade 2 | If not related, after recovery to baseline, study drug can be |
| bilirubin at study entry | restarted at same dose |
| should have worsening | If upon rechallenge the event recurs, study drug should be |
| bilirubin with | discontinued |
| concomitant Grade 2 | |
| AST or ALT elevation | |

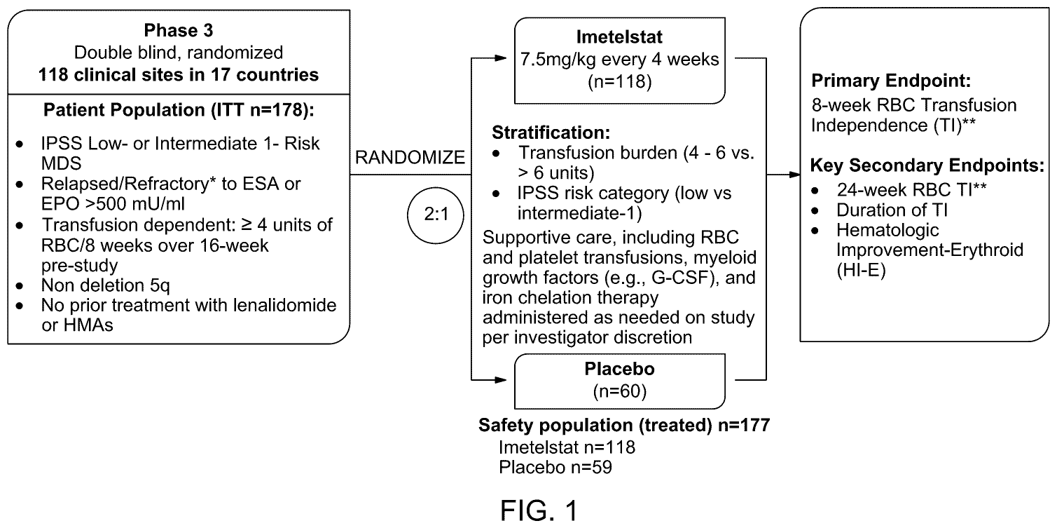

Transfusion status was assessed at every planned and unscheduled visit. Disease assessment occurred every 12 weeks after first dose up to week 72, and thereafter every 24 weeks until treatment discontinuation, and within 30 days of last dose for patients who discontinued, whenever possible. Patients who had unacceptable toxicities, disease progression, withdrew consent, or met discontinuation criteria, stopped treatment. After treatment discontinuation, patients were assessed every 12 to 16 weeks, and followed up for transfusion and disease status every 4 to 6 weeks. (The trial design is shown in FIG. 1).

### End Point Measures

The primary end point was rate of RBC-TI for 8 consecutive weeks or longer. Key secondary end points included rate of 24-week TI, duration of TI, and the rate of hematologic response (hematologic improvement-erythroid [HI-E], based on modified International Working Group [IWG] 2006 criteria, and HI-E per revised IWG 2018). Additional secondary end points included relative change in number of RBC transfusions received by patients, relative change in hemoglobin, and safety. The changes in mutational status and in patient-reported fatigue over time (assessed by the Functional Assessment of Chronic Illness Therapy [FACIT] Fatigue Scale) were exploratory end points. Subgroup analyses were performed including baseline presence or absence of RS, prior RBC transfusion burden (≥4 to ≤6 or >6 RBC units/8-weeks during a 16-week period prior to randomization), and low or intermediate-1 IPSS risk categories. Subgroup analyses were also performed, including baseline mutation status as well as telomerase activity (TA), telomere lengths (TL), and human telomerase reverse transcriptase (hTERT) levels. Peripheral blood hematology, bone marrow evaluation, mutation assessment, and cytogenetic evaluation were performed centrally and responses assessed by an independent review committee (IRC). Rate of 1-year RBC-TI was reported as a post hoc analysis.

### Statistical Analysis

The data cutoff date for this primary analysis was 1 year after the last patient was randomized. All the efficacy analyses for primary and key secondary end points were conducted in the intent-to-treat population. The primary hypothesis of this trial was that imetelstat will significantly improve the rate of RBC-TI as compared with placebo in patients with RBC-TD lower risk MDS. The percentages of patients with a response for the two treatment groups were compared with a stratified Cochran-Mantel-Haenszel test at a two-sided significance level of 0.05 adjusting for stratification factors, prior RBC transfusion burden, and IPSS risk category low or intermediate-1. Multiplicity was accounted for by a sequential gatekeeping approach for the primary and key secondary end points, which were tested sequentially in the prespecified order of 8-week TI, followed by 24-week TI. Durations of RBC-TI were calculated by Kaplan-Meier method. Safety analyses were conducted in the safety population (all patients who received ≥1 dose of trial medication).

The trial design is summarized in the following paragraphs:

Eligibility: The eligibility requirements for the study were as follows:
IPSS Low- or Intermediate 1-Risk MDS
Relapsed or Refractory* to ESA or EPO >500 mU/ml. (*Received at least 8 weeks of ESA treatment (epoetin alfa≥40,000U, epoetin beta≥30,000U or darbepoetin alfa 150 mcg or equivalent per week) without Hgb rise≥1.5 g/dL or decreased RBC transfusion requirement≥4 units/8 weeks or transfusion dependence or reduction in Hgb by ≥1.5 g/dL after hematologic improvement from ≥8 weeks of ESA treatment.
Transfusion dependent: ≥4 units of RBC/8 weeks over 16-week pre-study
Non deletion 5q
No prior treatment with lenalidomide or HMAs.

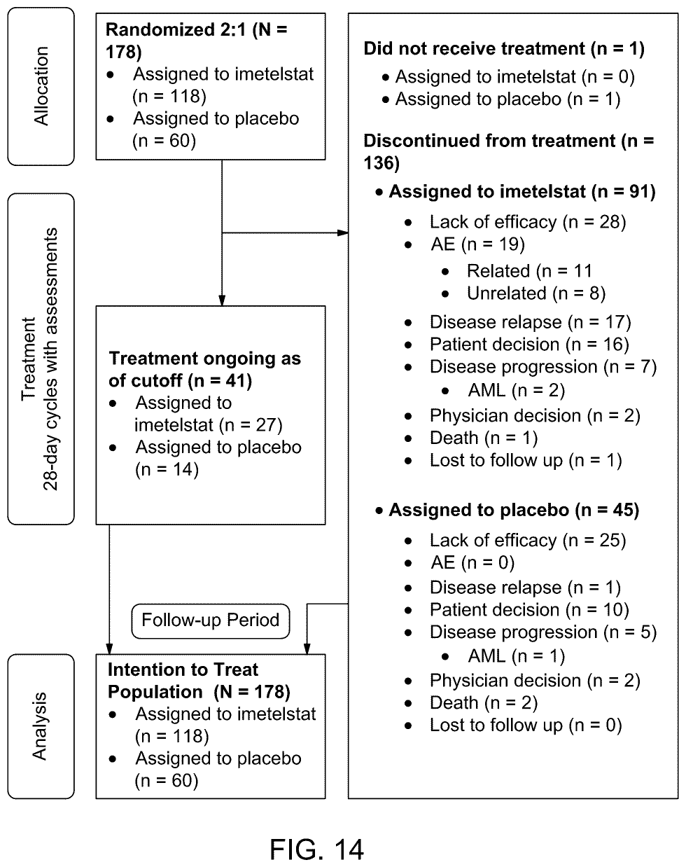

178 patients were randomized in a 2:1 ratio between imetelstat and placebo groups: 118 patients received imetelstat and 59 patients received placebo (FIG. 14.). Details of the disposition of patients at data cutoff are also shown in FIG. 14 and includes 27 patients (23%) in the imetelstat group and 14 patients (24%) in the placebo group that were still on treatment at data cutoff.

### Stratification:

Transfusion burden (4-6 vs. >6 units)
IPSS risk category (low vs intermediate-1)

Treatment: In the patients from imetelstat treatment group, imetelstat sodium was administered as a 2-hour IV infusion every 4 weeks at 7.5 mg/kg. Supportive care, including RBC and platelet transfusions, myeloid growth factors (e.g., G-CSF (granulocyte colony stimulating factor)), and iron chelation therapy was administered as needed on study per investigator discretion. Control group received corresponding treatment without imetelstat.

End points and Analysis:
Primary end point: 8-week RBC TI. Proportion of patients without any RBC transfusion for at least eight consecutive weeks since entry to the trial (8-week TI).
Key secondary end points:
   24-week RBC TI; proportion of patients without any RBC transfusion for at least 24 consecutive weeks since entry to the trial (24-week TI).
   Duration of TI;
   Hematologic improvement-Erythroid (HI-E).

### Results

### Key Trial Metrics

Balanced time on study and treatment time between two arms is shown in Table 5 below:

TABLE 5

| | Imetelstat | | Placebo | |
|---|---|---|---|---|
| | (n = 118) | | (n = 60) | |
| Median time on study, months (range) | 19.5 | (1.4-36.2) | 17.5 | (0.7-34.3) |
| Median time on treatment, months | 7.8 | (0.03-32.5) | 6.5 | (0.03-26.7) |
| (range) | | | | |
| Median time on treatment for 8-week TI | 17.2 | (1.8-32.5) | 15.0 | (4.1-25.8) |
| responders, months (range) | | | | |
| Median treatment, cycles (range) | 8 | (1-34) | 8 | (1-30) |
| Median treatment cycles for 8-week TI | 18 | (3-34) | 17 | (3-29) |

responders, months (range)

8-week TI = proportion of patients without any RBC transfusion for at least eight consecutive
weeks since entry to the trial

Baseline demographics and disease characteristics were comparable between both arms as shown in Table 6A below. Additional baseline characteristics are shown in Table 6B below.

TABLE 6A

| | Imetelstat | | Placebo | | Total | |
|---|---|---|---|---|---|---|
| Characteristic | (N = 118) | | (N = 60) | | (N = 178) | |
| Median age (range) - years | 72 | (44-87) | 73 | (39-85) | 72 | (39-87) |
| Male sex - No. of patients (%) | 71 | (60) | 40 | (66) | 111 | (62) |
| Median time since original | 3.5 | (0.1-26.7) | 2.8 | (0.2-25.7) | 3.3 | (0.1-26.7) |
| diagnosis of MDS (range) - | | | | | | |
| years | | | | | | |
| Ring sideroblast status - No. of | | | | | | |
| patients (%) | | | | | | |
| With ring sideroblasts | 73 | (62) | 37 | (62) | 110 | (62) |
| Without ring sideroblasts | 44 | (37) | 23 | (38) | 67 | (38) |
| IPSS category† | | | | | | |
| Low | 80 | (68) | 39 | (65) | 119 | (67) |
| Intermediate-1 | 38 | (32) | 21 | (35) | 59 | (33) |
| IPSS-R prognostic risk category - | | | | | | |
| No. of patients (%)‡ | | | | | | |
| Very Low | 3 | (3) | 2 | (3) | 5 | (3) |
| Low | 87 | (74) | 46 | (77) | 133 | (75) |
| Intermediate | 20 | (17) | 8 | (13) | 28 | (16) |
| High | 1 | (1) | 0 | | 1 | (1) |
| Very High | 0 | | 0 | | 0 | |
| Median prior red blood cell | 6 | (4-33) | 6 | (4-13) | 6 | (4-33) |
| transfusion burden - No. red | | | | | | |
| blood cell units/8 weeks | | | | | | |
| (range) | | | | | | |
| Prior red blood cell transfusion | | | | | | |
| burden† - No. of patients (%) | | | | | | |
| ≥4 to ≤6 units | 62 | (53) | 33 | (55) | 95 | (53) |
| >6 units | 56 | (48) | 27 | (45) | 83 | (47) |
| Median pretreatment | 7.92 | (5.3-10.1) | 7.8 | (6.1-9.2) | 7.86 | (5.3-10.1) |
| hemoglobin level - g/dL | | | | | | |
| (range)‡ | | | | | | |
| Prior erythropoiesis stimulating | 108 | (92) | 52 | (87) | 160 | (90) |
| agents use - No. of patients | | | | | | |
| (%) | | | | | | |
| Median serum erythropoietin | 174.9 | (6.0-4460.0) | 277.0 | (16.9-5514.0) | 184.1 | (6.0-5514.0) |
| level - mU/mL (range) | | | | | | |
| ≤500 mU/mL, n (%) | 87 | (74) | 36 | (60) | 123 | (69) |
| >500 mU/mL, n (%) | 26 | (22) | 22 | (37) | 48 | (27) |
| Prior luspatercept - No. of | 7 | (6) | 4 | (7) | 11 | (6) |

Patients (%)§

*Percentages may not total 100 because of rounding. CRF denotes case report form, IPSS denotes International Prognostic Scoring System, MDS denotes myelodysplastic syndrome, WHO denotes World Health Organization, and wk denotes week.
†Stratification factors.
‡Pretreatment hemoglobin is defined as the average of all hemoglobin in the 8 weeks prior to the first dose date, excluding values that were within 14 days after transfusion; thus, considered to be influenced by transfusion.
§Insufficient number of patients previously treated with luspatercept to draw conclusions about the effect of imetelstat treatment in such patients. Six of seven patients with ESA therapy failure and treated with luspatercept in the imetelstat group had a reduction in transfusion burden; however, none achieved RBC-TI response.
IPSS = International Prognostic Scoring System; RBC = red blood cell; RS+ = ring sideroblast positive; RS- = ring sideroblast negative.

TABLE 6B
Additional baseline characteristics.

| | Imetelstat | | Placebo | | Total | |
|---|---|---|---|---|---|---|
| Characteristic | (N = 118) | | (N = 60) | | (N = 178) | |
| Median local neutrophils - ×10⁹/L | 2.630 | (0.47-26.84) | 2.700 | (0.90-8.82) | 2.630 | (0.47-26.84) |
| (range) | | | | | | |
| Median local platelets - x10⁹/L | 230.0 | (41-630) | 229.5 | (77-446) | 230.0 | (41-630) |
| (range) | | | | | | |
| | Imetelstat | | Placebo | | Total | |
| IPSS-M n (%) | (N = 110) | | (N = 55) | | (N = 165) | |
| Very low | 4 | (4) | 0 | | 4 | (2) |
| Low | 68 | (62) | 34 | (62) | 102 | (62) |
| Moderate low | 26 | (24) | 11 | (20) | 37 | (22) |
| Moderate high | 7 | (6) | 7 | (13) | 14 | (8) |
| High | 4 | (4) | 3 | (6) | 7 | (4) |

| | | | | | |
|---|---|---|---|---|---|
| Very high | 1 | (1) | 0 | 1 | (1) |

Overall, 178 patients were enrolled; 118 patients were randomly assigned to receive imetelstat and 60 to receive placebo (FIG. 14). After a median of 18 months (range, 0.7 to 36.2) of follow-up, patients received a median of 8 (range, 1 to 34) treatment cycles of imetelstat and 8 treatment cycles of placebo (range, 1 to 30). The median duration of treatment was 33.9 weeks (range, 0.1 to 141.1) on imetelstat and 28.3 weeks (range, 0.1 to 116) weeks on placebo. The mean relative dose intensity with respect to the starting dosage of 7.5 mg/kg/cycle was 90% (range, 64 to 103) in the imetelstat group and 98% (range, 72 to 105) in the placebo group. Details of the disposition of patients at data cutoff are shown in FIG. 14 and includes 27 patients (23%) in the imetelstat group and 14 patients (24%) in the placebo group that were still on treatment at data cutoff.

The baseline characteristics of patients were well balanced between the groups (Table 6A). Additional baseline characteristics are shown in Table 6B. Overall, the median age of patients was 72 years (range, 39 to 87 years) and 62% of the patients were male. Median pretreatment hemoglobin was 7.9 g/dL (range, 5.3 to 10.1) and median baseline serum erythropoietin was 184.1 mU/mL (range, 6 to 5514.0). For baseline IPSS risk category, 67% of patients had low risk and 33% had intermediate-1 risk; 62% of patients had MDS-RS. Patients were heavily transfused at baseline with a median prior RBC transfusion burden of 6.0 units/8 weeks (range, 4 to 33) and the prior transfusion burden was >6 units/8 weeks in 47% of patients. Overall, 90% of patients previously received ESA, and 6% previously received luspatercept.

Patient disposition after 18 months median follow up is shown in Table 7 below:

TABLE 7

| | |
|---|---|
| Imetelstat | Placebo |
| (n = 118) | (n = 59) |

| Treatment ongoing, n (%) | 27 | (22.9) | 14 | (23.7) |
|---|---|---|---|---|
| Treatment discontinued, n (%) | 91 | (77.1) | 45 | (76.3) |
| Lack of efficacy | 28 | (23.7) | 25 | (42.4) |
| Adverse event | 19 | (16.1) | 0 | |
| Cytopenias | 11 | (9.3) | 0 | |
| Unrelated | 8 | (6.8) | 0 | |
| Disease relapse after initial | 17 | (14.4) | 1 | (1.7) |

response on study

| | | | | |
|---|---|---|---|---|
| Patient decision | 16 | (13.6) | 10 | (16.9) |
| Progressive disease | 7 | (5.9) | 5 | (8.5) |
| AML progression | 2 | (1.7) | 1 | (1.7) |
| Investigator decision | 2 | (1.7) | 2 | (3.4) |
| Death* | 1 | (0.8) | 2 | (3.4) |

| | | | |
|---|---|---|---|
| Lost to follow up | 1 | (0.8) | 0 |

*Imetelstat treated patient death: sepsis not related to drug after ~2-year treatment duration Placebo treated patient deaths: (1) COVID and (1) heart valve issue.
AML = acute myeloid leukemia

### Primary End Point: RBC-TI for 8 Consecutive Weeks or Longer

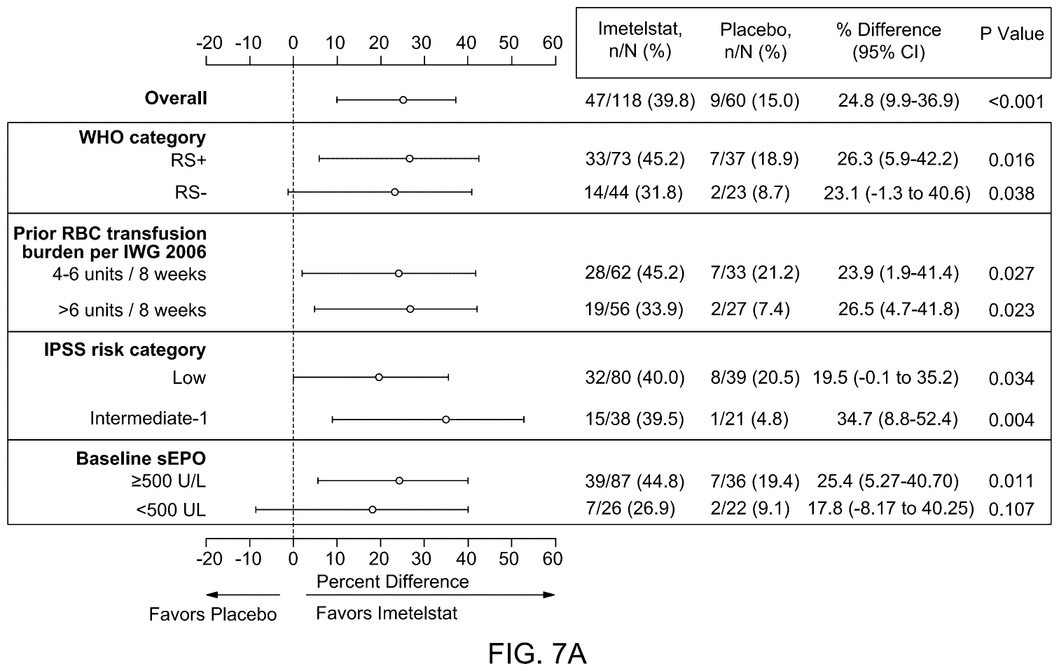

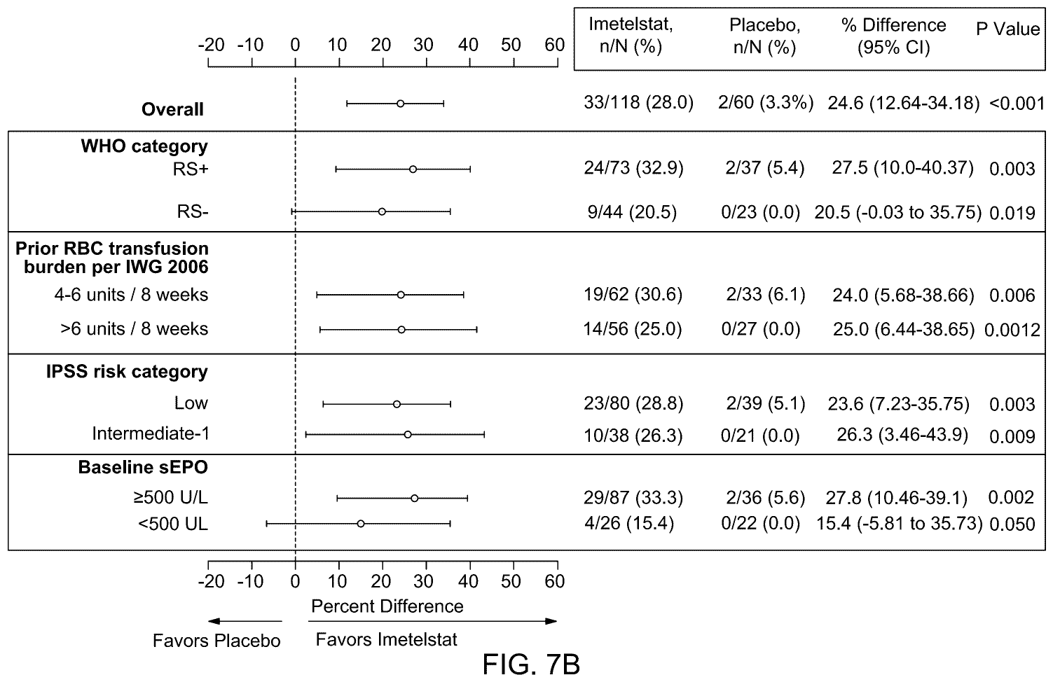

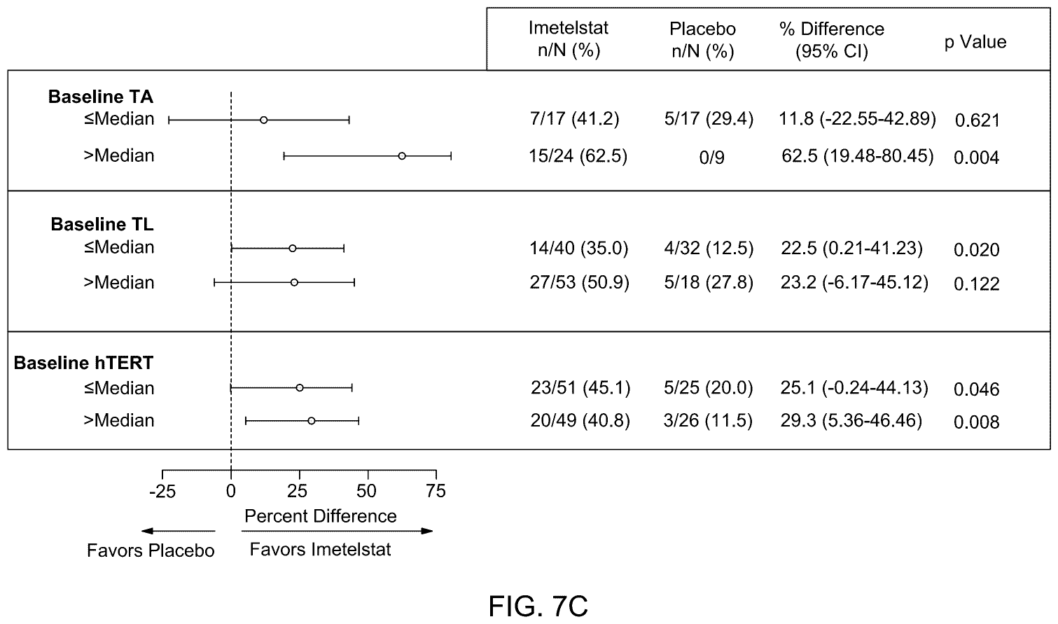

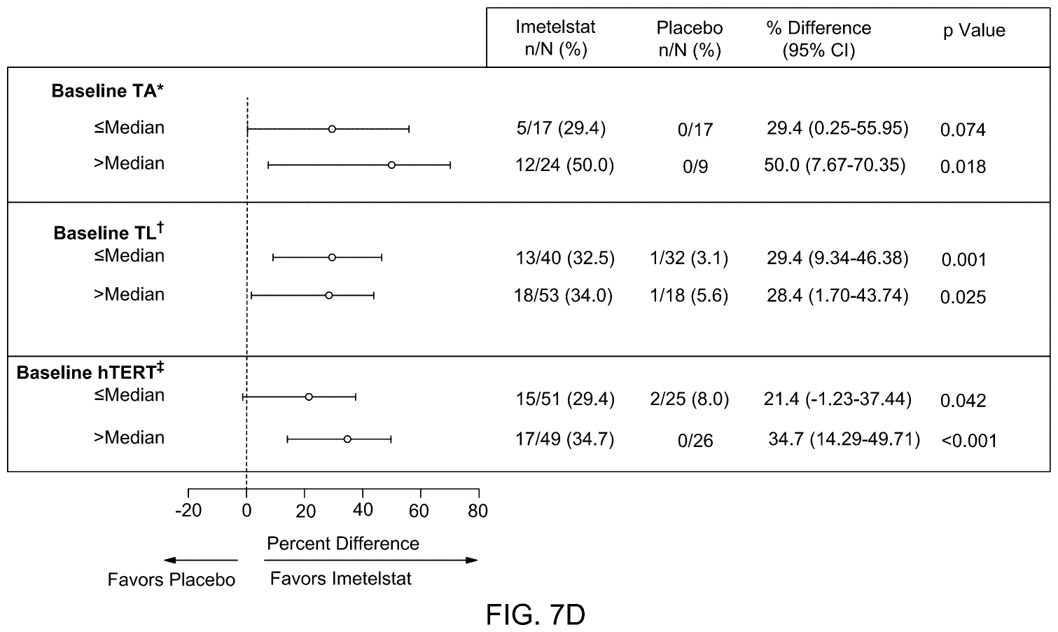

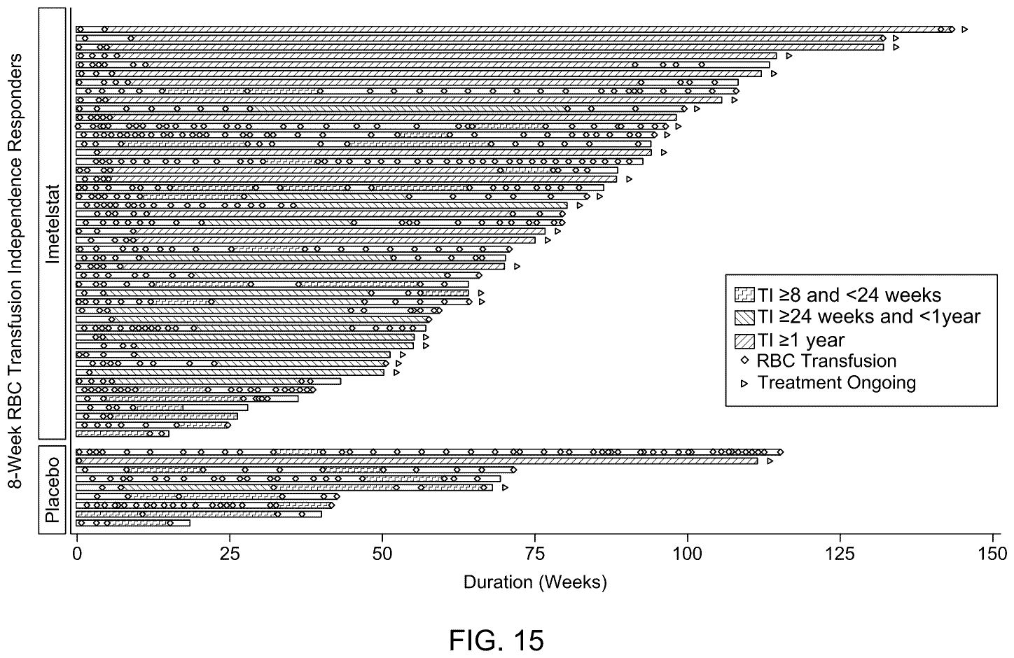

Forty-seven patients (39.8%) in the imetelstat group achieved RBC-TI for at least 8 weeks, compared with 9 patients (15.0%) in the placebo group (P<0.001; FIG. 1A). RBC-TI was continuous and sustained with imetelstat treatment; 83% of 8-week RBC-TI responders had a single continuous RBC-TI period (FIG. 15). The primary end point was achieved in patients with and without RS, 45% (33 of 73) and 32% of patients (14 of 44) in the imetelstat group, versus the placebo group, 19% (7 of 37; P=0.016) and 9% of patients (2 of 23; P=0.038), respectively (FIG. 7A). When evaluated based on prior RBC transfusion burden, RBC-TI ≥8 weeks occurred more frequently in the imetelstat group, 45% of patients (28 of 62) with ≥4 to ≤6 units/8 weeks and 34% of patients (19 of 56) with >6 units/8 weeks, compared with the corresponding frequencies in the placebo group, 21% of patients (7 of 33; P=0.027) and 7% of patients (2 of 27; P=0.023; FIG. 7A). A significantly greater proportion of patients with IPSS low-(40% vs 20.5%; P=0.034) and intermediate-1 risk (40% vs 5%; P=0.004) had 8-week RBC-TI in the imetelstat group compared with the placebo group (FIG. 7A).

Imetelstat treatment met primary end point (8-week TI) with highly statistically significant and clinically meaningful improvement in 8-week TI. These results are shown in Table 8 below:

TABLE 8

| | Imetelstat | Placebo | |
|---|---|---|---|
| | (n = 118) | (n = 60) | P-value* |
| 8-week TI, n (%) | 47 (39.8) | 9 (15.0) | < 0.001 |
| 95% CI | (30.9, 49.3) | (7.1, 26.6) | |

*Cochran Mantel Haenszel test stratified for prior RBC transfusion burden (≤6 units or >6 units of RBCs/8 weeks) and baseline IPSS risk score (Low risk or intermediate-1 risk).
8-week TI = proportion of patients without any RBC transfusion for at least eight consecutive weeks since entry to the trial.

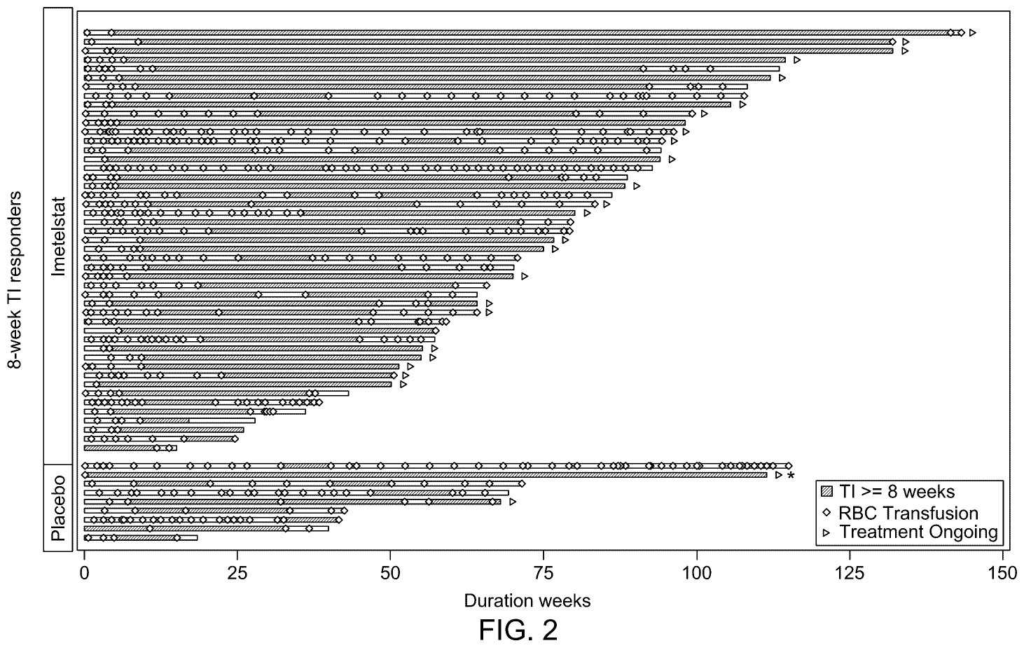

Continuous sustained TI with imetelstat treatment was obtained and 83% of 8-week TI responders had one continuous TI period as shown in FIG. 2.

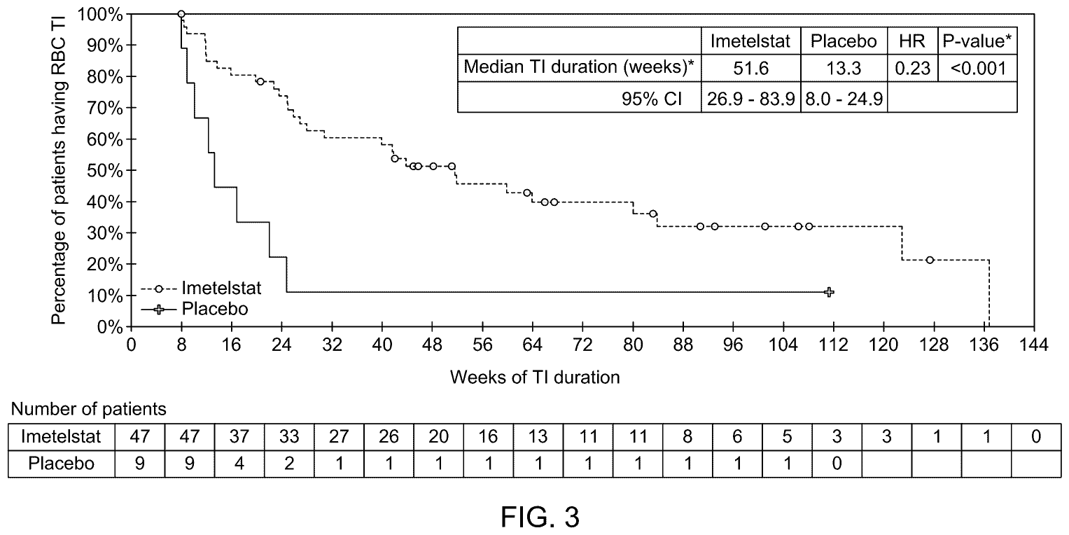

Median duration of TI with imetelstat treatment showed highly statistically significant and clinically meaningful durability of TI upon imetelstat treatment as shown in FIG. 3A.

Imetelstat treatment met key secondary end point, i.e., highly statistically significant and clinically meaningful improvement in 24-week TI was observed as shown in Table 9 below.

TABLE 9

| | Imetelstat | Placebo | |
|---|---|---|---|
| | (n = 118) | (n = 60) | P-value* |
| 24-week TI, n (%) | 33 (28.0) | 2 (3.3) | < 0.001 |
| 95% CI | (20.1, 37.0) | (0.4, 11.5) | |

24-week TI = proportion of patients without any RBC transfusion for at least 24 consecutive weeks since entry to the trial ; CI = confidence interval; NE = non evaluable.

### Key Secondary End Points and Exploratory End Points

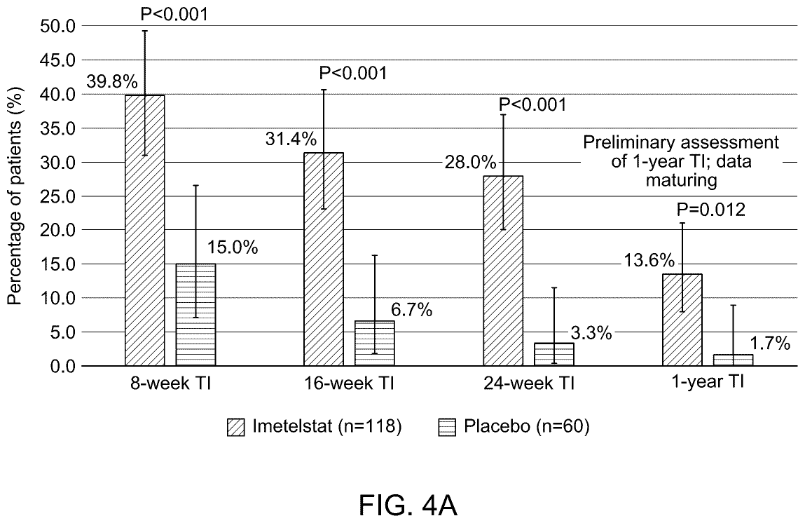

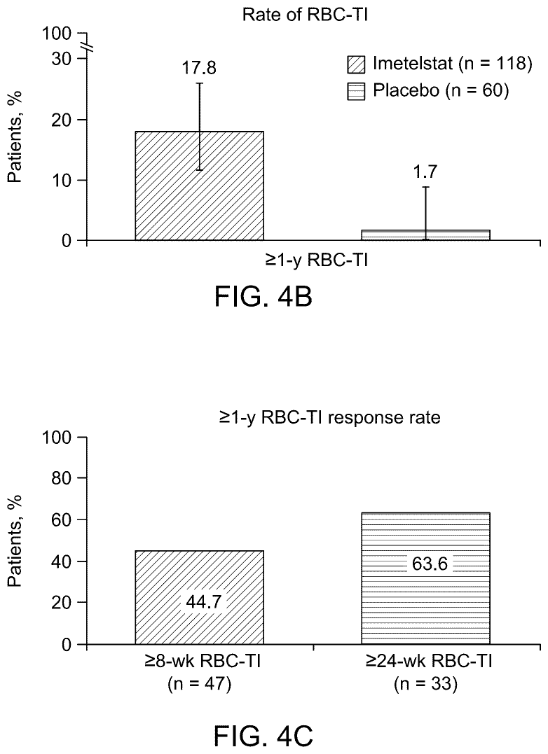

Continuous RBC-TI for at least 24-weeks was demonstrated in 33 patients (28%) in the imetelstat group and 2 patients (3%) in the placebo group (P<0.001; FIG. 4A). Among patients with RS, the rate of 24-week RBC-TI was 33% of patients in the imetelstat group compared with 5% of patients in the placebo group, and the corresponding values were 20% and 0% in patients without RS, respectively. FIG. 7B shows subgroup analysis for 24-week RBC-TI.

Rate of ≥1-year TI in imetelstat treated patients compared to placebo is shown in FIG. 4B and the percentage of patients showing ≥8-week and >24-week TI that also exhibit ≥1-year TI is shown in FIG. 4C. Particularly, 21 of 118 (17.8%) receiving imetelstat achieved ≥1-year TI (95% CI, 11.4-25.9), and 1 of 60 patients (1.7%; 95% CI, 0-8.9) receiving placebo plus supportive care achieved ≥1-year TI. Also, ≥1-year TI was achieved by 44.7% and 63.6% of ≥8-week and >24-week imetelstat-treated responders, respectively.

The correlation between baseline TA, TL, and hTERT levels and clinical benefit showed that imetelstat had higher RBC-TI rates than placebo regardless of TA, TL, and hTERT expression at baseline; however, patients with higher TA, hTERT, or shorter TL were more susceptible to imetelstat treatment, with more significant differences in ≥8-week and >24-week RBC-TI when treated with imetelstat versus placebo (FIGS. 7C-7D), than patients having lower TA and hTERT, or longer TL. These results align with telomere biology in cancer cells and provide evidence for on-target MOA of imetelstat through telomerase inhibition. The results also corroborate the observations from a phase 2 study of imetelstat in patients with myelofibrosis.

With further follow-up, the rate of 1-year RBC-TI was 17.8% of patients (21/118) in the imetelstat group compared with 1.7% of patients (1/60) in the placebo group (P=0.002; FIG. 4).

Among patients meeting the primary end point, the median duration of RBC-TI in the imetelstat group was 51.6 weeks (95% CI, 26.9 to 83.9) compared with 13.3 weeks (95% CI, 8.0 to 24.9) in the placebo group, a 38.3-week difference with hazard ratio (HR) 0.23 (95% CI, 0.091 to 0.571, P<0.001; FIG. 3).

The rates of HI-E according to IWG 2006 and IWG 2018 criteria are shown in Table 10. More patients achieved a ≥1.5 g/dL increase in hemoglobin for ≥8 weeks with imetelstat [140, 34% (95% CI, 25 to 43)] compared with placebo [6, 10% (95% CI, 3.8 to 20.5); P<0.001]. HI-E based on the IWG 2018 criteria 14 was experienced by 50 patients (42%) in the imetelstat group compared with 8 patients (13%) in the placebo group (P<0.001).

TABLE 10
Hematological improvement.

| | Imetelstat | Placebo | % |
|---|---|---|---|
| | (N = 118) | (N = 60) | Difference, |
| End Point | No. of patients (% [95% CI]^{†}) | | P-value* |
| Hematologic improvement | 50 | 8 | 29.0%, |
| (erythroid) - IWG 2018 | (42.4 [33.3, 51.8]) | (13.3 [5.9, 24.6]) | <0.001 |
| LTB^{‡} subjects | 21 | 18 | |
| HI-E response (16-week | 7 | 4 | 11.1% |
| RBC-TI) | (33.3 [14.6, 57.0]) | (22.2 [6.4, 47.6]) | 0.562 |
| HTB^{‡} subjects | 97 | 42 | |
| Major HI-E response | 30 | 0 | 30.9% |
| (16- week RBC-TI) | (21.9, 41.1) | (0.0, 8.4) | <0.001 |
| Minor HI-E response | 43 | 4 | 34.8% |
| (50% RBC units | (34.2, 54.8) | (2.7, 22.6) | <0.001 |
| reduction in 16 weeks) | | | |
| Hematologic improvement | 75 | 31 | 11.9%, |
| (erythroid) - IWG 2006 | (63.6 [54.2, 72.2]) | (51.7 [38.4, 64.8]) | 0.112 |
| Increase in hemoglobin ≥1.5 | 40 | 6 | 23.9% |
| g/dL for ≥8 wk | (33.9 [25.4, 43.2]) | (10.0 [3.8, 20.5]) | <0.001 |
| Transfusion reduction | 71 | 30 | 10.2%, |
| by ≥4 units/8 wk | (60.2 [50.8, 69.1]) | (50.0 [36.8, 63.2]) | 0.175 |

*Cochran-Mantel-Haenszel test, with stratification for prior RBC transfusion burden (≥4 to ≤6 vs. >6 RBC units/8-weeks during a 16-week period prior to randomization) and baseline International Prognostic Scoring System risk category (low vs. intermediate-1) applied to randomization. IWG denotes International Working Group, RBC red blood cell, and wk week.
^{†}Exact Clopper-Pearson confidence interval. Wk denotes week.
^{‡}Per revised IWG 2018, low transfusion burden (LTB) subject is a subject who received 3 to 7 RBC units in the 16 weeks prior to study entry in at least 2 transfusion episodes. High transfusion burden (HTB) subject is a subject who received >8 RBC units in the 16 weeks prior to study entry in at least 2 transfusion episodes.

Among patients achieving 8-week TI, the median increase from pretreatment to peak during longest response-period in blood hemoglobin was 3.55 g/dL in the imetelstat group compared with 0.80 g/dL in the placebo group (FIG. 4A). Over time, patients in the imetelstat group had greater increase in mean hemoglobin levels than those in the placebo group (P<0.001; FIG. 4A). During the trial, patients in the imetelstat group had significantly higher reduction in RBC transfusion units over time compared with those in the placebo group (P=0.042; FIG. 3).

Of the randomized patients, 22% in each group had cytogenetic abnormalities at baseline. Complete or partial cytogenetic responses were observed by an independent review committee in 9 patients (35%) in the imetelstat group and 2 patients (15%) in the placebo group (Table 11). Of 21 imetelstat-treated ≥1-year TI responders, 7 (33%) had cytogenetic abnormality at baseline and >1 posttreatment cytogenetic assessment; of these, 4 (57%) had cCR, 2 (29%) had cPR, and 1 patient (19%) did not meet the response criteria.

TABLE 11
Cytogenetic best response per IWG 2006 criteria

| | | | | |
|---|---|---|---|---|
| | Imetelstat | Placebo | % Difference | |
| N (%) | (N = 118) | (N = 60) | (95% CI) [2] | P value [3] |

| | | | | |
|---|---|---|---|---|
| Patients with baseline | 26 | (22) | 13 | (22) |
| cytogenetic abnormality based | | | | |
| on central lab* | | | | |
| Cytogenetic best response† | | | | |
| Cytogenetic complete | 5 | (19) | 1 | (8) |
| response (CR)^{‡} | | | | |
| Cytogenetic partial | 4 | (15) | 1 | (8) |
| response(PR)^{‡} | | | | |
| Does not meet cytogenetic | 5 | (19) | 5 | (39) |
| CR or PR criteria^{‡} | | | | |
| Not evaluable^{‡} | 12 | (46) | 6 | (46) |
| Cytogenetic CR or PR^{‡} | 9 | (35) | 2 | (15) |

| | | | | |
|---|---|---|---|---|
| 95% CI [1] | (17, 56) | (2, 45) | 19% (−16, 44) | 0.216 |

*Percentages are calculated with the number of patients in each treatment group as the denominator.
†Only patients considered for independent review committees adjudication are those assessed as having baseline cytogenetic abnormality by the IRC based on central lab data.
[1] Exact Clopper-Pearson confidence interval.
[2] The 95% CI is based on Wilson Score method.
[3] The P value is based on Cochran-Mantel-Haenszel (CMH) controlling for prior red blood cell (RBC) transfusion burden (≤6 vs. >6 units RBC) and International Prognostic Scoring System risk group (low vs. intermediate-1) applied to randomization.
^{‡}Percentages are calculated with the number of patients with baseline cytogenetic abnormality based on central lab within each treatment group as the denominator.

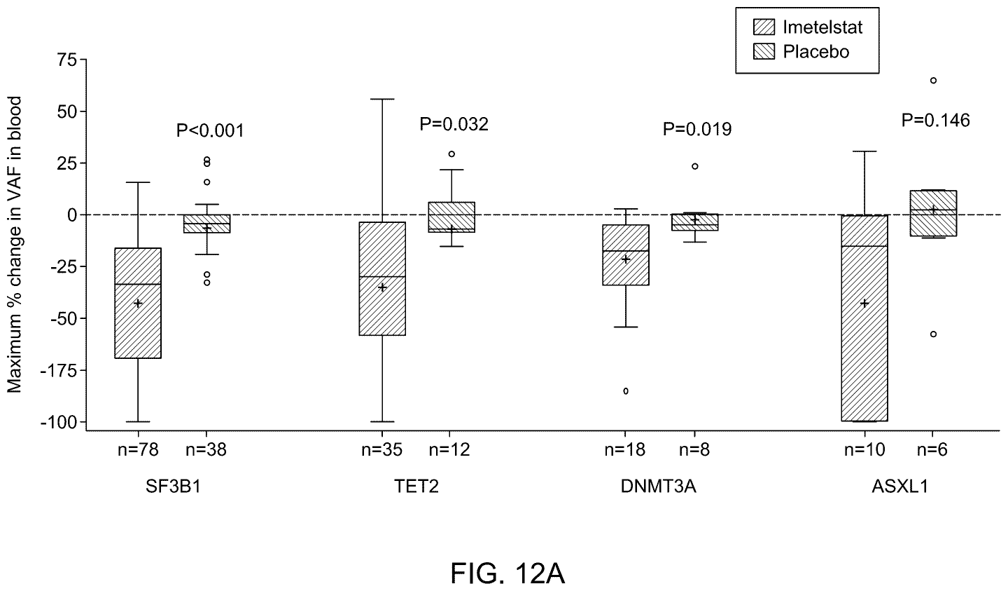

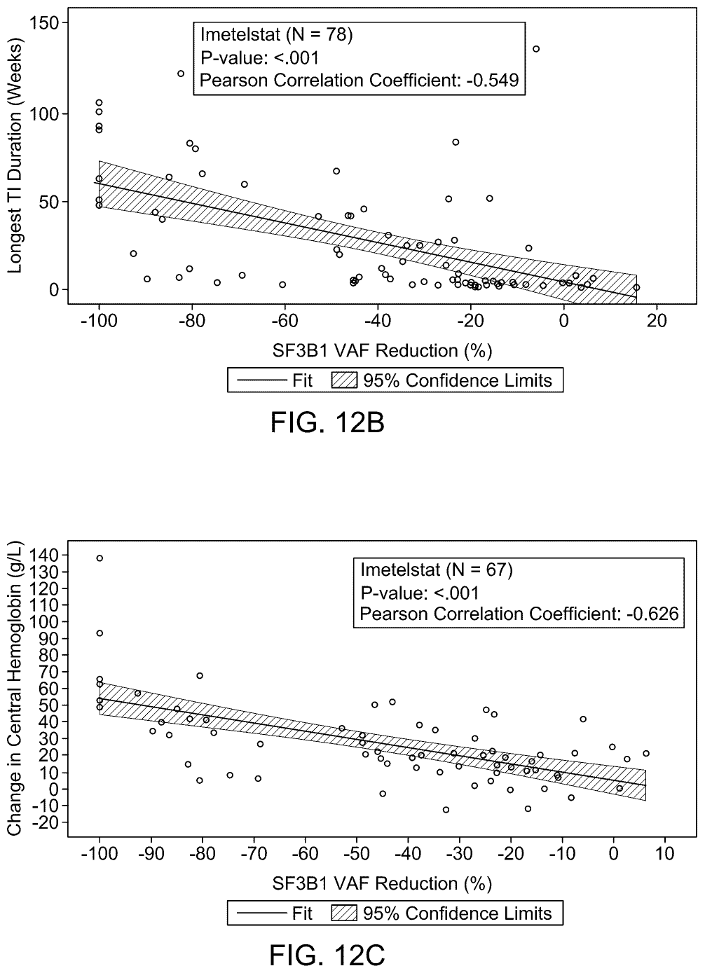

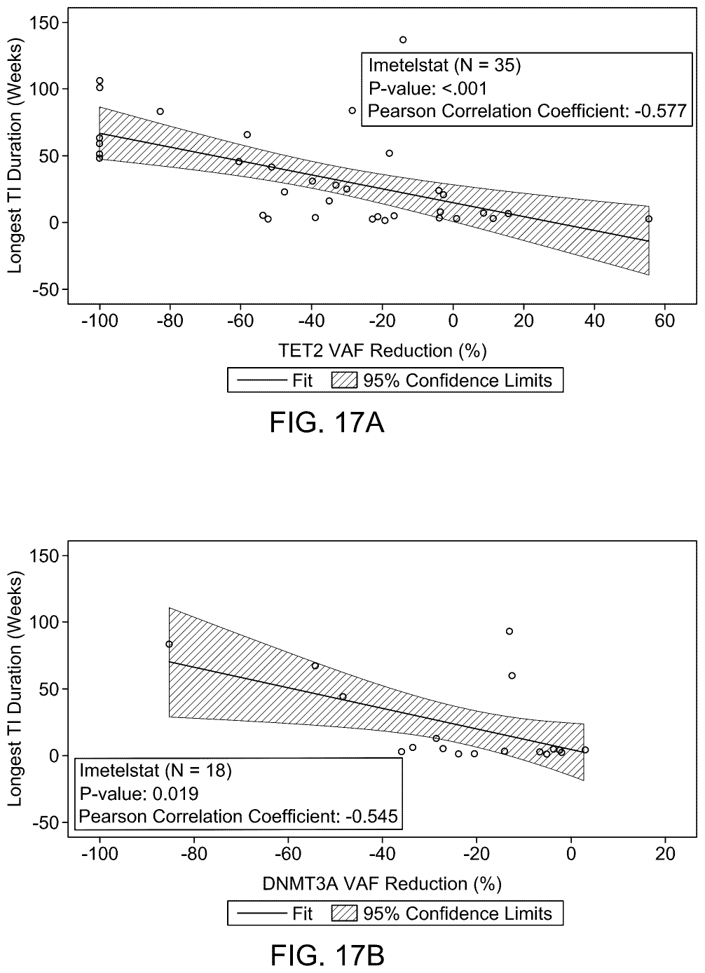

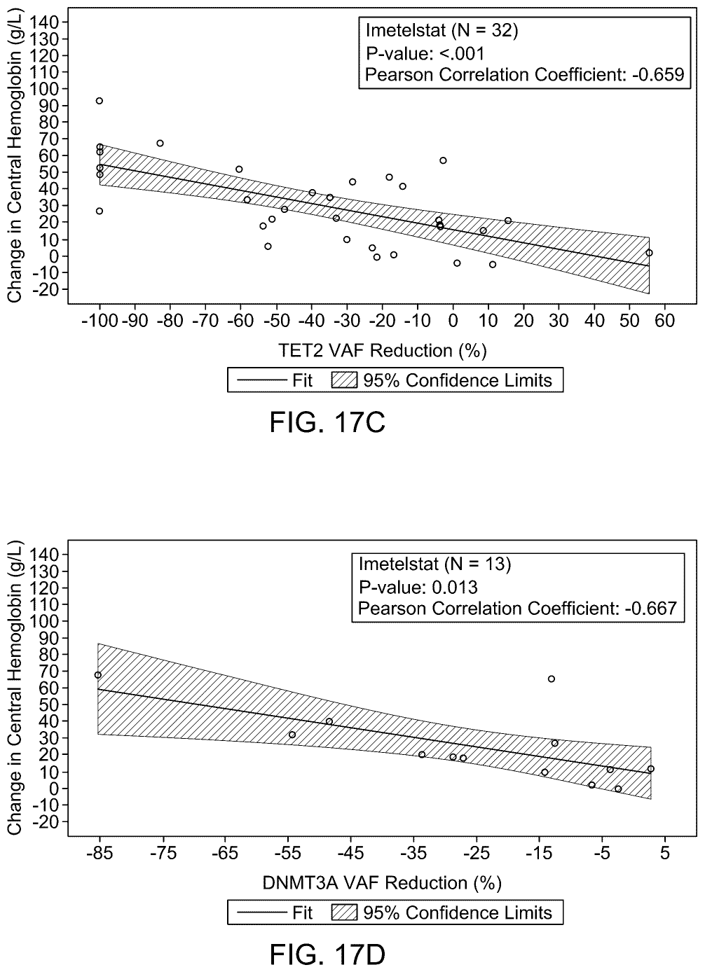

Among patients with evaluable mutation data, the reduction in VAF of SF3B1, TET2, DNMT3A, and ASXL1 genes commonly mutated in MDS were greater with imetelstat than placebo (FIG. 12A) and the reduction in VAF was correlated with increased achievement of 8-week RBC-TI (Table 12), longer RBC-TI duration (FIG. 12B) and increased mean hemoglobin (FIG. 12C) in patients treated with imetelstat. Similar results showed a reduction in TET2 VAF and/or a reduction in DNMT3A VAF correlated with increased achievement of longer RBC-TI duration and increased mean hemoglobin in patients treated with imetelstat. (FIGS. 17A-17D.)

TABLE 12
Response by Participants Who Achieved ≥50%
Max Reduction in Baseline VAF in Imetelstat Arm.

| Patients, n (%) | Imetelstat | | | | | | |
|---|---|---|---|---|---|---|---|
| | SF3B1 VAF ≥50% Reduction | | | | | | |
| | Yes | | No | | Total | | P Value |
| | (N = 23) | | (N = 55) | | (N = 78) | | (Fisher |
| | | | | | | | exact test) |
| 8-Week RBC-TI | | | | | | | |
| Yes | 19 | (82.6) | 21 | (38.2) | 40 | (51.3) | <0.001 |
| No | 4 | (17.4) | 34 | (61.8) | 38 | (48.7) | |
| 24-Week RBC-TI | | | | | | | |
| Yes | 16 | (69.6) | 13 | (23.6) | 29 | (37.2) | <0.001 |
| No | 7 | (30.4) | 42 | (76.4) | 49 | (62.8) | |
| 1-Year RBC-TI | | | | | | | |
| Yes | 11 | (47.8) | 3 | (5.5) | 14 | (17.9) | <0.001 |
| No | 12 | (52.2) | 52 | (94.5) | 64 | (82.1) | |
| | Imetelstat | | | | | | |
| Patients, n (%) | TET2 VAF ≥50% Reduction | | | | | | |
| | | | | | | | P Value |
| | Yes | | No | | Total | | (Fisher |
| | (N=12) | | (N=23) | | (N=35) | | exact test) |
| 8-Week RBC-TI | | | | | | | |
| Yes | 10 | (83.3) | 10 | (43.5) | 20 | (57.1) | 0.034 |
| No | 2 | (16.7) | 13 | (56.5) | 15 | (42.9) | |
| 24-Week RBC-TI | | | | | | | |
| Yes | 10 | (83.3) | 6 | (26.1) | 16 | (45.7) | 0.003 |
| No | 2 | (16.7) | 17 | (73.9) | 19 | (54.3) | |
| 1-Year RBC-TI | | | | | | | |
| Yes | 6 | (50.0) | 2 | (8.7) | 8 | (22.9) | 0.011 |
| No | 6 | (50.0) | 21 | (91.3) | 27 | (77.1) | |

The analysis represents patients in imetelstat arm in biomarker analysis set who had detectable assessment (≥5% VAF) in SF3B1 or TET2 at baseline and had at least one postbaseline mutation assessment.
Percentages are calculated with respect to each column.

P value was determined using Cochran Mantel Haenszel test stratified for prior RBC transfusion burden (≤6 units or >6 units of RBCs/8 weeks) and baseline IPSS risk score (Low or Intermediate-1 risk). Median TI duration (Kaplan-Meier estimates of duration of RBC TI) for imetelstat patients who achieved 24-week TI was 80.0 weeks (51.6, NE). Approximately 70% of patients treated with imetelstat who achieved 8-week TI, went on to achieve 24-week TI.

TI rates were assessed over long periods and increasing magnitude of benefit was observed imetelstat compared placebo with longer TIs as shown in FIG. 4.

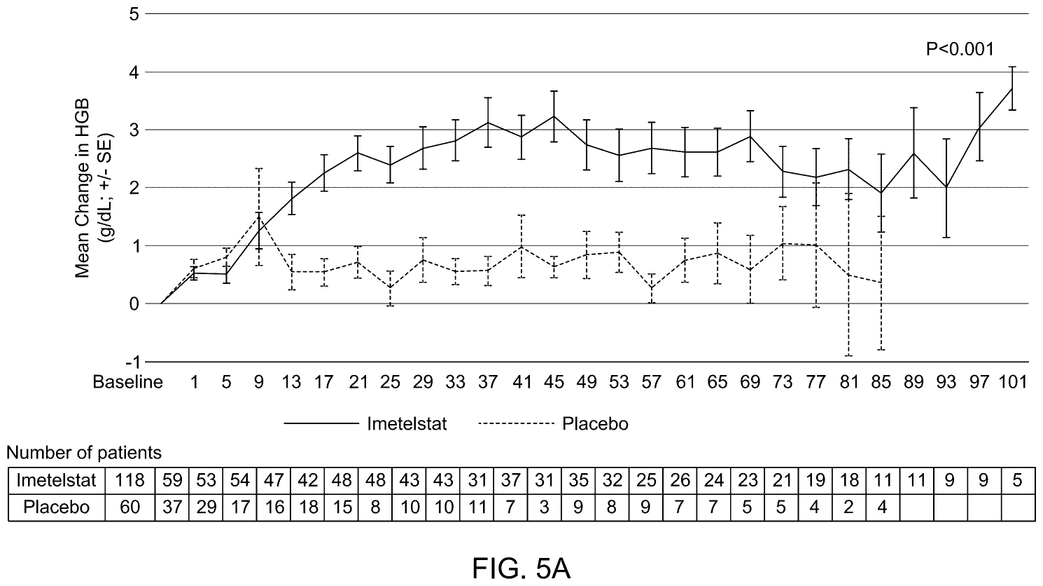

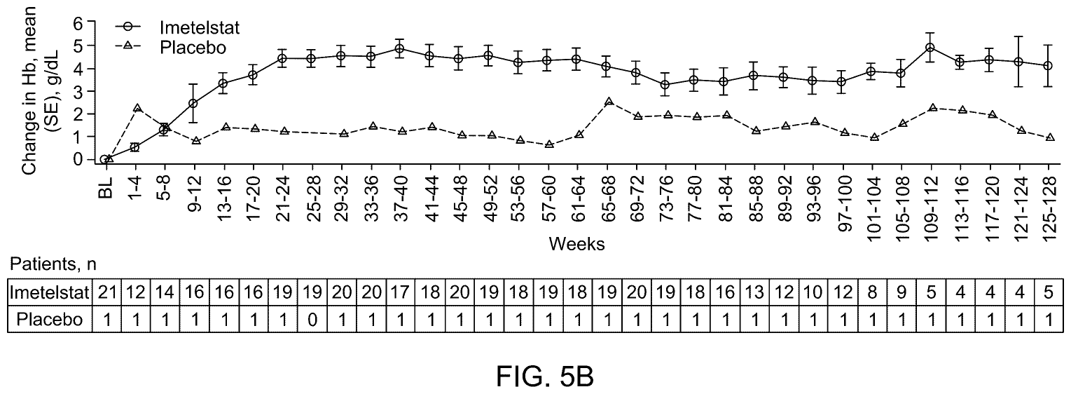

Highly statistically significant increase in hemoglobin levels was observed in imetelstat treated patients as compared to placebo as shown in FIG. 5. For 8-week TI responders median hemoglobin increase for imetelstat was 3.6 g/dL as compared to 0.8 g/dL for placebo. Also, for 8-week TI responders, median hemoglobin peak was 11.3 g/dL as compared to 8.9 g/dL for placebo. This analysis was performed during TI period.

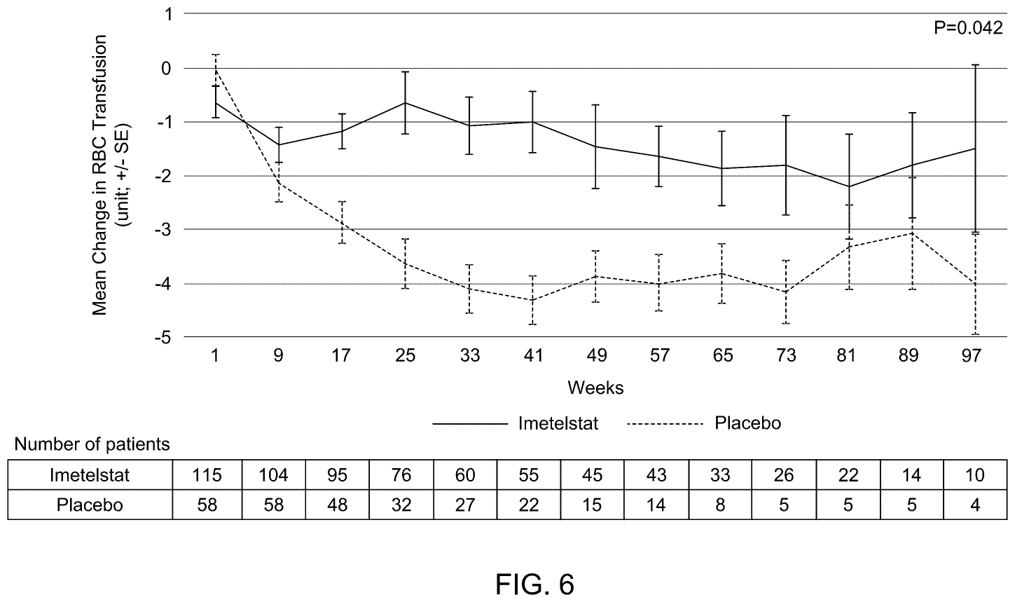

Statistically significant decrease in the number of RBC units transfused was observed in imetelstat treated patients as compared to placebo as shown in FIG. 6.

Another secondary end point, particularly, hematologic improvement-erythroid (HI-E) was analyzed. Highly statistically significant HI-E was observed using updated IWG 2018 criteria. Evolution of more recent HI-E criteria (IWG-2018) puts greater emphasis on durability by measuring response at >16 weeks, although HI-E using protocol-specified IWG 2006 criteria was not statistically significant.

TABLE 13

| | Imetelstat | Placebo | |
|---|---|---|---|
| | (n = 118) | (n = 60) | P-value * |
| HI-E per IWG 2018 , n (%) | 50 (42.4) | 8 (13.3) | <0.001 |
| 95% CI, % | (33.3, 51.8) | (5.9, 24.6) | |
| 16-week TI, n (%) | 37 (31.4) | 4 (6.7) | <0.001 |
| 95% CI, % | (23.1, 40.5) | (1.9, 16.2) | |
| Transfusion reduction by | 51 (43.2) | 9 (15.0) | <0.001 |
| 50%/16 weeks | | | |
| 95% CI, % | (34.1, 52.7) | (7.10, 26.6) | |
| HI-E per IWG 2006, n (%) | 75 (63.6) | 31 (51.7) | 0.112 |
| 95% CI, % | (54.2, 72.2) | (38.4, 64.8) | |
| ≥1.5 g/dL increase in Hgb ≥8 | 40 (33.9) | 6 (10.0) | <0.001 |
| weeks, n (%) | | | |
| 95% CI, % | (25.4, 43.2) | (3.8, 20.5) | |
| Transfusion reduction by ≥4U/8 | 71 (60.2) | 30 (50.0) | 0.175 |
| weeks, n (%) | | | |
| 95% CI, % | (50.8, 69.1) | (36.8, 63.2) | |

*Cochran Mantel Haenszel test stratified for prior RBC transfusion burden (≤6 units or >6 units of RBCs/8 weeks) and baseline IPSS risk score (Low risk or intermediate-1 risk).
CI = confidence interval; Hgb = hemoglobin; HI-E= hematologic improvement-erythroid; IWG = International Working Group.

Statistically significant 8-week TI rates (p<0.05) and comparable magnitude of clinical benefit demonstrated across all subgroups as shown in FIG. 7A.

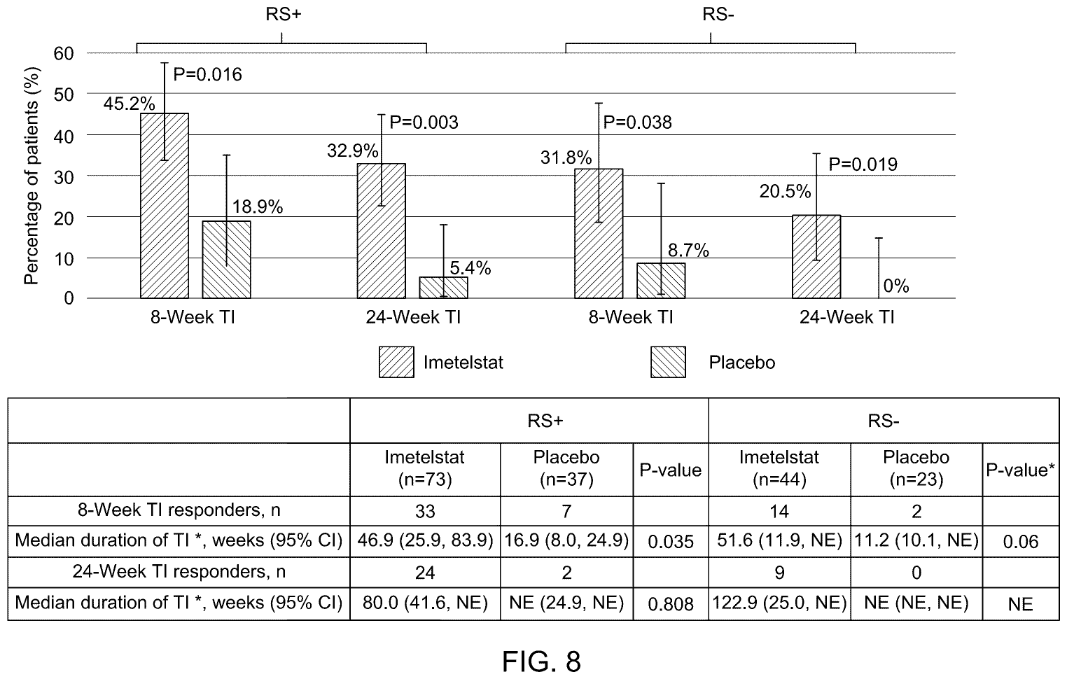

RS+ and RS− groups demonstrate statistically significant improvement in both 8-week and 24-week TI as shown in FIG. 8.

The primary end point was met as shown in Table 14. 39.8% vs 15.0% of patients receiving imetelstat or placebo, respectively, achieved 8-wk TI, p<0.001. The rate of 8-wk TI was statistically significantly higher with imetelstat vs placebo: 83% of 8-week RBC-TI responders had a single continuous RBC-TI period (FIG. 15). Median TI duration was significantly longer for imetelstat vs placebo as shown in Table 14. 51.6 vs 13.3 wks, p<0.001. Patients receiving imetelstat had significantly higher mean hemoglobin (p<0.001) and fewer transfusions (p=0.042) over time than those on placebo.

The primary end point was achieved in patients with and without RS, 45% (33 of 73) and 32% of patients (14 of 44) in the imetelstat group, versus the placebo group, 19% (7 of 37; P=0.016) and 9% of patients (2 of 23; P=0.038), respectively (FIG. 7A). When evaluated based on prior RBC transfusion burden, RBC-TI ≥8 weeks occurred more frequently in the imetelstat group, 45% of patients (28 of 62) with ≥4 to ≤6 units/8 weeks and 34% of patients (19 of 56) with >6 units/8 weeks, compared with the corresponding frequencies in the placebo group, 21% of patients (7 of 33; P=0.027) and 7% of patients (2 of 27; P=0.023; FIG. 7A). A significantly greater proportion of patients with IPSS low-(40% vs 20.5%; P=0.034) and intermediate-1 risk (40% vs 5%; P=0.004) had 8-week RBC-TI in the imetelstat group compared with the placebo group (FIG. 7A).

TABLE 14
Phase 3 Clinical end points

| | Imetelstat | | Placebo | | |
|---|---|---|---|---|---|
| Measure, n (%) | (N = 118) | | (N = 60) | | P^{a} |
| 8-wk TI | 47 | (39.8) | 9 | (15.0) | <0.001 |
| TI duration, median | 51.6 | (26.9-83.9) | 13.3 | (8.0-24.9) | <0.001^{d} |
| wks (95% CI)^{b} | | | | | |
| 24-wk TI | 33 | (28.0) | 2 | (3.3) | <0.001 |
| HI-E^{c} | 50 | (42.4) | 8 | (13.3) | <0.001 |

^{a}Cochran Mantel Haenszel test stratified by prior transfusion burden and IPSS risk;
^{b}8-wk TI, Kaplan-Meier estimate;
^{c}2018 IWG;
^{d}Stratified log-rank test

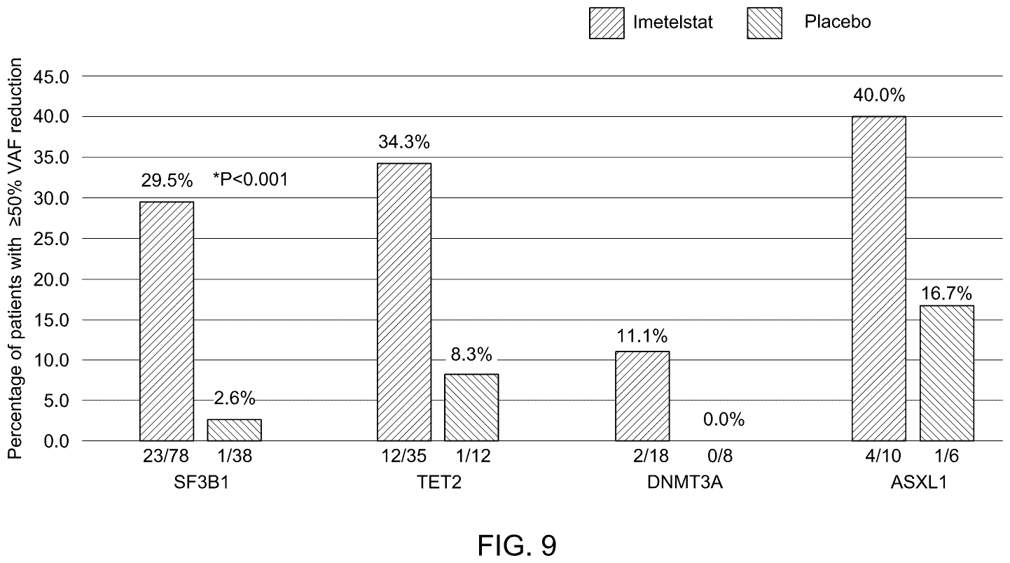

FIG. 9 shows that patients that responded to imetelstat therapy showed 50% or more reduction in VAF for the following genes: SF3B1, TET2, DNMT3A and ASXL1. These data show that 50% or more reduction in VAF for one or more of the following genes: SF3B1, TET2, DNMT3A and ASXL1 could be used to identify and specifically treat patients with MDS because such patients have an increased likelihood of benefiting from treatment with a telomerase inhibitor.

In 3 of 4 genes frequently mutated in MDS, VAF reduction was significantly greater in patients treated with imetelstat than those receiving placebo: SF3B1 (p<0.001), TET2 (p=0.032), DNMT3A (p=0.019) and ASXL1 (p=NS). SF3B1 VAF reduction correlated with longer TI duration (r=0.549, p<0.001).

No new safety signals were identified. The most common Grade 3/4 adverse events were thrombocytopenia and neutropenia, with similar rates of Grade≥3 bleeding and infections on imetelstat and placebo. In imetelstat treated patients, cytopenias were manageable. They were of short duration and reversible to Grade≤2 within 4 weeks in more than 80% of patients.

Grade 3-4 thrombocytopenia and neutropenia occurred in 14 (67%) and 20 (95%) patients with TI ≥1 year. For grade 3/4 neutropenia and thrombocytopenia events, the mean (SD) duration was 1.78 (1.58) and 2.25 (2.48) weeks, respectively. 81% of grade 3/4 neutropenia and 89% of grade 3/4 thrombocytopenia were reversible to grade≤2 within 4 weeks.

These data also show that imetelstat treatment for MDS exhibits depth, breadth, and durability of TI. Highly statistically significant (p<0.001) and clinically meaningful improvements were observed in imetelstat treated patients in 8-week TI, 16-week TI and 24-week TI versus placebo. Median TI duration for imetelstat-treated patients approached one year.

Highly statistically significant (p<0.001) and clinically meaningful increases was observed in mean change in hemoglobin levels over time for imetelstat-treated patients versus placebo.

HI-E per IWG 2018 criteria demonstrated a highly statistically significant (p<0.001) and clinically meaningful improvement for imetelstat-treated patients as compared to placebo.

Statistically significant (p<0.05) and clinically meaningful 8-week TI rate achieved both for imetelstat-treated patients with high and very high transfusion burdens; low and intermediate-1 IPSS risk; and RS+ and RS− versus placebo.

Clinical and molecular evidence that supports MDS disease modification potential with imetelstat include: one-year median TI duration for imetelstat 8-week TI responders, a median rise of 3.6 g/dL in hemoglobin levels in imetelstat 8-week TI responders, and >50% VAF decreases in SF3B1, TET2, DNMT3A and ASXL1 mutations.

Thus, Imetelstat treatment demonstrated clinically meaningful 8- and 24-week TI rates and prolonged TI duration, increased hemoglobin, and reduction in VAF in heavily RBC TD ESA R/R non-del(5q) lower-risk myelodysplastic syndrome (LR-MDS) patients naive to len/HMAs.

A main therapeutic goal in LR-MDS patients is to alter disease biology by eradicating malignant clones, but not be at the expense of toxicity. MDS-initiating cells carrying cytogenetic abnormalities, mutant alleles, or both arise from malignant stem and progenitor cells. SF3B1, involved in RNA splicing, and TET2, involved in DNA methylation, are recurrently mutated genes in LR-MDS, which can be quantified by measuring change in VAF to denote disease burden. In a Phase 3 trial, treatment with imetelstat demonstrated statistically significant and clinically meaningful efficacy over placebo with the over-8-week TI rate of 40%.

As noted above, patients with heavily RBC-TD-ESA relapsed/refractory or ineligible non-del(5q) LR-MDS naive to lenalidomide and hypomethylating agents (len/HMA) received 2-hr infusion of imetelstat 7.5 mg/kg or placebo every 4 weeks. An independent review committee assessed abnormal cytogenetic profiles of bone marrow samples taken pre- and every 24 weeks post-treatment to assess cytogenetic response of complete or partial remission. Of the 178 randomized patients, 22.0% in the imetelstat group and 21.7% in the placebo group had baseline cytogenetic abnormalities and post-treatment samples. Cytogenetic response was observed in 9/26 patients (34.6%, 95% CI=17.2-55.7) in the imetelstat group and 2/13 patients (15.4%, 95% CI=1.9-45.5) in the placebo group.

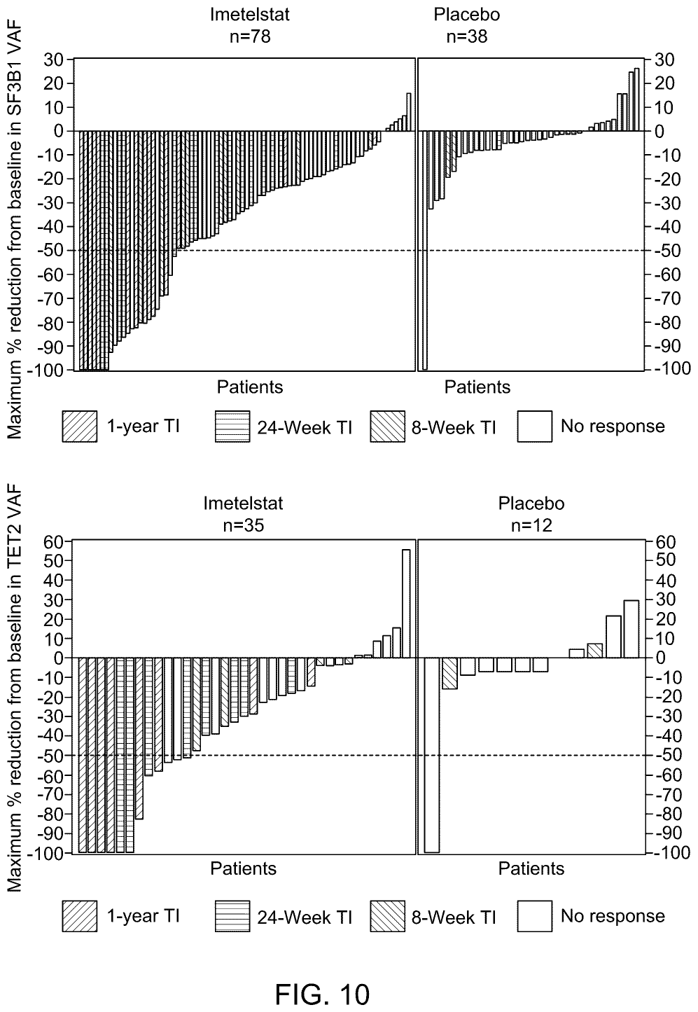

VAF changes were assessed from blood taken pre- and every 12 weeks post-treatment in patients with greater than or equal to 5% variant allele at baseline and >1 post-baseline assessment. T-tests evaluated group comparison, Fisher's exact test evaluated association, and linear regression evaluated correlation. Imetelstat-treated patients demonstrated a higher rate of ≥50% VAF decreases in SF3B1, TET2, DNMT3A, and ASXL1 mutations as compared with placebo (FIG. 9). The patients achieving ≥8-weeks, ≥24-weeks, and >1-year TI in the imetelstat group were enriched with patients who had ≥50% reductions in SF3B1 and TET2 VAF compared with placebo (FIG. 10). Additionally, both 8- and 24-week TI responders in the imetelstat arm had significantly greater reductions in SF3B1 VAF or in TET2 VAF vs non responders (p<0.001, for all cases). Furthermore, there were significantly higher rates of 8-week (82.6% vs 38.2%), 24-week (69.6% vs. 23.6%) and 1-year (47.8% vs. 5.5%) TI responders in patients who had ≥50% VAF in SF3B1 compared to subjects who didn't achieve ≥50% reduction (p<0.001, in all cases). Importantly, greater reductions in SF3B1 VAF in the imetelstat arm correlated significantly with hemoglobin increase; r=−0.626, p<0.001, and longer TI duration; r=−0.549, p<0.001 (FIGS. 12A-12C).

Thus, in the phase 3 imetelstat study, more heavily RBC TD ESA R/R/ineligible non-del(5q) LR-MDS patients naive to len/HMA treated with imetelstat experienced cytogenetic response and reduction in SF3B1, TET2, DNMT3A, and ASXL1 mutational burden as compared to placebo. Furthermore, SF3B1 VAF reduction correlated with clinically meaningful end points of increased hemoglobin and TI duration. Taken together with robust rates of TI that are continuous and durable may indicate improvement of the ineffective erythropoiesis characteristic of LR-MDS and suggests the potential of imetelstat to modify the biology of disease in these patients.

As noted above, Phase 2 of the imetelstat study, demonstrated that treatment with imetelstat resulted in prolonged, durable TI across a broad range of heavily RBC TD ESA relapsed/refractory non-del(5q) LR-MDS patients naive to lenalidomide and hypomethylating agents (len/HMA).

As mentioned above, the primary end point was 8-week TI rate; subgroup analyses included IPSS risk, prior transfusion burden, and RS status. Secondary end points included 24-week TI rate, TI duration, and hematologic improvement-erythroid (HI-E) rate. Changes in VAF was exploratory. The primary and key secondary end points were compared using a Cochran Mantel Haenszel test stratified by prior transfusion burden and IPSS category. TI duration was calculated by Kaplan-Meier method and compared by stratified log-rank test.

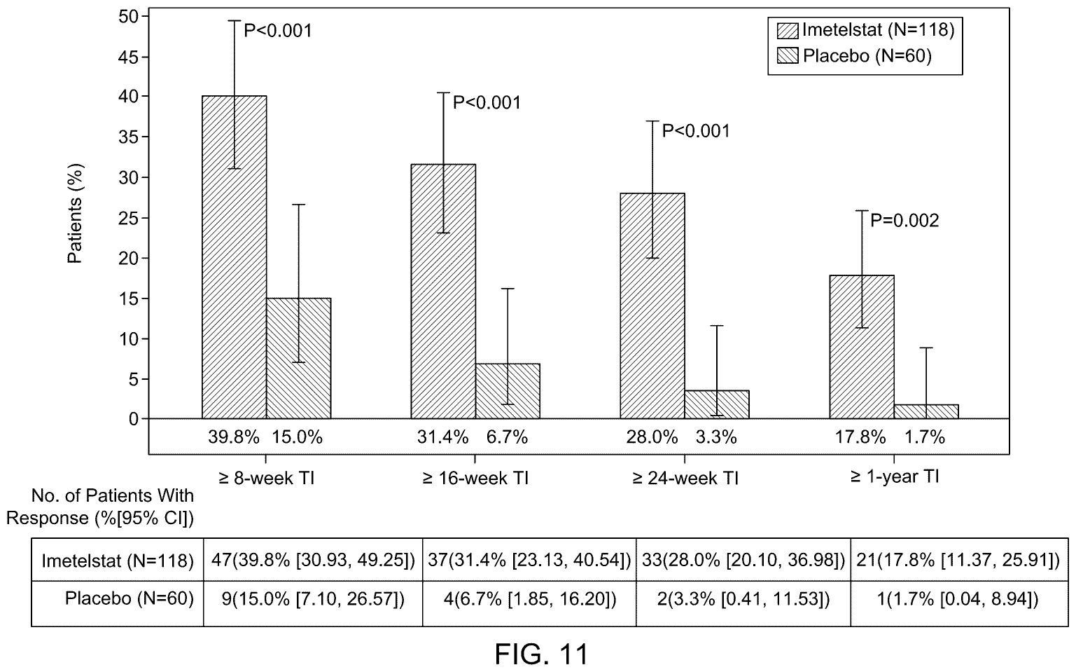

Imetelstat treatment met the primary end point; 47 patients (39.8%) vs 9 patients (15.0%) receiving imetelstat vs placebo achieved 8-week TI, p<0.001. The rate of 8-week TI was also significantly higher with imetelstat vs placebo across subgroups, including in RS negative patients. Median TI duration (95% CI) was 51.6 (26.9-83.9) weeks with imetelstat vs 13.3 (8.0-24.9) weeks with placebo, p<0.001. Twenty-four-week TI was achieved in 33 patients (28.0%) receiving imetelstat versus 2 patients (3.3%) receiving placebo, p<0.001. With additional 3-month follow-up, 21 patients (17.8%) on imetelstat versus 1 patient (1.7%) on placebo achieved ≥1-yr TI, p=0.002, representing 63.6% of ≥24-week imetelstat TI responders (FIG. 11). HI-E rates (2018 IWG, emphasizing >16-wk response) were 42.4% with imetelstat vs 13.3% with placebo, p<0.001. Patients receiving imetelstat had significantly higher mean hemoglobin (p<0.001) and fewer transfusions (p=0.042) over time than those on placebo. VAF reduction in 3 genes frequently mutated in MDS was significantly greater in patients treated with imetelstat than placebo: SF3B1 (p<0.001), TET2 (p=0.032), DNMT3A (p=0.019) and ASXL1 (p=NS). SF3B1 VAF reduction correlated with hemoglobin increase (p<0.001) and longer TI duration in imetelstat-treated patients, p<0.001.

Thus, imetelstat demonstrated statistically significant and clinically meaningful efficacy with robust 8-week, 24-week, and 1-year TI rates and prolonged continuous TI duration. For this LR-MDS patient population, almost one fifth of imetelstat treated patients achieved continuous TI for ≥1 year, representing substantial relief from transfusion-associated complications. VAF reduction and its correlation to clinical end points, including durable TI, support imetelstat's disease-modifying potential. Safety results were consistent with prior reports. Imetelstat treatment provides significant clinical benefit to a heavily TD LR MDS patient population in need of novel therapy.

### Safety

The most frequently reported AEs were neutropenia and thrombocytopenia. AEs of any grade and occurring in 10% of patients in either study group are shown in Table 15 with most nonhematological adverse events (AEs) occurring at low grades.

TABLE 15
Treatment-emergent adverse events occurring in at
least 10% of patients in the safety population .*

| | Imetelstat (N = 118) | | | | Placebo (N = 59) | | | |
|---|---|---|---|---|---|---|---|---|
| | Any Grade | | Grade 3-4 | | Any Grade | | Grade 3-4 | |
| Event | No. of patients (percent) | | | | | | | |
| Hematologic | | | | | | | | |
| Thrombocytopenia | 89 | (75) | 73 | (62) | 6 | (10) | 5 | (8) |
| Neutropenia | 87 | (74) | 80 | (68) | 4 | (7) | 2 | (3) |
| Anemia | 24 | (20) | 23 | (19) | 6 | (10) | 4 | (7) |
| Leukopenia | 12 | (10) | 9 | (8) | 1 | (2) | 0 | |
| General disorders | | | | | | | | |
| and administration | | | | | | | | |
| site conditions | | | | | | | | |
| Asthenia | 22 | (19) | 0 | | 8 | (14) | 0 | |
| Edema peripheral | 13 | (11) | 0 | | 8 | (14) | 0 | |
| Pyrexia | 9 | (8) | 2 | (2) | 7 | (12) | 0 | |
| Infections and | | | | | | | | |
| infestations | | | | | | | | |
| COVID-19 | 22 | (19) | 3 | (3)* | 8 | (14)† | 3 | (5)‡ |
| Gastrointestinal | | | | | | | | |
| disorders | | | | | | | | |
| Diarrhea | 14 | (12) | 1 | (1) | 7 | (12) | 1 | (2) |
| Constipation | 9 | (8) | 0 | | 7 | (12) | 0 | |
| Nervous system | | | | | | | | |
| disorders | | | | | | | | |
| Headache | 15 | (13) | 1 | (1) | 3 | (5) | 0 | |
| Investigations | | | | | | | | |
| ALT increased | 14 | (12) | 3 | (3) | 4 | (7) | 2 | (3) |
| Hepatobiliary | | | | | | | | |
| disorders | | | | | | | | |
| Hyperbilirubinemia | 11 | (9) | 1 | (1) | 6 | (10) | 1 | (2) |

ALT denotes alanine aminotransferase.
†Includes COVID-19, asymptomatic COVID-19, and COVID-19 pneumonia.
‡Only COVID-19 pneumonia events were Grade 3-4 COVID-19.

Overall, 107 patients (91%) receiving imetelstat and 28 patients (48%) receiving placebo had grade 3 or 4 AEs (Table 16). A total of 38 patients (32%) receiving imetelstat had at least one serious AE compared with 13 patients (22%) receiving placebo (Table 16).

TABLE 16
Summary of TEAEs and serious TEAEs.

| | Imetelstat | | Placebo | |
|---|---|---|---|---|
| | (N = 118) | | (N = 59) | |
| Summary of TEAEs - No. (%) | | | | |
| Patients with at least 1 TEAE | 117 | (99.2) | 59 | (100.0) |
| Patients experiencing at least 1 serious | 38 | (32.2) | 13 | (22.0) |
| TEAE | | | | |
| Patients with at least 1 suspected related | 85 | (72.0) | 6 | (10.2) |
| Grade ≥3 TEAE | | | | |
| Patients with at least 1 Grade 3-4 TEAE | 107 | (90.7) | 28 | (47.5) |
| Serious TEAEs by preferred term | | | | |
| (>3% incidence) - No. (%) | | | | |
| COVID-19 pneumonia | 2 | (1.7) | 3 | (5.1) |

| | | | |
|---|---|---|---|
| Abscess limb | 0 | 2 | (3.4) |

Treatment-emergent adverse events (TLALs) include events that 1) occur after the first dose of study drug, through the treatment phase, and for 30 days following the last dose of study drug or until subsequent anticancer therapy if earlier; 2) any event that is considered study drug-related regardless of the start date of the event; or 3) any event that is present at baseline but worsens in severity or is subsequently considered drug-related by the investigator.
Adverse events were coded using Medical Dictionary for Regulatory Activities Version 25.0.

The most common grade 3-4 treatment-emergent hematologic ALs in the imetelstat group were neutropenia and thrombocytopenia in 80 (68%) and 73 patients (62%), respectively. Median duration of neutropenia was 1.9 weeks; 81% of grade 3-4 events resolved to grade≤2 within 4 weeks. The median duration of thrombocytopenia was 1.4 weeks; and 86% of grade 3-4 events resolved to grade≤2 within 4 weeks. (Table 17).

TABLE 17
Duration and Reversibility of Worsening Grade 3 or 4 Neutropenia
and Thrombocytopenia Events by Lab Assessment During the Study.

| | Imetelstat | | Placebo | |
|---|---|---|---|---|
| | (N = 118) | | (N = 59) | |
| G3/4 Neutropenia events - | 279 | | 6 | |
| no. of events | | | | |
| Median duration - Weeks (range) | 1.86 | (0-15.9) | 2.21 | (1.0-4.6) |
| Resolved >4 weeks* - | 40 | (14.3) | 2 | (33.3) |
| No. of Events (%) | | | | |
| Resolved <4 weeks* - | 226 | (81.0) | 3 | (50.0) |
| No. of Events (%) | | | | |
| Ongoing † | 13 | (4.7) | 1 | (16.7) |
| G3/4 Thrombocytopenia events - | 212 | | 9 | |
| no. of events | | | | |
| Median duration - Weeks (range) | 1.43 | (0.1-12.6) | 2.00 | (0.3-11.6) |
| Resolved ≥4 weeks* - | 17 | (8.0) | 1 | (11.1) |
| No. of Events (%) | | | | |
| Resolved <4 weeks* - | 183 | (86.3) | 4 | (44.4) |
| No. of Events (%) | | | | |
| On-going † - | 12 | (5.7) | 4 | (44.4) |
| No. of Events (%) | | | | |

*Grade 3/4 cytopenia resolved to ≤ Grade 2.
† Ongoing may include events on-going at time of start of subsequent anticancer therapy if any, study discontinuation or death, or ongoing at the time of study cutoff.
A patient can report multiple events for each lab assessment. G denotes Grade.

A majority of patients experiencing these events did so within the first 3 treatment cycles (Table 18).

TABLE 18
Maximum Postbaseline CTCAE Grade, Worsened Since Baseline
for Thrombocytopenia and Neutropenia by Cycle.

| | Neutropenia | | Thrombocytopenia | |
|---|---|---|---|---|
| | Cycle 1-3 | | | |
| Grade - No. | Imetelstat | Placebo | Imetelstat | Placebo |
| of Patients (%) | (N = 118) | (N = 59) | (N = 118) | (N = 59) |
| 3 | 52 (44.1) | 2 (3.4) | 36 (30.5) | 1 (1.7) |
| 4 | 26 (22.0) | 0 | 21 (17.8) | 0 |
| Grade - No. | Cycle 4-6 | | | |
| of Patients (%) | Imetelstat | Placebo | Imetelstat | Placebo |
| | (N = 103) | (N = 53) | (N = 103) | (N = 53) |
| 3 | 31 (30.1) | 1 (1.9) | 27 (26.2) | 2 (3.8) |
| 4 | 5 (4.9) | 1 (1.9) | 5 (4.9) | 0 |
| Grade - No. | Cycle 7-9 | | | |
| of Patients (%) | Imetelstat | Placebo | Imetelstat | Placebo |
| | (N = 76 | (N = 41) | (N = 76) | (N = 41) |
| 3 | 17 (22.4) | 0 | 19 (25.0) | 1 (2.4) |
| 4 | 3 (3.9) | 0 | 2 (2.6) | 0 |
| Grade - No. | Cycle 10-12 | | | |
| of Patients (%) | Imetelstat | Placebo | Imetelstat | Placebo |
| | (N = 53 | (N = 25) | (N = 53) | (N = 25) |
| 3 | 10 (18.9) | 1 (4.0) | 9 (17.0) | 0 |
| 4 | 1 (1.9) | 0 | 0 | 1 (4.0) |
| Grade - No. | Cycle ≥13 | | | |
| of Patients (%) | Imetelstat | Placebo | Imetelstat | Placebo |
| | (N = 48 | (N 19) | (N = 48) | (N = 19) |
| 3 | 15 (31.3) | 1 (5.3) | 11 (22.9) | 0 |
| 4 | 1 (2.1) | 0 | 1 (2.1) | 0 |

Worsened was defined as CTCAE grade elevated after baseline. The grade 1-4 summaries categorize patients according to the maximum grade lab among those labs that have worsened since baseline.
Percentage is calculated from the patients in each group with a nonmissing baseline and postbaseline parameter value as the denominator. CTCAE denotes Common Terminology Criteria for Adverse Events.

Most of the dose reductions and delays in the imetelstat group were due to neutropenia (33.1% and 50.8%, respectively) and/or thrombocytopenia (22.9% and 46.6%, respectively). Clinical consequences from cytopenias of grade 3-4 bleeding events, infections, and febrile neutropenia were similar in patients treated with imetelstat and placebo (Table 19).

TABLE 19
Clinical consequences of cytopenias.

| Event, n (%) | Imetelstat (N = 118) | Placebo (N = 59) |
|---|---|---|
| Grade ≥3 bleeding | 3 (3) | 1 (2) |
| events* | | |
| Grade ≥3 infections† | 13 (11) | 8 (14) |
| Grade 3 febrile‡ | 1 (1) | 0 |
| neutropenia | | |

*No ≥Grade 3 bleeding events in the setting of Grade 3/4 thrombocytopenia; on imetelstat: 2 patients with Grade 4 gastrointestinal bleeding, unrelated and resolved and 1 Grade 3 hematuria, unrelated and resolved.
†On imetelstat: 3 patients with Grade 3/4 infections in setting of Grade 3/4 neutropenia; all 3 were sepsis and resolved with only 1 considered related.
‡Occurred at day 33, lasted 8 days; assessed by investigator as possibly related to imetelstat; patient subsequently achieved TI >40 weeks and remains on treatment at data cutoff.

Table 20 below provides certain hepatic events observed in the study subjects.

TABLE 20
TEAEs of interest: hepatic events

| | Event | | | |
|---|---|---|---|---|
| | Imetelstat (N = 118) | | Placebo (N = 59) | |
| Liver function | Any Grade | Grade 3-4 | Any Grade | Grade 3-4 |
| test abnormality | No. of patients (percent) | | | |
| ALT* | 46 (39) | 4 (3) | 22 (27) | 3 (5) |
| ALP | 53 (45) | 0 | 7 (12) | 0 |
| AST | 57 (48) | 1 (1) | 13 (22) | 1 (2) |
| Bilirubin | 46 (39) | 1 (1) | 23 (39) | 1 (2) |

ALP denotes alkaline phosphatase, ALT alanine aminotransferase, AST aspartate aminotransferase, and TEAE treatment-emergent adverse event.
*N = 117 for ALT for patients treated with imetelstat.

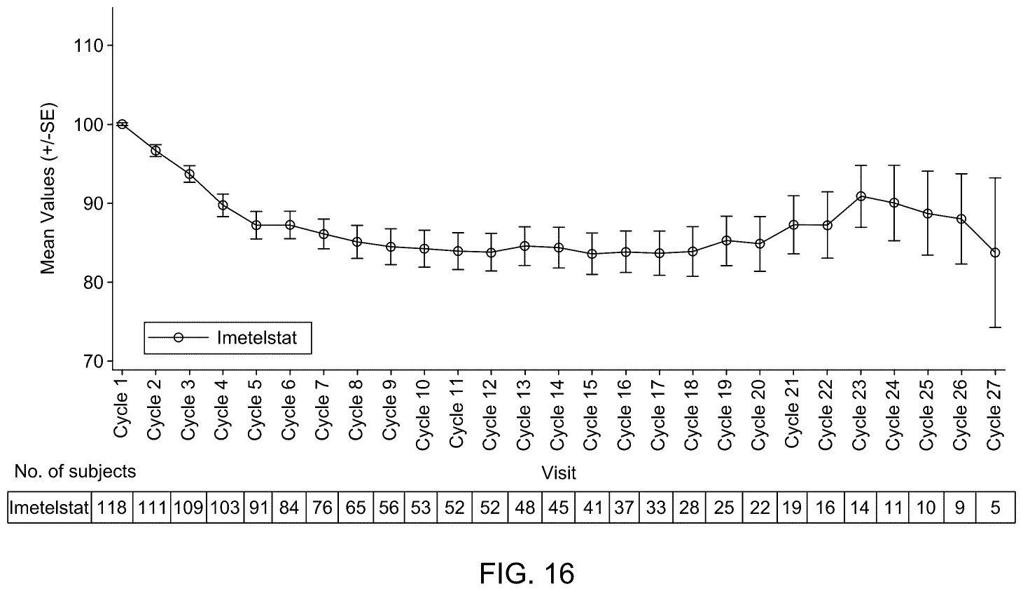

Fifty-eight patients (49%) receiving imetelstat and 4 patients (7%) receiving placebo had a dose reduction (Table 21; FIG. 16).

TABLE 21
Summary of infusion and dose modifications

| | Imetelstat | Placebo |
|---|---|---|
| n (%) | (N = 118) | (N = 59) |
| Patients with any infusion | 118 (100.0) | 59 (100.0) |
| Patients with any infusion or dose modification | 87 (73.7) | 18 (30.5) |
| Patients with any dose reduction | 58 (49.2) | 4 (6.8) |
| Patients with 2 dose reductions | 33 (28.0) | 1 (1.7) |
| Patients with any infusion interrupted/aborted/ | 10 (8.5) | 0 |
| rate change | | |
| Patients with any cycle delay | 86 (72.9) | 18 (30.5) |

Percentages calculated with the number of patients in each group as denominator. Cycle delay is defined as any interval >28 + 3 days between two cycles along with the investigator indicates cycle delay occurred.

Overall, 19 patients (16%) receiving imetelstat and none receiving placebo discontinued treatment because of AEs. AEs leading to treatment discontinuation included neutropenia (in 5% of patients) and thrombocytopenia (in 3% of patients; Table 22).

TABLE 22
TEAEs leading to withdrawal from treatment.

| System Organ Class | Imetelstat | Placebo |
|---|---|---|
| Preferred Term, n (%) | (N = 118) | (N = 59) |
| Patients experiencing TEAE leading to | 17 (14.4) | 0 |
| withdrawal from treatment | | |
| Blood and lymphatic system disorders | | |
| At least 1 TEAE | 10 (8.5) | 0 |
| Neutropenia | 6 (5.1) | 0 |
| Thrombocytopenia | 4 (3.4) | 0 |
| Anemia | 1 (0.8) | 0 |
| Cardiac disorders | | |
| At least 1 TEAE | 2 (1.7) | 0 |
| Atrial fibrillation | 1 (0.8) | 0 |
| Cardiac failure | 1 (0.8) | 0 |
| Neoplasms benign, malignant and unspecified | | |
| (including cysts and polyps) | | |
| At least 1 TEAE | 2 (1.7) | 0 |
| Lung neoplasm malignant | 1 (0.8) | 0 |
| Myelofibrosis | 1 (0.8) | 0 |
| Gastrointestinal disorders | | |
| At least 1 TEAE | 1 (0.8) | 0 |
| Gastrointestinal hemorrhage | 1 (0.8) | 0 |
| General disorders and administration | | |
| site conditions | | |
| At least 1 TEAE | 1 (0.8) | 0 |
| Asthenia | 1 (0.8) | 0 |
| Infections and infestations | | |
| At least 1 TEAE | 1 (0.8) | 0 |
| Renal abscess | 1 (0.8) | 0 |
| Sepsis | 1 (0.8) | 0 |
| Injury, poisoning and procedural complications | | |
| At least 1 TEAE | 1 (0.8) | 0 |
| Joint dislocation | 1 (0.8) | 0 |
| Psychiatric disorders | | |
| At least 1 TEAE | 1 (0.8) | 0 |
| Disorientation | 1 (0.8) | 0 |
| Respiratory, thoracic and mediastinal disorders | | |
| At least 1 TEAE | 1 (0.8) | 0 |
| Dyspnea | 1 (0.8) | 0 |
| Skin and subcutaneous tissue disorders | | |
| At least 1 TEAE | 1 (0.8) | 0 |
| Pruritus | 1 (0.8) | 0 |

Treatment-emergent adverse events (TEAE) include events that 1) occur after the first dose of study drug, through the treatment phase, and for 30 days following the last dose of study drug or until subsequent anticancer therapy if earlier; 2) any event that is considered study drug-related regardless of the start date of the event; or 3) any event that is present at baseline but worsens in severity or is subsequently considered drug-related by the investigator.

Seven patients (6%) in the imetelstat group and five patients (8%) in the placebo group discontinued treatment due to progressive disease: 2 patients (2%) in the imetelstat group and 1 patient (2%) in the placebo group due to acute myeloid leukemia progression, respectively. A total of 27 patients died during the trial: 19 patients (16%) in the imetelstat group and 8 patients (13%) in the placebo group, of which only 1 death in each group occurred within the treatment period (1 patient on imetelstat due to neutropenic sepsis study drug and 1 patient on placebo due to aortic stenosis, both not related to study drug).

### Example 2: Clinical Benefit of Imetelstat Treatment: Sustained, Meaningful Improvement in Fatigue

Patients with LR-MDS and anemia experience severe fatigue that negatively impacts overall functioning and daily life. Fatigue can also be a commonly reported adverse event with treatments for LR-MDS, the goals of which are to minimize transfusions and improve patient reported outcomes (PRO). In the Phase 3 study for imetelstat described in Example 1, patient reported fatigue (rate of deterioration/improvement) during treatment with imetelstat or placebo was evaluated.

To measure patient-reported fatigue, data were collected using the validated Functional Assessment of Chronic Illness Therapy (FACIT) Scale and analyzed in all randomized patients with available fatigue data at baseline (PRO population). The FACIT is a 13-item questionnaire measuring fatigue during daily activity. Proportion of sustained, meaningful deterioration/improvement was defined as percentage of pts with ≥3-point decrease/increase on the FACIT Fatigue Scale (0-52) for ≥2 consecutive treatment cycles. Additionally, time-to-deterioration/improvement was estimated by Kaplan-Meier analysis. Sensitivity analyses were performed in the intent-to-treat population and with alternate definitions of meaningful deterioration.

### Results:

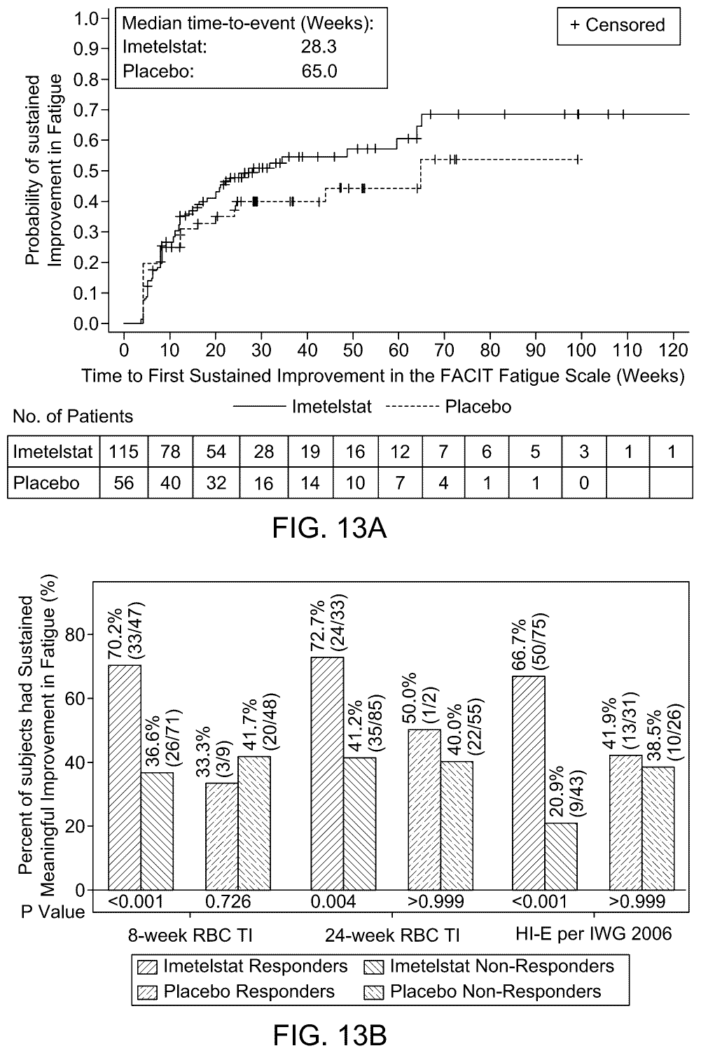

The PRO population included 118 patients on imetelstat and 57 patients on placebo. Completion rates for all PRO items were above 80% at most visits throughout the study in both groups. At baseline, mean age was 71 years (range, 39-87), Eastern Cooperative Oncology Group performance status was 0 (36.0%), 1 (60.6%), or 2 (3.4%), and 62.3% of patients were male. The proportion of patients who experienced any episode of sustained, meaningful deterioration in fatigue during this study was 43.2% in the imetelstat group and 45.6% in the placebo group. Results of sensitivity analyses were similar to the main analysis. Overall, 50.0% of patients in the imetelstat group reported sustained, meaningful improvement in fatigue vs 40.4% of pts in the placebo group. A shorter median time to reported first sustained, meaningful improvement in fatigue occurred with imetelstat vs placebo; 28.3 vs 65.0 weeks, HR=1.34 (95% CI, 0.82-2.20). After 12 weeks, more patients on imetelstat reported improvement in the FACIT Fatigue Scale than on placebo (FIG. 13A). Additionally, subsequent analysis demonstrated that ≥8 week and >24 week TI and HI-E per IWG 2006 criteria responses are highly significant and consistently correlated with sustained, meaningful improvement in fatigue scores in imetelstat treated subjects, but not those in the placebo group (FIG. 13B).

### Conclusion:

In this heavily transfusion-dependent (TD) population, both imetelstat- and placebo-treated patients reported similar rates of deterioration in fatigue, suggesting imetelstat did not worsen the rate of deterioration, which has been reported with currently available treatments. After 12 weeks, greater improvement of fatigue was reported with imetelstat compared to placebo; patients on imetelstat were more likely to have sustained, meaningful improvement in fatigue and quicker to experience it than those on placebo. Finally, the clinical benefit of imetelstat treatment is demonstrated by a robust correlation between TI and HI-E responses and sustained, meaningful improvement in fatigue.

### Example 3: Additional Information about the Phase 3 Trial

The following table (Table 23) provides a list of primary, secondary, and exploratory end points, assessed in the study described in the Examples described herein.

TABLE 23
List of Primary, Secondary, and Exploratory End Points Assessed in the Study.

| End Point | Method of Measurement | Study Day(s) Measured |
|---|---|---|
| Primary end point | | |
| Rate of RBC-TI for ≥8 | Transfusion history and status* | Up to 28 days before randomization; During |
| wks | | treatment phase: C1D1, C2D1, C3D1 and |
| | | each visit for study drug administration until |
| | | EOT; EOT visit; Posttreatment follow up.† |
| | | Each disease evaluation visit, all unscheduled |
| | | visits |
| Secondary end points | | |
| Safety of imetelstat | Incidence, intensity, and type of | Monitored continuously from screening to |
| | AEs, vital signs measurements, | EOT visit and Posttreatment follow up.† |
| | clinical laboratory values, ECG | |
| | changes, deaths | |
| 24-wk RBC-TI rate | Transfusion history and status* | Up to 28 days before randomization; During |
| | | treatment phase: C1D1, C2D1, C3D1, and |
| | | each visit for study drug administration until |
| | | EOT; EOT visit; Posttreatment follow up.† |
| | | Each disease evaluation visit, all unscheduled |
| | | visits |
| Time to 8-wk (24-wk) | Transfusion history and status* | Up to 28 days before randomization; During |
| RBC-TI | | treatment phase: C1D1, C2D1, C3D1 and |
| | | each visit for study drug administration until |
| | | EOT; EOT visit; Posttreatment follow up.† |
| | | Each disease evaluation visit, all unscheduled |
| | | visits |
| | | From Study Day 1 to the first day of the first |
| | | 8-wk (24-wk) RBC-TI period |
| Duration of RBC-TI | Transfusion history and status* | Up to 28 days before randomization; During |
| | | treatment phase: C1D1, C2D1, C3D1 and |
| | | each visit for study drug administration until |
| | | EOT; EOT visit; Posttreatment follow up.† |
| | | Each disease evaluation visit, all unscheduled |
| | | visits |
| Rate of hematologic | Hematology panel (local | Up to 28 days before randomization; During |
| improvement, | laboratory^{∥}); Hematology panel | treatment: C1D1; C1D8, C1D15, C1D22; |
| including HI-E, per | (central laboratory^{¶}); | C2D1, C2D8, C2D15, C2D22; C3D1 - until |
| modified IWG 2006 | Transfusion history and status* | EOT; EOT visit; Each disease evaluation visit, |
| | | all unscheduled visits; Posttreatment follow- |
| | | up (first visit, if feasible)+ |
| Rate of hematologic | Hematology panel (local | Up to 28 days before randomization; During |
| improvement, | laboratory^{∥}); Hematology panel | treatment: C1D1; C1D8, C1D15, C1D22; |
| including HI-E, per | (central laboratory^{¶}); | C2D1, C2D8, C2D15, C2D22; C3D1 - until |
| revised IWG 2018 | Transfusion history and status* | EOT; EOT visit; Each disease evaluation visit, |
| | | all unscheduled visits; Posttreatment follow- |
| | | up (first visit, if feasible)+ |
| Rates of CR, PR, or | Hematology panel (local | At screening; every 24 wks after C1D1, at |
| mCR per modified | laboratory^{∥}); Hematology panel | time of suspected CR, PR, or mCR and every |
| IWG 2006 | (central laboratory^{¶}) | 24 wks thereafter up to and including |
| | Bone marrow aspirate, response | suspected PD; manual absolute peripheral |
| | assessment †† | blast count for the central laboratory should be |
| | | done prior to randomization and on D1 of |
| | | each cycle only (aspirate); every 12 wks from |
| | | C1D1 (+1 wk) up to wk 72, then every 24 wks |
| | | until suspected PD and posttreatment follow- |
| | | up (response assessment) (first visit, if |
| | | feasible) |
| Overall survival | Follow-up | Monitored continuously from screening, |
| | | survival time of living patients will be |
| | | censored on the last day a patient is known to |
| | | be alive or lost to follow-up |
| Progression-free | Physician monitoring, follow up/ | At screening; every 24 wks after C1D1, at |
| survival | assessment of bone marrow | time of suspected CR, PR, or mCR and every |
| | aspirate, response assessment | 24 wks thereafter up to and including |
| | ††, hemoglobin, transfusion | suspected PD; manual absolute peripheral |
| | history* per IWG 2006 | blast count for the central laboratory should be |
| | definition of disease | done prior to randomization and on D1 of |
| | progression | each cycle only (aspirate); every 12 wks from |
| | | C1D1 (±1 wk) up to wk 72, then every 24 wks |
| | | until suspected PD and posttreatment follow- |
| | | up (response assessment). For patients who |
| | | do not have documented disease progression |
| | | and who are still alive at the end of the study |
| | | or clinical cutoff will be censored at the last |
| | | disease evaluation day |
| Time to progression to | Physician monitoring, follow up/ | At screening; every 24 wks after C1D1, at |
| AML | assessment of bone marrow | time of suspected CR, PR, or mCR and every |
| | aspirate, response assessment | 24 wks thereafter up to and including |
| | ††, hemoglobin, transfusion | suspected PD; manual absolute peripheral |
| | history* per IWG 2006 | blast count for the central laboratory should be |
| | definition of disease | done prior to randomization and on D1 of |
| | progression | each cycle only (aspirate); every 12 wks from |
| | | C1D1 (±1 wk) up to wk 72, then every 24 wks |
| | | until suspected PD and posttreatment follow- |
| | | up (response assessment). For patients who |
| | | have not progressed to AML and are still alive |
| | | at the cutoff date for the analysis or who |
| | | withdraw from the study (withdrawal of |
| | | consent or lost to follow-up), data will be |
| | | censored at the date of the last disease |
| | | evaluation |
| Amount and relative | Transfusion history and status* | Up to 28 days before randomization; During |
| change in RBC | | treatment phase: C1D1, C2D1, C3D1 and |
| transfusions | | each visit for study drug administration until |
| | | EOT; EOT visit; Posttreatment follow upf. |
| | | Each disease evaluation visit, all unscheduled |
| | | visits |
| Rate and duration of | Transfusion history and status*; | Up to 28 days before randomization; During |
| myeloid growth | myeloid growth factor | treatment phase: C1D1, C2D1, C3D1 and |
| factors usage | treatment | each visit for study drug administration until |
| | | EOT; EOT visit; Posttreatment follow upf. |
| | | Each disease evaluation visit, all unscheduled |
| | | visits |
| PK parameters and | Plasma sample assessment | C1D1; D1 of all subsequent cycles (PK); |
| immunogenicity of | | C4D1, C7D1, C10D1 (immunogenicity); if |
| imetelstat | | IRR is observed (PK); EOT visit |
| | | (immunogenicity); first posttreatment follow- |
| | | up, if feasible (immunogenicity) |
| Medical resource | Data collection | Continuous during treatment phase, EOT visit |
| utilization data, | | |
| including | | |
| hospitalization, | | |
| emergency room visits, | | |
| and hematology | | |
| specialist visits | | |
| Assessment of | Patient reporting before any | C1D1; C2D1; C3 until EOT D1; EOT visit; |
| QUALMS, FACT-An, | tests, procedures, dosing or | posttreatment follow-up‡§ |
| and EQ-5D-5L | other consultations are | |
| | performed and done before the | |
| | patient is informed of any | |
| End Point | Method of Measurement | Study Day(s) Measured |
| | assessment results, when | |
| | possible | |
| Exploratory end | | |
| points | | |
| TA and hTERT at | Peripheral blood sample | TL: C1D1 (predose); TA and hTERT: C1D1 |
| baseline and the | analysis | (predose and 24 h postdose), C1D8, C2D1 |
| change in baseline, TL | | (predose), C2D8 |
| at baseline only | | |
| Cytogenetic status at | Bone marrow aspirate, response | At screening; aspirate is required for all |
| baseline and change | assessment ††, | |
| over time for | | patients with baseline abnormalities every 24 |
| cytogenetic response | | wks after C1D1, at the time of suspected PR, |
| | | or CR and every 24 wks thereafter up to and |
| | | including suspected PD |
| Mutation status at | Mutation analysis | C1D1; every 12 wks after C1D1, at time of |
| baseline and change | | suspected response HI-E, PR, mCR or CR and |
| over time, including at | | at time of PD |
| the time of suspected | | |
| response (CR, PR, | | |
| mCR, HI-E [Hb]) or | | |
| PD | | |

*Includes date of transfusion, no. of units and type of blood products, pre-transfusion Hb and platelet count; Enhanced and continuous medical review implemented to identify any sudden but unsustained increase of Hb values and to query whether transfusion had occurred.
†Every 4 to 6 wks until first transfusion in posttreatment follow-up.
‡Posttreatment follow-up until start of subsequent therapy.
§At all visits, complete before any tests, procedures, dosing or other consultations are performedand before the patient is informed of any assessment results, when possible.
^{∥}Screening tests should be within 14 days prior to Randomization. For C1D1, no need to repeat if Screening tests performed within 5 days. Refer to Inclusion Criteria for further details. After C1D1, tests must be performed within 48h before the scheduled visit. For all cycles, if tests are repeated on the day of the visit, the most recent results should be reviewed before dosing.
Perform unscheduled weekly follow-up after any Grade >3 thrombocytopenia or neutropenia until values return to baseline. Testing should be conducted pre-transfusion, if feasible.
Hematology panel includes hemoglobin, platelet count, white blood cell count, ANC. Absolute peripheral blast count should be done prior to randomization and on D1 of each cycle only.
Note:
if DE Visit does not align with a Cycle Day 1 visit, then hematology (local) is required at the DE Visit.
^{¶}Hematology panel assessed in central laboratory will be hemoglobin, platelet count, white blood cell count and ANC. Central hematology sample should also be collected pre-transfusion, if feasible. Manual absolute peripheral blast count for the central lab should be done prior to randomization and on D1 of each cycle only. Note: if DE Visit does not align with a Cycle Day 1.
††A BM aspirate and biopsy with iron stain completed up to 12 weeks prior to Randomization may be used for confirmation of eligibility (assessed both locally and centrally); otherwise, BM biopsy and aspirate assessment must be done during screening. After baseline, BM assessments may be performed with a window of ±7 days. Following investigator-assessed CR, PR, or mCR, in patients with >5% baseline bone marrow aspirate blasts, repeat aspirate 4 to 8 weeks later for confirmatory purposes, if clinically feasible. Central Pathology Review: Timely submission of all bone marrow samples (aspirate smear and bone marrow biopsy) to the Independent Central Pathology Reviewer is required for diagnostic confirmation. Local pathology assessments are used for treatment decisions. Each response assessment every 12 weeks requires review of peripheral blood and RBC transfusion requirements. In addition, bone marrow analysis (including central review) is required every 24 weeks with additional assessments at the time of suspected response or progression. Investigators will use local laboratory and bone marrow results for response evaluations. If study drug is discontinued for any reason other than disease progression.
Note:
CR, PR or mCR only assessed in patients with >5% baseline bone marrow aspirate blasts. AE denotes adverse event, AML acute myeloid leukemia, C Cycle, CR complete remission, D Day, ECG electrocardiogram, EOT end of treatment, EQ-5D-5L EuroQoL-5D-5L, FACT-An Functional Assessment of Cancer Therapy - Anemia-Related Effects, h hour, Hb hemoglobin, HI-E hematologic improvement-erythroid, hTERT human telomerase reverse transcriptase, IWG International Working Group, mCR marrow complete remission, MDS myelodysplastic syndrome(s), PD progressive disease/disease progression, PK pharmacokinetics, PR partial remission, QTcF QT interval by Fridericia's correction method, QUALMS Quality of Life in Myelodysplasia Scale, RBC red blood cell, RBC-TI RBC transfusion independence, TA telomerase activity, TL telomerase length, and wk, week.

Additional Supplementary Information about the Methods

### Study Inclusion Criteria

Man or woman age ≥18 years (or the legal age of consent in the jurisdiction in which the study took place):

In this phase 3 study, diagnosis of MDS according to WHO criteria was confirmed by bone marrow aspirate and biopsy within 12 weeks prior to Randomization. A sample of the baseline bone marrow aspirate and biopsy was confirmed to be submitted to the Independent Central Pathology Reviewer for diagnostic confirmation. Central laboratory was required to review to confirm diagnosis prior to Randomization.

IPSS low or intermediate-1 risk MDS:

RBC transfusion dependent, defined as requiring at least 4 RBC units transfused over an 8-week period during the 16 weeks prior to Randomization; pretransfusion Hb must have been ≤9.0 g/dL to count towards the 4 units total;

Had MDS that was relapsed/refractory to ESA treatment; as defined by meeting any one of the criteria below:

Received at least 8 weeks of treatment with a minimum weekly dose of epoetin alfa 40,000U, epoetin beta 30,000U or darbepoetin alfa 150 mcg (or equivalent agent/dose), without having achieved a Hb rise≥1.5 g/dL or decreased RBC transfusion requirement by at least 4 units over 8 weeks

Transfusion dependence or reduction in Hb by ≥1.5 g/dL after hematologic improvement from at least 8 weeks of treatment with therapies outlined in inclusion criteria as discussed in the preceding paragraph, in the absence of another explanation.

Endogenous serum EPO level>500 mU/mL:

Adequate iron stores, defined as transferrin saturation greater than 20% and serum ferritin greater than 400 ng/mL, measured within the screening period, or adequate iron stores as demonstrated by recent (within 12 weeks prior to Randomization) bone marrow examination with iron stain.

ECOG performance status 0, 1 or 2:

Hematology lab test values within the following limits:

ANC≥1.5×10⁹/L independent of growth factor support,

Platelets≥75×10⁹/L independent of platelet transfusion support.

Biochemical laboratory test values must be within the following limits:

AST, ALT and ALP≤2.5 times the upper limit of normal (×ULN).

Serum creatinine≤2.0×ULN.

Total bilirubin≤3×ULN and direct bilirubin≤2×ULN (unless due to Gilbert's syndrome, ineffective erythropoiesis due to MDS, or hemolysis due to RBC transfusion):

Women of childbearing potential and practicing a highly effective method of birth control consistent with local regulations regarding the use of birth control methods for patients participating in clinical studies: e.g., established use of oral, injected or implanted hormonal methods of contraception; placement of an intrauterine device or intrauterine system; barrier methods: condom with spermicidal foam/gel/film/cream/suppository or occlusive cap (diaphragm or cervical/vault caps) with spermicidal foam/gel/film/cream/suppository; male partner sterilization (the vasectomized partner should be the sole partner for that patient); true abstinence (when this is in line with the preferred and usual lifestyle of the patient). For females, these restrictions applied for 1 month after the end of dosing. If the childbearing potential changed after start of the study (e.g., woman who is not heterosexually active becomes active, premenarchal woman experiences menarche) a woman must have begun a highly effective method of birth control, as described above.

A woman of childbearing potential must have had a negative serum (β-human chorionic gonadotropin [β-hCG]) or urine pregnancy test at screening and agree to be tested on Day 1 of every cycle and at end of study (30 days post last dose).

A man who is sexually active with a woman of childbearing potential and has not had a vasectomy must have agreed to use a barrier method of birth control, e.g., either condom with spermicidal foam/gel/film/cream/suppository or partner with occlusive cap (diaphragm or cervical/vault caps) with spermicidal foam/gel/film/cream/suppository, and all men must have refrained from donating sperm during the study. For males, these restrictions applied for 3 months after the end of dosing.

Each patient (or their legally acceptable representative) signed an informed consent form (ICF) indicating that he or she understood the purpose of, and procedures required for, the study and were willing to participate in the study.

### Study Exclusion Criteria

Patients with known allergies, hypersensitivity, or intolerance to imetelstat or excipients of imetelstat drug formulations.

Patient who received an experimental or investigational drug or used an invasive investigational medical device within 30 days prior to C1D1 (Part 1) or Randomization (Part 2) (defined in Section 3.1) or is currently enrolled in an investigational study.

Prior treatment with imetelstat:
Received corticosteroids>30 mg/day prednisone or equivalent, or growth factor treatment within 4 weeks prior to C1D1 (Part 1) or Randomization (Part 2);
Prior treatment with a hypomethylating agent (e.g., azacitidine, decitabine);
Prior treatment with lenalidomide, thalidomide, or other thalidomide analogues;
Received an ESA or any anti-MDS therapy, chemotherapy, immunomodulatory, or immunosuppressive therapy within 4 weeks prior to C1D1 (Part 1) or Randomization (Part 2) (8 weeks for long-acting ESAs);
Prior history of hematopoietic stem cell transplant;
Anemia attributed to factors other than MDS (including hemolysis, chronic renal failure, hepatitis, gastrointestinal bleeding);
Major surgery within 4 weeks prior to C1D1 (Part 1) or Randomization (Part 2) (excluding the placement of vascular access and other minor surgical procedures);
Diagnosed or treated for malignancy other than MDS, except:

Malignancy treated with curative intent and with no known active disease present for ≥3 years before C1D1 (Part 1) or Randomization (Part 2).

Adequately treated nonmelanoma skin cancer or lentigo maligna without evidence of disease.

Adequately treated cervical carcinoma in situ without evidence of disease.

Clinically significant cardiovascular disease such as uncontrolled or symptomatic arrhythmias, congestive heart failure, or myocardial infarction within 6 months of C1D1 (Part 1) or Randomization (Part 2), or any Class 3 (moderate) or Class 4 (severe) cardiac disease as defined by the New York Heart Association Functional Classification.

Known history of human immunodeficiency virus (HIV) or any uncontrolled active systemic infection requiring IV antibiotics.

Active systemic hepatitis infection requiring treatment (carriers of hepatitis virus were permitted to enter the study), or known acute or chronic liver disease including cirrhosis.

Females who were pregnant or were breastfeeding or planning to become pregnant while enrolled in this study or within 1 month after the end of dosing.

Patient was a man who plans to father a child while enrolled in this study or within 3 months after the end of dosing.

Any life-threatening illness, medical condition, or organ system dysfunction which, in the investigator's opinion, could compromise the patient's safety, interfere with the imetelstat metabolism, or put the study outcomes at undue risk; Patient had any condition for which, in the opinion of the investigator, participation would not be in the best interest of the patient (e.g., compromise the well-being) or that could prevent, limit, or confound the protocol-specified assessments.

Patient was previously assessed as having IPSS intermediate-2 or high risk MDS.

Patient with del(5q) karyotype.

Patient with MDS/myeloproliferative neoplasm Overlap Syndrome.

### HI-E Per IWG 2006 Criteria

Proposed modified International Working Group response criteria for hematologic improvement is shown in Table 24.

TABLE 24
Proposed modified International Working Group
response criteria for hematologic improvement.

| Hematologic | |
|---|---|
| Improvement* | Response criteria (responses must last >8 weeks)+ |
| Erythroid response | Hb increase by ≥1.5 g/dL |
| (pretreatment, <11 g/dL) | Relevant reduction of units of RBC transfusions by an absolute |
| | number of at least 4 RBC transfusion units/8 weeks compared |
| | with the pretreatment transfusion number in the previous 8 weeks. |
| | Only RBC transfusions given for a Hb of ≤9 g/dL pretreatment |
| | will count in the RBC transfusion response evaluation+ |
| Platelet response | Absolute increase of ≥30 × 10⁹/L for patients starting with >20 × |
| (pretreatment, <100 × | 10⁹/L platelets |
| 10⁹/L) | Increase from <20 × 10⁹/L to >20 × 10⁹/L and by at least 100%† |
| Neutrophil response | At least 100% increase and an absolute increase >0.5 x 109/L† |
| (pretreatment, <1 × | |
| 10⁹/L) | |
| Progression or relapse | At least 1 of the following: |
| after HI‡ | At least 50% decrement from maximum response levels in |
| | granulocytes or platelets |
| | Reduction in Hb by ≥1.5 g/dL |
| | Transfusion dependence |

*Pretreatment counts averages of at least 2 measurements (not influenced by transfusions) ≥1 week apart (modification).
†Modification to IWG response criteria.
‡In the absence of another explanation such as acute infection, repeated courses of chemotherapy (modification), gastrointestinal bleeding, hemolysis, and so forth. It is recommended that 2 kinds of erythroid and platelet responses be reported overall as well as by the individual response pattern.
Deletions to the IWG response criteria are not shown. To convert hemoglobin levels (concentrations) from grams per deciliter to grams per liter, multiply grams per deciliter by 10.
CR denotes complete remission,; DFS disease-free survival, FAB French-American-British, Hb hemoglobin, HI hematologic improvement, IWG International Working Group, MDS myelodysplastic syndromes, PFS progression-free survival, PR partial remission, RBC red blood cell.
Source of HI-E per IWG 2006 Criteria: Cheson BD, Greenberg PL, Bennett JM, et al. Clinical application and proposal for modification of the International Working Group (IWG) response criteria in myelodysplasia. Blood 2006; 108: 419-25.

### HI-E Per IWG 2018 Criteria2

Suggested modified International Working Group response criteria for response evaluation is provided in Table 25 below.

TABLE 25
Suggested modified International Working Group
response criteria for response evaluation

| Response evaluation | Response criteria |
|---|---|
| NTD (RBCs in 16 | At least 2 consecutive Hb measurements ≥1.5 g/dL for a period of |
| weeks)* | minimum 8 weeks in an observation period of 16 to 24 weeks |
| | compared with the lowest mean of 2 Hb measurements (apart from |
| | any transfusion) within 16 weeks before treatment onset^{†} |
| | Response duration of at least 16 wk is considered clinically |
| | meaningful |
| LTB (3-7 RBCs in 16 | HI-E in patients with LTB corresponds to transfusion |
| weeks in at least 2 | independence, defined by the absence of any transfusions for at |
| transfusion episodes, | least 8 weeks in an observation period of 16-24 weeks with the |
| maximum 3 in 8 weeks) | same transfusion policy compared with 16 weeks prior to treatment |
| | Response duration of at least 16 wk, however, is considered |
| | clinically meaningful |
| HTB (≥8 RBCs in 16 | Major response: Major HI-E response in patients with HTB |
| weeks, ≥4 in 8 weeks) | corresponds to transfusion independence, defined by the absence |
| | of any transfusions over a period of minimum 8 wk in an |
| | observation period of 16-24 weeks with the same transfusion |
| | policy (defined below) compared with 16 weeks prior to treatment; |
| | only a response duration of at least 16 weeks, however, is |
| | considered clinically meaningful |
| | Minor response: Minor HI-E response in patients with HTB is |
| | defined as a reduction by at least 50% of RBCs over a minimum of |
| | 16 weeks with the same transfusion policy (defined below) |
| | compared with 16 weeks prior to treatment |
| On-treatment RBC | Transfusion policy for the individual patient prior to therapy |
| transfusion policy^{‡} | should be maintained on treatment if not otherwise clinically |
| | indicated |
| | Maximum variation between pre- and on-study practice of 1 g/dL |
| | (or 0.6 mmol/L) in terms of transfusion threshold |
| Dose adjustment | If the drug under investigation is stopped or its dose reduced in a |
| thresholds for high Hb | responding patient for protocol-defined reasons leading to a loss of |
| levels | response, this should not be counted as such if reintroduction at the |
| | same or lower dose of the drug induces a new response |
| | Reintroduction of the drug at a lower dose does not reinduce a |
| | response, this should be documented as such |

†Oscillations (e.g., natural or due to drug intervals) within this period are accepted as long as the patient remains off any transfusions and the same transfusion policy has been maintained. We suggest accepting 1 drop to an increase of between 1.0 and 1.5 g/dL over a period of 8 wk. We recommend that intervals between blood counts do not exceed 2 wk.
^{‡}Exceptions to this rule may be accepted in cases of well-documented moderate or severe angina pectoris, cardiac or pulmonary insufficiency, or ischemic neurologic diseases. In these cases, a higher transfusion trigger level may be established for an individual patient. These patients may require special attention when analyzing responses within clinical trials. Transfusions for intercurrent diseases (bleeding, surgical procedure, etc.) should not be considered.
HI-E denotes hematologic improvement-erythroid,; hemoglobin HI hematologic improvement, HTB high red blood cell transfusion burden, IWG International Working Group, LTB low red blood cell transfusion burden, RBC red blood cell.

### End Points Definitions

The 8-week RBC-TI rate was the proportion of patients without any RBC transfusion during any consecutive 8 weeks (56 days) starting from Study Day 1 until subsequent anti-cancer therapy, if any. The starting date of the 8-week period of transfusion independence was between randomization and 30 days after the last dose, the end-of-treatment-visit, or 31 days after randomization if randomized but not treated. Study Day 1 is defined as the day of randomization for patients enrolled in the Phase 3 study.

Key Secondary End Point TI Rates was the proportion of patients without any RBC transfusion during any consecutive 16 weeks, 24 weeks, or 1 year, starting from Study Day 1.

Key Secondary End Point Duration of RBC-TI was the first day of the longest RBC-TI period to the date of the first RBC transfusion after the TI period starts.

Key Secondary End Point Rate of hematologic improvement, including HI-E: per modified IWG 2006.

Key Secondary End Point Complete remission (CR), Partial remission (PR), or Marrow complete remission (mCR): Proportion of patients who achieve CR, PR, or mCR per modified IWG 2006.

Exploratory End Point: Mutation status at baseline and change over time, assessed including at the time of suspected response (CR, PR, mCR, HI-E [Hb]) or progressive disease.

Exploratory End Point Patient-Reported Fatigue: Proportion of sustained meaningful deterioration/improvement was defined as percentage of patients with ≥3-point decrease/increase on the Functional Assessment of Chronic Illness Therapy (FACIT) Fatigue Scale (0-52) for ≥2 consecutive treatment cycles.

CR denotes complete remission; Hb hemoglobin, HI hematologic improvement, HI-E hematologic improvement-erythroid, IWG International Working Group, PR partial remission, RBC red blood cell.

### Additional Statistical Methods

Changes over time in transfusion units and hemoglobin levels were compared by a mixed model for repeated measures with weeks since randomization, prior transfusion burden, minimum hemoglobin in the 8 weeks prior to first dose (for hemoglobin), and treatment arm as the independent variables with autoregressive moving average covariance structure.

Meaningful improvement in FACIT Fatigue: Psychometric supportive evidence for the use of the FACIT Fatigue in the context of LR-MDS confirms the previously published threshold of a 3-point change for meaningful within-patient change for the FACIT Fatigue.

FACIT denotes Functional Assessment of Chronic Illness Therapy; LR-MDS lower risk myelodysplastic syndromes.

### Gene Mutation Analysis

Blood samples were collected at baseline and during study visits approximately every 12 weeks. Following DNA extraction from leukocytes, a targeted, amplicon-based next generation sequencing was performed at Quest Diagnostics using DNA bait capture methodology on the NextSeq® (Illumina®) platform and a LeukoVantage® MDS Gene Panel covering 36 MDS-relevant genes. The lower limit of sensitivity is 5% mutated alleles in a mixed population; the percentage of mutation reads is reported and used to assess the size of the clonal population.

Baseline mutation data was available from 165 of 178 patients enrolled in the study (n=110 in the imetelstat group; n=55 in the placebo group). Mutated genes with frequency >10% in this study are listed in Table 26.

TABLE 26
Baseline mutational profile.

| Patients, n (%) | Imetelstat (n = 110) | | Placebo (n = 55) | |
|---|---|---|---|---|
| ≥1 mutation | 107 | (97.3) | 54 | (98.2) |
| >1 mutation | 75 | (68.2) | 38 | (69.1) |
| >2 mutations | 33 | (30.0) | 15 | (27.3) |
| >3 mutations | 9 | (8.2) | 7 | (12.7) |
| SF3B1 mutations | 82 | (74.5) | 43 | (78.2) |
| TET2 mutations | 40 | (36.4) | 14 | (25.5) |
| DNMT3A mutations | 19 | (17.3) | 9 | (16.4) |
| ASXLI mutations | 18 | (16.4) | 6 | (10.9) |
| CUXI mutations | 14 | (12.7) | 7 | (12.7) |
| RNA spliceosome | 96 | (87.3) | 47 | (85.5) |
| Epigenetic modifiers | 69 | (62.7) | 29 | (52.7) |
| Transcription regulation | 10 | (9.1) | 9 | (16.4) |
| Receptors/kinases | 5 | (4.5) | 2 | (3.6) |
| Poor-prognosis genes | 22 | (20.0) | 11 | (20.0) |

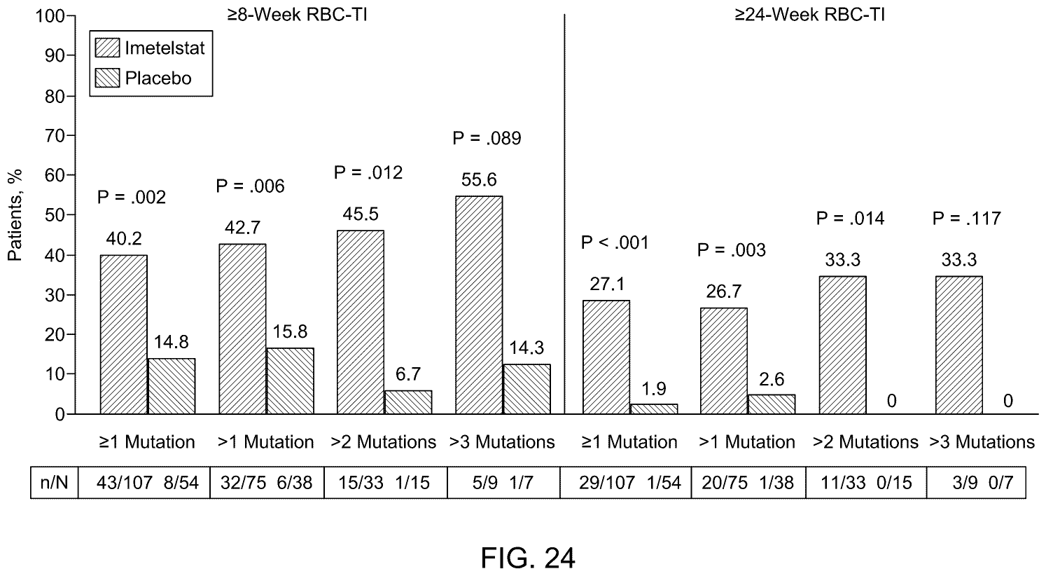

RBC-TI by number of baseline mutations: In patients who had ≥1 mutation detected, imetelstat significantly improved the 8-week (P=0.002) and 24-week (P<0.001) RBC-TI response rates compared with placebo (FIG. 24).

Significant rate differences were also noted in patients who had >2 mutations at baseline: 45.5% vs 6.7% for ≥8-week RBC-TI (P=0.012) and 33.3% vs 0% for ≥24-week RBC-TI (P=0.014) with imetelstat vs placebo, respectively.

Of patients with mutation data, 161 (97.6%) had ≥1 mutation detected: 107 patients (97.3%) in the imetelstat-treated group and 54 patients (98.2%) in the placebo-treated group. Proportions of frequently occurring mutations were well balanced between the treatment groups (Table 26).

SF3B1 mutations were detected at baseline in nearly 75% of the patients in the imetelstat-treated group and 78% of those in the placebo-treated group. Poor-prognosis mutations were identified in 20% of samples in either treatment group: each group had 2 patients with TP53 and 2 patients with RUNX1 mutations, and there were 18 vs 6 patients with ASXL1 mutations, 2 vs 1 patient with ETV6 mutations, and 0 vs 2 patients with EZH2 in the imetelstat-vs placebo-treated groups, respectively.

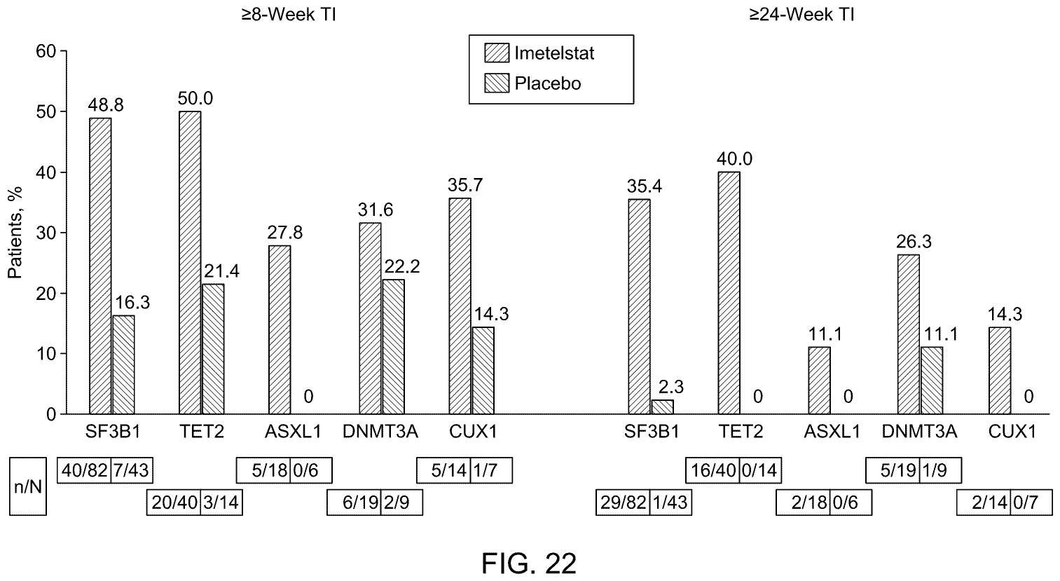

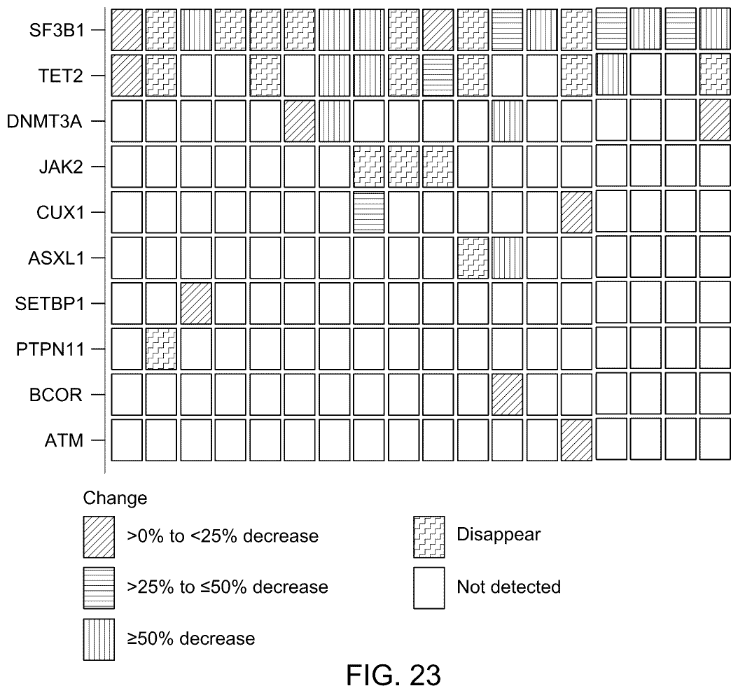

Among patients assessed for genes commonly mutated in MDS, those harboring SF3B1 mutations at baseline had significantly higher rates of RBC-TI responses with imetelstat vs placebo at both 8 weeks (48.8% vs 16.3%; P=0.001) and 24 weeks (35.4% vs 2.3%; P<0.001) as shown in FIG. 22. Similar trends were seen with the other commonly mutated genes (FIG. 22) and in patients with SF3B1 hot-spot mutations (≥2 patients in either group), albeit the sample size was small (Table 27).

TABLE 27
Summary of TI response rates by baseline mutation
status of SF3B1 hot-spot mutations.

| | ≥8-Week RBC-TI | | | | ≥24-Week RBC-TI | | | |
|---|---|---|---|---|---|---|---|---|
| Patients, | Imetelstat | | Placebo | | Imetelstat | | Placebo | |
| n/N (%) | (n = 82) | | (n = 43) | | (n = 82) | | (n = 43) | |
| E622D | 2/8 | (25.0) | 1/2 | (50.0) | 2/8 | (25.0) | 1/2 | (50.0) |
| R625C/L/G | 4/7 | (57.1) | 0/5 | (0) | 2/7 | (28.6) | 0/5 | (0) |
| H662Q/N/D/Y | 7/12 | (58.3) | 0/5 | (0) | 6/12 | (50.0) | 0/5 | (0) |
| T663P | 2/2 | (100) | 0 | | 2/2 | (100) | 0 | |
| K666R/T/Q/N | 2/6 | (33.3) | 0/7 | (0) | 0/6 | (0) | 0/7 | (0) |
| K700E | 18/41 | (43.9) | 5/22 | (22.7) | 12/41 | (29.3) | 0/22 | (0) |

| | | | | | | |
|---|---|---|---|---|---|---|
| A744P | 2/2 | (100) | 0 | 2/2 | (100) | 0 |
| E783K | 1/2 | (50.0) | 0 | 1/2 | (50.0) | 0 |

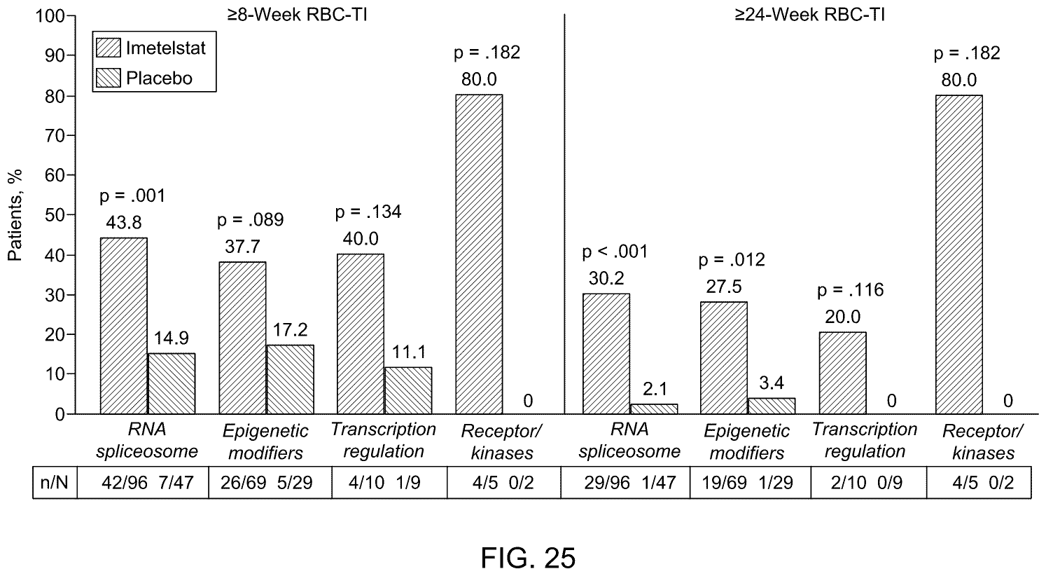

RBC-T rates by baseline mutation status of 4 sets of genes involved in different biological functions show that consistent with the presence of baseline SF3B1 mutations, patients with mutations in genes regulating RNA spliceosome had significantly higher rates of 8-week and >24-week TI responses with imetelstat than with placebo: 43.8% vs 14.9% (P=0.001) and 30.2% vs 2.1% (P<0.001), respectively (FIG. 25). Similar trends were noted with the other gene sets, albeit there was no significant difference between groups.

RBC-TI rates by baseline mutation status of poor-prognosis genes show that imetelstat treatment indicated higher ≥8-week (Table 28) and >24-week RBC-TI response rates versus placebo in patients with poor-prognosis genes mutated at baseline: 31.8% vs 0% and 9.1% vs 0%, respectively.

TABLE 28
Summary of 8-week TI response rates by baseline
mutation status of poor-prognosis genes.

| | | |
|---|---|---|
| Patients, n/N (%) | Imetelstat (n = 22) | Placebo (n = 11) |

| | | | |
|---|---|---|---|
| Poor prognosis | 7/22 | (31.8) | 0/11 (0) |
| ASXLI mutations | 5/18 | (27.8) | 0/6 (0) |
| TP53 mutations | 2/2 | (100) | 0/2 (0) |
| ETV6 mutations | 1/2 | (50.0) | 0/1 (0) |
| RUNXI mutations | 0/2 | (0) | 0/2 (0) |

| | | |
|---|---|---|
| EZH2 mutations | 0 | 0/2 (0) |

These data show that, overall, in patients with various baseline mutational profiles, imetelstat treatment led to higher TI rates than placebo. A significantly higher percentage of imetelstat-treated than placebo-treated patients with baseline mutations in SF3B1, a gene commonly mutated in MDS and involved in regulation of RNA spliceosome, achieved 8- and 24-week RBC-TI. RBC-TI responses in patients receiving imetelstat occurred regardless of the presence of mutations associated with poor prognosis or the number of mutations. RBC-TI responses with imetelstat were observed across different molecularly defined subgroups, suggesting that clinical benefit of imetelstat in patients with LR-MDS is independent of the underlying molecular pattern.

Heatmap of the changes in the mutational burden in 18 patients with ≥1 year TI is described in FIG. 23.

### Example 4: Imetelstat Provides Clinically Meaningful and Statistically Significant Improvement in RBC-TI Rates

This is the first trial showing clinically meaningful and statistically significant improvement in RBC-TI rates in patients with ESA relapsed, refractory, or ineligible non-del(5q) LR-MDS treated with imetelstat, a telomerase inhibitor, compared with patients treated with placebo. Among 8-week RBC-TI responders in imetelstat group, RBC-TI was durable (median duration 52 weeks) and consistent over time, with 83% of patients experiencing a single continuous RBC-TI period. Twenty-eight and eighteen percent of patients in imetelstat group achieved RBC-TI for at least 24 weeks and 1 year, respectively. Significant improvement in RBC-TI was observed with imetelstat versus placebo across different LR-MDS subgroups, including patients with or without RS and in patients with high transfusion burdens.

For the key secondary end point of HI-E based on the modified IWG 2006 criteria, the difference in the improvement rate for imetelstat versus placebo was not statistically significant. However, when HI-E was assessed using the more recent IWG 2018 criteria, which prioritizes durability by measuring response for ≥16 weeks rather than ≥8 weeks, the effect of imetelstat was significant and more profound. Patients treated with imetelstat also demonstrated significantly higher increase in hemoglobin levels over time, a biological measure that would be expected to lead to the clinical end point of RBC-TI.

These results also correspond to improved patient-reported fatigue in patients receiving imetelstat; imetelstat-treated patients were more likely to experience sustained, meaningful improvement in fatigue with shorter median time to improvement than those on placebo. This improvement in fatigue was not observed in other pivotal studies of treatment for LR-MDS, including luspatercept. A significant association between RBC-TI and sustained meaningful improvement in fatigue further supports the clinical benefit of imetelstat treatment.

The mutant allele burden of SF3B1, TET2, ASXL1, and DNMT3A reflects the size and disease burden of the malignant clonal population in LR-MDS. The maximum percent reduction in mutation VAF was higher in patients treated with imetelstat compared with those treated with placebo, and a reduction in VAF correlated with RBC-TI rates, RBC-TI duration and hemoglobin increase. Both clinical and molecular evidence indicates that imetelstat may restore functional erythropoiesis by reducing or eliminating malignant clones, supporting a potential for MDS disease modification.

Safety results were consistent with previous clinical experience with imetelstat and no new safety signals were reported. With dose delays and dose reduction to manage the common adverse events of neutropenia and thrombocytopenia, efficacy was maintained, suggesting that these events can be adequately managed by treating physicians.

These cytopenias were the most common treatment-emergent AEs and the majority occurred early in treatment with imetelstat. Transient thrombocytopenia can represent an on-target effect of imetelstat treatment, resulting from the differential impact on malignant stem and progenitor cells, and the subsequent recovery of blood cell production. Importantly, due to reversibility and short onset, the clinical consequences of grade 3-4 bleeding events, infections, and febrile neutropenia were similar in both treatment groups, indicating no increased risk of these severe events.

One potential trial limitation was that investigators were instructed to follow local standards regarding clinical criteria for when patients should receive transfusions. Differing local standards may have resulted in variability in the sequelae that prompted transfusions for patients across the trial; however, this variability was consistent across both arms and reflects treatment of LR-MDS in real-world settings. In conclusion, imetelstat demonstrated durable RBC-TI, significant reduction in transfusion burden, and increased hemoglobin levels in patients with LR MDS who were relapsed, refractory or ineligible for ESA treatment. Importantly, robust activity irrespective of RS and mutational status and in patients with high transfusion burdens distinguish imetelstat from currently available therapies. Furthermore, durable RBC-TI for 24 weeks and even longer than 1 year have not been previously demonstrated. Improvement in fatigue, cytogenetic responses, and the reduction of VAF and their correlation to clinical end point of transfusion independence support the disease-modifying potential of imetelstat. Taken together, IMerge phase 3 results validate the observations from the phase 2 part and demonstrate that imetelstat provides significant clinical benefit to a heavily TD LR-MDS patient population.

### Example 5: Characteristics and Clinical Benefit for Imetelstat Receiving Patients with Sustained TI For ≥1 Year

In the IMerge phase 3 trial, imetelstat produced higher rates of TI for ≥8 weeks, ≥24 weeks, and >1 year (39.8%, 28.0%, and 17.8%) than placebo (15.0%, 3.3%, and 1.7%) in patients with non-del(5q) LR-MDS that was RBC-TD, R/R to/ineligible for ESAs, and naïve to lenalidomide or hypomethylating agents (HMAs). Characteristics and clinical benefits for patients with sustained TI for ≥1 year from this trial are reported.

Results: Of 118 pts receiving imetelstat, 21 (17.8%, 95% CI [11.4%-25.9%]) achieved ≥1-year sustained TI, representing 45% of ≥8-week TI (21 of 47 pts) and 64% of ≥24-week TI (21 of 33 pts). Of 60 patients receiving placebo plus supportive care, 1 (1.7%) achieved ≥1-year TI. Of the ≥1-year TI imetelstat responders, 15/21 (71.4%) had ring sideroblasts, as did the one placebo patient. The median prior RBC transfusion burden was 6U over 8 weeks (range, 4-9U) for the imetelstat group and 5U for the placebo patient. Central hemoglobin improved to a median of 5.2 g/dL in ≥1-year imetelstat-treated responders. Additional baseline characteristics are provided in the Table 29.

TABLE 29
Baseline characteristics of IMerge phase
3 LR-MDS patients achieving TI ≥ 1 year.

| | | |
|---|---|---|
| | Imetelstat | Placebo |
| Baseline Characteristic | (N = 21) | (N = 1) |

| IPSS category | | | | |
|---|---|---|---|---|
| Low | 14 | (67) | 1 | (100) |
| Intermediate-1 risk | 7 | (33) | 0 | |
| MDS-RS+ | 15 | (71) | 1 | (100) |

IPSS-R category

| Very Low, low | 15 | (71) | 1 | (100) |
|---|---|---|---|---|
| Intermediate | 4 | (19) | 0 | |
| Missing | 2 | (10) | 0 | |
| Prestudy RBC transfusion | 6.0 | (4-9) | 5.0 | |
| burden, median (range), ^{a}U | | | | |
| Prior ESAs | 19 | (90) | 1 | (100) |

≥2 years since initial diagnosis

| Pretreatment hemoglobin level, | 7.8 | (6.5-8.8) | 6.2 | |
|---|---|---|---|---|
| median (range) - g/dL | | | | |
| Normal karyotypeb | 12 | (57) | 1 | (100) |

Values are shown n (%) unless otherwise indicated.
^{a}Number of transfusions in 8 weeks during the 16 weeks prestudy.
^{b}Missing karyotype data for 2 patients.

Patients received imetelstat for a median of 101.1 weeks (range, 75.1-163.9 weeks) and a median of 24 cycles (range, 18-41 cycles). The median duration of TI for imetelstat ≥1-year TI responders was 123 weeks (95% CI, 80.4 to not evaluable); the median increase in hemoglobin during the longest TI interval was 5.18 g/dL (range, 2.67-13.76 g/dL) for the imetelstat group vs 1.67 g/dL for the placebo patient. After a median follow-up of 125 weeks, none of the patients with ≥1 year TI on either arm progressed to acute myeloid leukemia (AML). Of the patients receiving imetelstat, 7 had an abnormal karyotype at baseline, of which 6 had reduction in the cytogenetic abnormal clones (4 with cytogenetic complete response and 2 with cytogenetic partial response by independent review committee). Eighteen patients had mutation data available, all with SF3B1 mutations present at baseline, and multiple patients concurrently had TET2, DNMT3A, ASXL1, or JAK2 mutations. The maximal reduction ranged from −6% to −100% in SF3B1 VAF in these patients, and 13 of 18 (72.2%) achieved ≥50% VAF reduction, including 7 with complete elimination of the VAF.

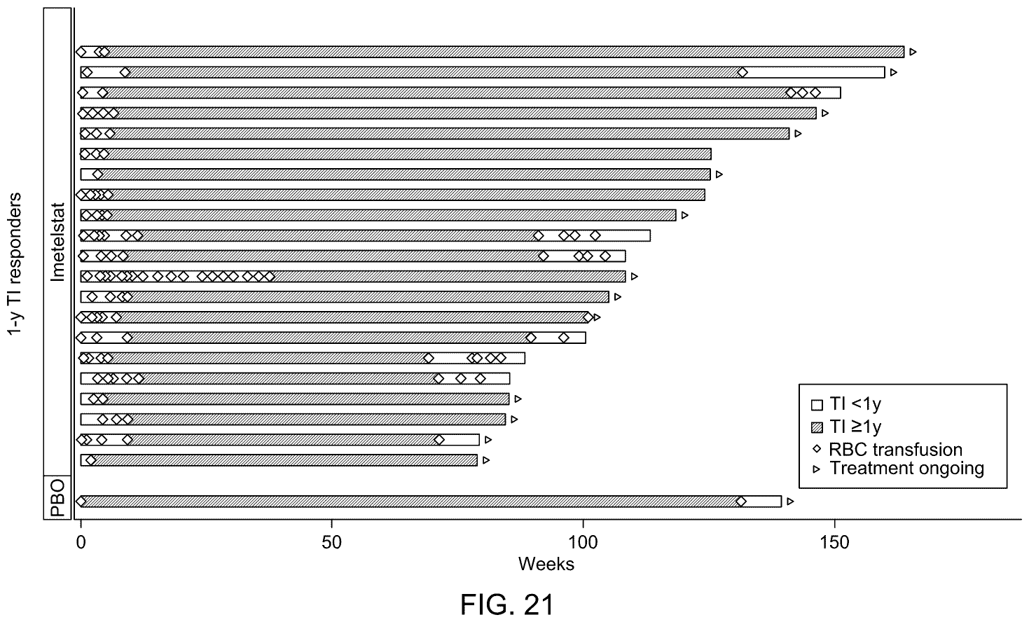

Reduction in other concurrent mutations was also observed in these patients. Safety was consistent with that previously reported; most frequent adverse events were reversible grade 3 or 4 thrombocytopenia and neutropenia. At the time of data cutoff, 13 patients receiving imetelstat and the patient receiving placebo were ongoing (FIG. 21). Of the 8 who discontinued treatment, 7 had loss of response, and 1 discontinuation was due to adverse event. Analyses for progression-free and overall survival were not evaluable as of this cutoff date due to insufficient follow-up.

Thus, treatment with imetelstat resulted in ≥1-year sustained, continuous TI in 17.8% of patients in the IMerge P3 trial. In this ESA-R/R/ineligible population with a high prior transfusion burden, a reduction to 0 RBC transfusions for ≥1 year was associated with decreased risk of transfusion associated complications including end organ dysfunction from iron overload—and decreased demand on health care resources. Furthermore, durable TI and meaningful reductions in mutational burden suggest imetelstat may have disease-modifying activity.

### Example 6: The Impact of MDS-Associated Mutations on Clinical Efficacy of Imetelstat

To evaluate the impact of MDS-associated mutations on clinical efficacy of imetelstat, next-generation sequencing was performed of a panel of 36 genes that are recurrently mutated in MDS. For DNA sequencing, samples from peripheral blood collected at study entry was used. Further analysis of TI responses to imetelstat was performed across different mutation subgroups, defined based on genes involved in different biological functions, including the splicing process, epigenetic modifiers, transcription regulation, and receptors/kinases.

Baseline mutation data were available in 165 of 178 patients (imetelstat, n=110; placebo, n=55; 93.2% and 91.7% of total in each group, respectively). Of patients with mutation data, 161 (97.6%) had ≥1 mutation detected, among whom, 75 (70.1%), 33 (30.8%), and 9 (8.4%) patients in the imetelstat group and 38 (70.4%), 15 (27.8%), and 7 (13%) patients in the placebo group had >1, >2, and >3 mutations, respectively. The ≥8-week TI rates in the imetelstat vs. placebo groups were 42.7% vs 15.8% (p=0.006) for patients with >1 mutation, 45.5% vs 6.7% (p=0.012) for patients with >2 mutations, and 55.6% vs. 14.3% (p=0.089) for patients with >3 mutations, respectively. The ≥24-week TI rates were 26.7% vs. 2.6% (p=0.003), 33.3% vs. 0% (p=0.014), and 33.3% vs. 0% (p=0.117), respectively.

In patients with mutations associated with poor prognosis (TP53, ETV6, RUNX1, ASXL1, or EZH2), 31.8% and 9.1% of patients in the imetelstat group achieved ≥8-week and >24-week TI vs. 0 of those in the placebo group. TP53 mutations were detected in 2 patients in each group; both patients in the imetelstat group and none in the placebo group had ≥8-week TI. Among patients with ASXL1 mutations, 5 of 18 patients (27.8%) in the imetelstat and 0 of 6 (0) in placebo group had ≥8-week TI. Among patients with ETV6 mutations, 1 of 2 (50%) in the imetelstat group and 0 of 1 patients in the placebo group had ≥8-week TI. Two patients in each group had RUNX1 mutations; none achieved TI.

Additional analysis of TI-responses was performed across four mutational subgroups defined based on the genes involved in different biological functions, including: RNA spliceosome (e.g., SF3B1, U2AF1, SRSF2, and ZRSR2), epigenetic modifiers (e.g., TET2, DNMT3A, IDH1, IDH2, ASXL1, and EZH2), transcription regulation (e.g., RUNX1, BCOR, ETV6, SETBP1, GATA2, CEBPA, PHF6, NPM1, and STAT3), and receptors/kinases (e.g., CSF3R, FLT3, JAK2, KRAS, KIT, MPL, NRAS, and PTPN11). Imetelstat ≥8-week TI rates were 43.8%, 37.7%, 40.0%, and 80.0% for patients harboring mutated genes in the RNA spliceosome, epigenetic modifiers, transcription regulation, and receptors/kinases, respectively. The ≥24-week TI was 30.2%, 27.5%, 20.0%, and 80.0%, respectively. The most frequently mutated gene was SF3B1 (125 of 165; 75.8%). The ≥8-week and >24-week TI rates were 48.8% vs. 16.3% (p=0.001) and 35.4% vs. 2.3% (p<0.001) with imetelstat vs placebo. In the imetelstat group, ≥8-week TI was achieved in patients with different spectrum of SF3B1 hot-spot mutations: 2 of 8 patients (25.0%) with E622D, 4 of 7 (57.1%) with R625C/L/G, 7 of 12 (58.3%) with H662Q/N/D/Y, 2 of 2 (100%) with T663P, 2 of 6 (33.3%) with K666R/T/Q/N, 18 of 41 (43.9%) with K700E, 2 of 2 (100%) with A744P, and 1 of 2 patients (50.0%) with E783K. Durable ≥24-week TI was also observed in patients with these hot-spot mutations.

Other genes with mutation frequency>10% were TET2 (32.7%), DNMT3A (17.0%), ASXL1 (14.5%), and CUX1 (12.7%). The ≥8-week TI rates in the imetelstat group vs. placebo group were 50% vs 21.4% for TET2 mutations; 31.6% vs. 22.2% for DNMT3A mutations; 27.8% vs. 0 for ASXL1 mutations; 35.7% vs. 14.3% for CUX1 mutations (FIG. 22). The ≥24-week TI rates were 40% vs. 0, 26.3% vs. 11.1%, 11.1% vs. 0, and 14.3% vs. 0, respectively, for those mutations (FIG. 22).

Thus, higher RBC-TI rates were observed in patients with various baseline mutational profiles treated with imetelstat compared with placebo in IMerge. TI responses in patients receiving imetelstat occurred regardless of the presence of mutations associated with poor prognosis or the number of mutations. Imetelstat showed comparable TI rates across different molecularly defined subgroups, suggesting that clinical benefit of imetelstat in patients with LR-MDS is independent of the underlying molecular pattern.

Thus, in patients with MDS, SF3B1 (involved in RNA splicing), TET2, DNMT3A, and ASXL1 (involved in epigenetic regulation) are commonly mutated genes, and quantification of these and other gene mutations indicates disease burden and guides disease management.

In particular, a mechanistic link between the high prevalence of the SF3B1 mutation in MDS with ring sideroblasts has been established. Imetelstat is a first-in-class, direct and competitive inhibitor of telomerase activity that specifically targets dysplastic clones, enabling recovery of effective hematopoiesis.

As described herein, in the IMerge phase 3 clinical trial of patients with TI-dependent non-del(5q) LR-MDS relapsed/refractory to/ineligible for ESAs, imetelstat showed higher TI for ≥8 weeks, ≥24 weeks, and >1 year (40%, 28%, and 18%) than placebo (15%, 3%, and 2%).

Additionally, compared with placebo, treatment with imetelstat improved cytogenetic response rate, had a higher rate of patients achieving ≥50% reduction in bone marrow RS cells (41% vs 10%) and greater VAF reduction of the SF3B1, TET2, DNMT3A, and ASXL1 genes that correlated with clinical end points of RBC-TI response, longer duration of TI, and increase in hemoglobin levels.

### Example 7: Imetelstat Treatment Resulted in Sustained Reduction of Mutation Burden Over Time

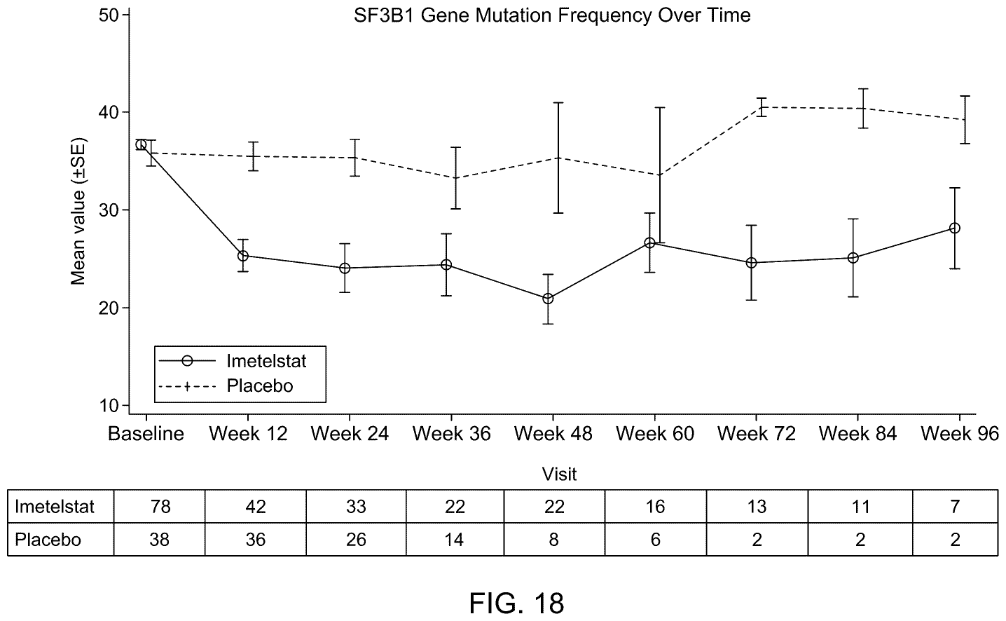

Somatic point mutations are common in MDS. Some mutations correlate strongly with features of the clinical phenotype, including specific cytopenias, blast percentage, cytogenetic abnormalities, and overall survival of patients with MDS. Decrease in mutation VAF would suggest a reduction in malignant clonal and disease burden. As described herein, in the IMerge phase 3 clinical trial of patients, the imetelstat group had sustained reduction of SF3B1 VAF over time compared to the placebo group (FIG. 18).

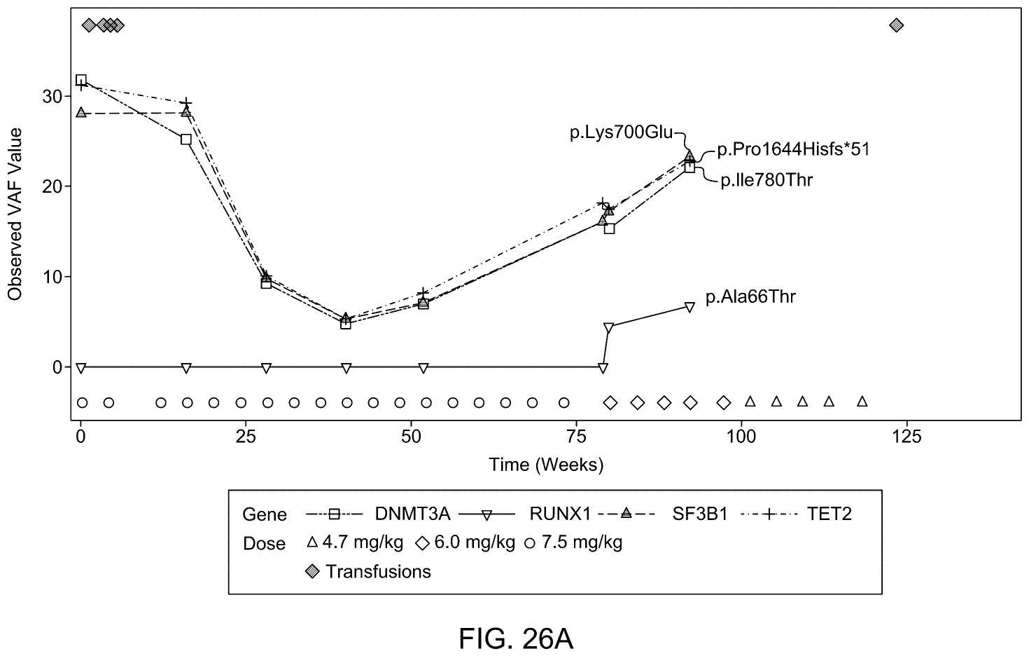

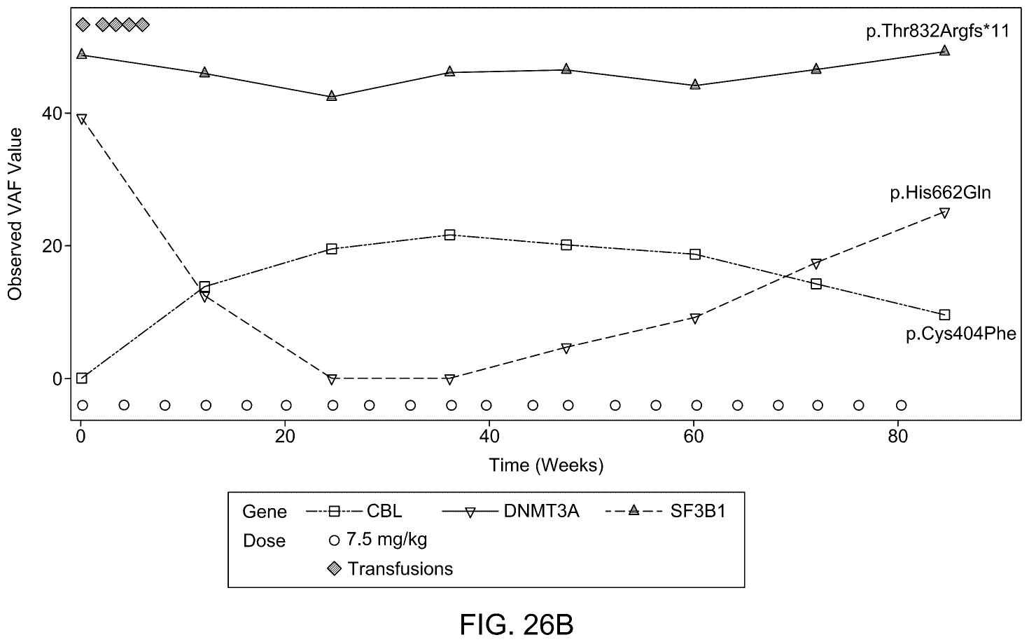

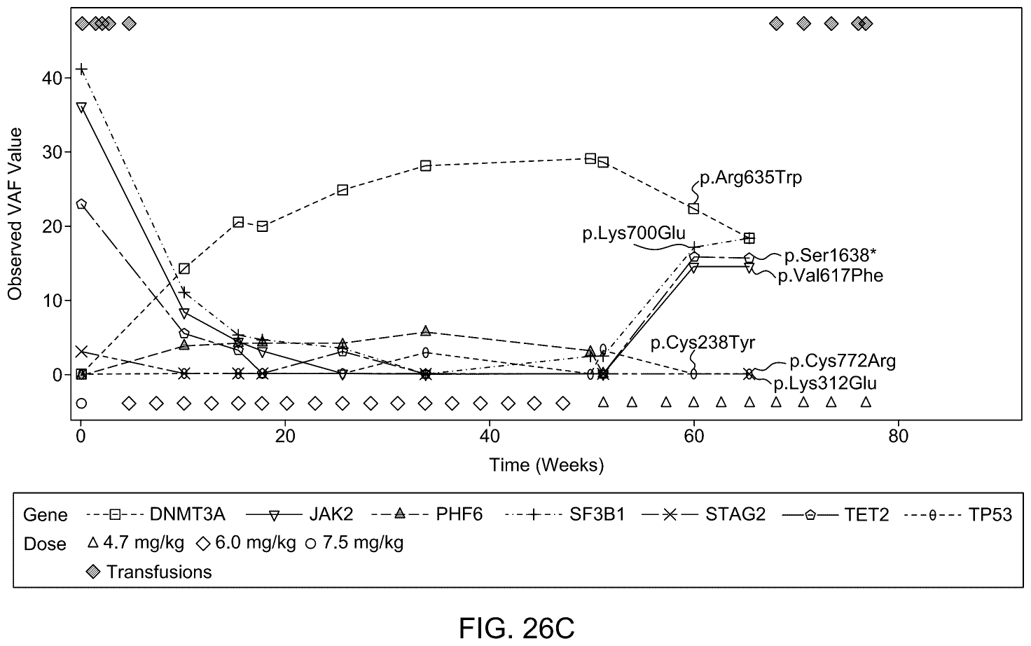

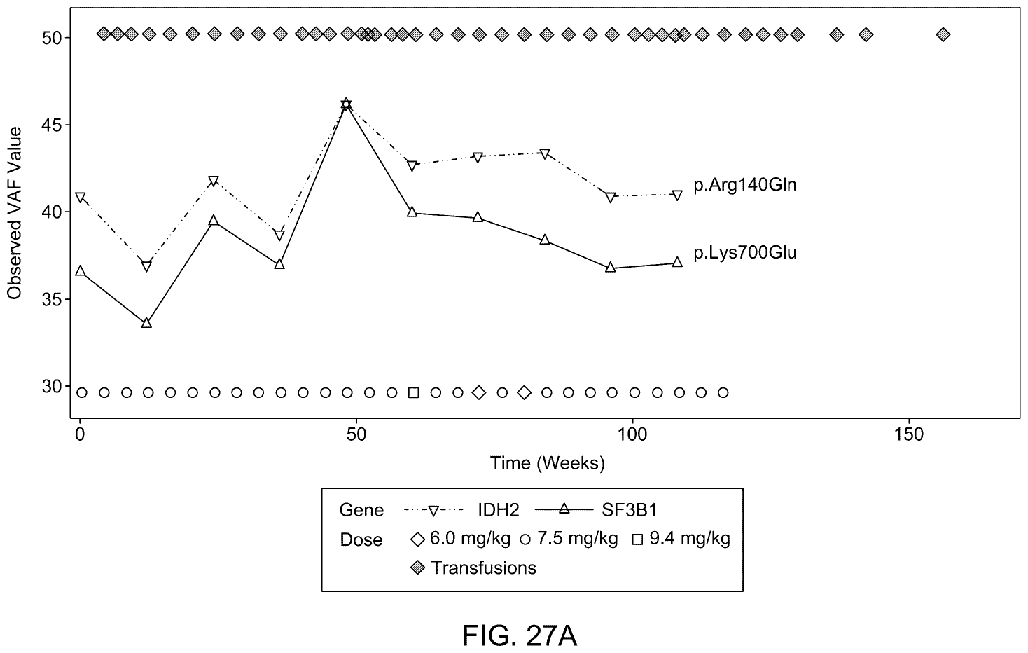

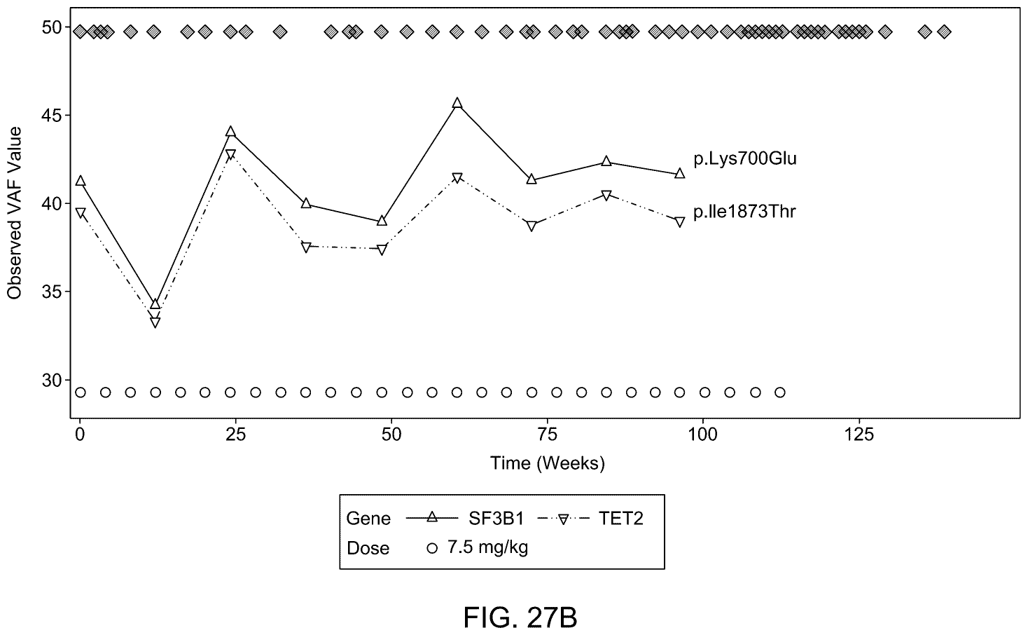

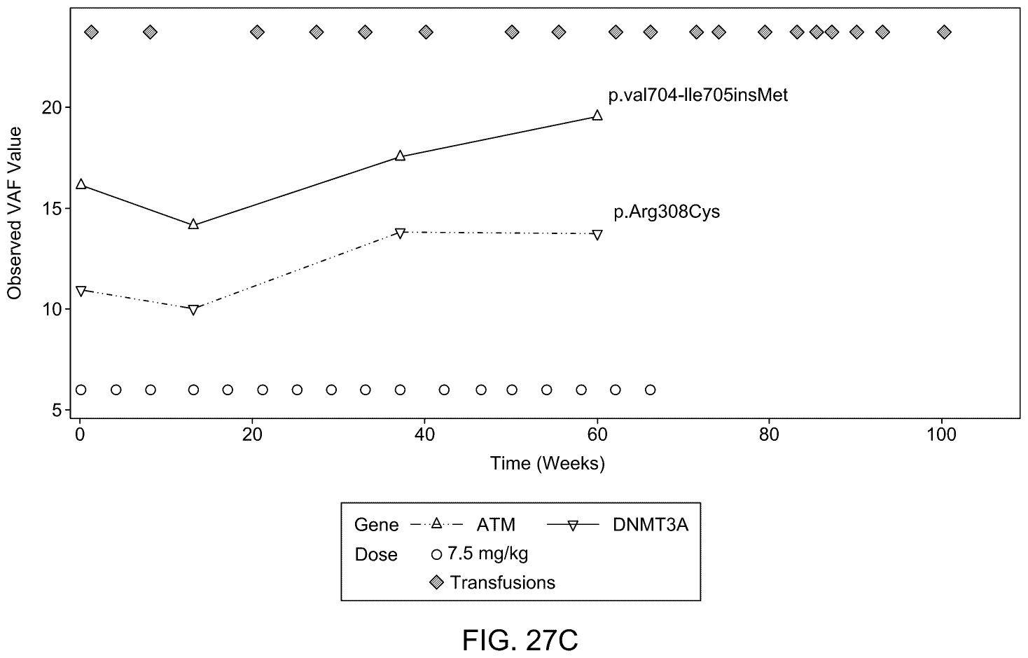

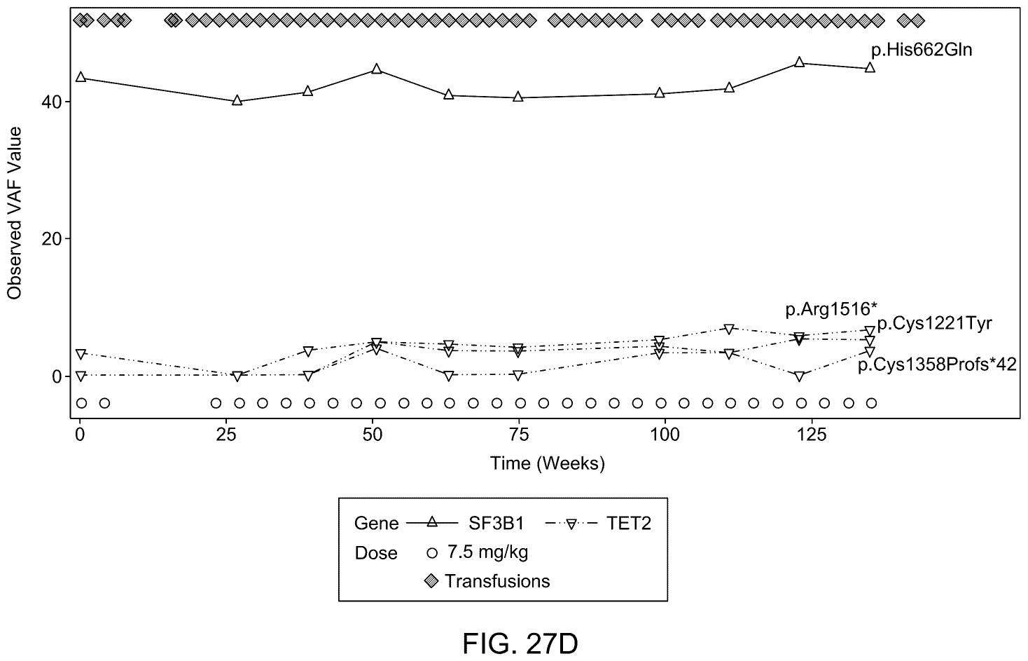

As shown as examples in FIG. 26A-C, 3 patients treated with imetelstat achieved >1 year TI, which is correlated with sustained decrease in VAF or elimination of mutation clones in multiple genes (e.g., SF3B1, DNMTA3, TET2, JAK2) during the treatment. The time with increase of VAF and emerging of new mutations was concurrent or preceded the recurrence of transfusion dependence. Placebo treated patients had very limited impact on mutation VAF over time (FIG. 27A-C).

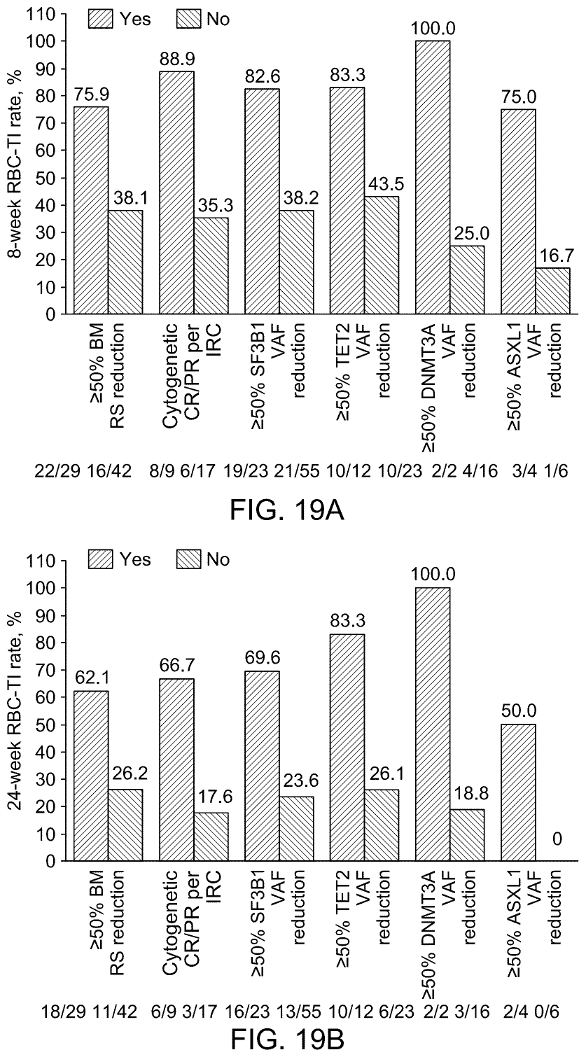

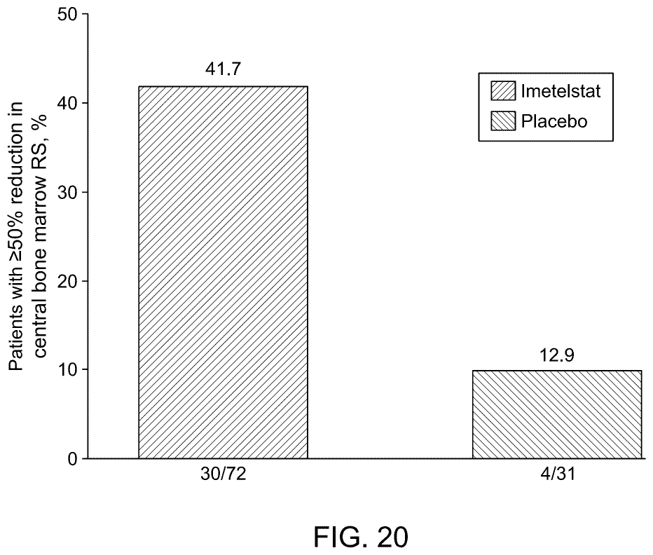

In addition, the reduction of VAF in multiple genes by imetelstat correlated with TI response (FIGS. 19A and 19B), longer RBC-TI duration (FIG. 12B, FIGS. 17A and 17B), and increase in Hgb levels (FIG. 12c, FIGS. 17C and 17D), suggesting that imetelstat may alter the underlying biology of LR-MDS and potentially modify the disease by reducing or eliminating malignant clones and improving ineffective erythropoiesis.

Notwithstanding the set of claims provided herein, the disclosure provides the clauses listed below:

Clause 1. A method of identifying a subject with myelodysplastic syndrome (MDS) for treatment with a telomerase inhibitor, the method comprising:
assessing variant allele frequency (VAF) for one or more genes selected from the group consisting of SF3B1, TET2, DNMT3A and ASXL1 in a biological sample obtained from the subject after administration of the telomerase inhibitor; and
comparing the VAF for the one or more genes to a baseline VAF for the one or more genes prior to administration of the telomerase inhibitor,
wherein a 25% or more reduction in the VAF for the one or more genes in the biological sample identifies a subject who has an increased likelihood of benefiting from treatment with the telomerase inhibitor.

Clause 2. The method of Clause 1, further comprising continuing the treatment with the telomerase inhibitor if the biological sample obtained from the patient has a 25% or more reduction in the VAF for the one or more genes.

Clause 3. The method of any one of Clauses 1-2, wherein the subject is diagnosed as having trisomy 8.

Clause 4. The method of Clause 3, wherein the subject is diagnosed as having trisomy 8 with mosaicism.

Clause 5. The method of any one of Clauses 3-4, further comprising diagnosing the subject with trisomy 8.

Clause 6. A method of treating myelodysplastic syndrome (MDS), the method comprising:
administering to a subject in need thereof an effective amount of a telomerase inhibitor; and
assessing variant allele frequency (VAF) for one or more genes selected from the group consisting of SF3B1, TET2, DNMT3A and ASXL1 in a biological sample obtained from the subject after administration of the telomerase inhibitor.

Clause 7. The method of Clause 6, wherein the VAF for the one or more genes is reduced by 25% or more relative to a baseline VAF for the one or more genes prior to administration of the telomerase inhibitor.

Clause 8. The method of Clause 6 or 7, further comprising altering the dosage of the telomerase inhibitor, the frequency of dosing, or the course of therapy administered to the subject based on the assessing.

Clause 9. The method of any one of Clauses 6-8, wherein the subject is diagnosed as having trisomy 8.

Clause 10. The method of Clause 9, wherein the subject is diagnosed as having trisomy 8 with mosaicism.

Clause 11. The method of any one of Clauses 9-10, further comprising diagnosing the subject with trisomy 8.

Clause 12. A method of monitoring therapeutic efficacy in a subject with myelodysplastic syndrome (MDS), the method comprising:
assessing variant allele frequency (VAF) for one or more genes selected from the group consisting of SF3B1, TET2, DNMT3A and ASXL1 in a biological sample obtained from the subject after administration of a telomerase inhibitor; and
comparing the VAF for the one or more genes to a baseline VAF for the one or more genes prior to administration of the telomerase inhibitor,
wherein a 25% or more reduction in the VAF for the one or more genes in the biological sample identifies a subject who has an increased likelihood of benefiting from treatment with the telomerase inhibitor.

Clause 13. The method of any one of Clauses 1-12, wherein the subject is naive to treatment with an agent selected from a hypomethylating agent (HMA), lenalidomide, and combination thereof.

Clause 14. The method of any one of Clauses 1-13, wherein the MDS is relapsed or refractory MDS.

Clause 15. The method of any one of Clauses 1-14, wherein the MDS is MDS relapsed or refractory to erythropoiesis-stimulating agent (ESA).

Clause 16. The method of any one of Clauses 1-15, wherein the subject is classified as a low or intermediate-1 IPSS risk MDS subject.

Clause 17. The method of any one of Clauses 1-16, wherein the subject is transfusion dependent.

Clause 18. The method of Clause 17, wherein the transfusion dependent subject has a transfusion requirement of about 4 units or more during the 8 weeks prior to the administration of the telomerase inhibitor.

Clause 19. The method of any one of Clauses 1-18, wherein the subject is a non-del5q human patient.

Clause 20. The method of any one of Clauses 1-19, wherein the subject is naive to treatment with lenalidomide.

Clause 21. The method of any one of Clauses 1-19, wherein the subject is naive to treatment with HMA selected from decitabine and azacitidine.

Clause 22. The method of any one of Clauses 1-21, wherein the telomerase inhibitor is imetelstat.

Clause 23. The method of Clause 22, wherein the imetelstat is imetelstat sodium.

Clause 24. The method of Clause 23, wherein the telomerase inhibitor is imetelstat and is administered for 1, 2, 3, 4, 5, 6, 7, 8 or more than 8 dosage cycles, each cycle comprising:
(a) intravenous administration of 7-10 mg/kg imetelstat once every four weeks;
(b) intravenous administration of 7-10 mg/kg imetelstat once weekly for four weeks;
(c) intravenous administration of 2.5-10 mg/kg imetelstat once every three weeks; or
(d) intravenous administration of 0.5-9.4 mg/kg imetelstat once every four weeks.

Clause 25. The method of Clause 24, wherein each dosage cycle comprises intravenous administration of 7-10 mg/kg imetelstat once every four weeks.

Clause 26. The method of Clause 24, wherein the telomerase inhibitor is imetelstat sodium and is administered for 1, 2, 3, 4, 5, 6, 7, 8 or more than 8 dosage cycles, each cycle comprising:
(a) intravenous administration of 7-10 mg/kg imetelstat sodium once every four weeks;
(b) intravenous administration of 7-10 mg/kg imetelstat sodium once weekly for four weeks;
(c) intravenous administration of 2.5-10 mg/kg imetelstat sodium once every three weeks; or
(d) intravenous administration of 0.5-9.4 mg/kg imetelstat sodium once every four weeks.

Clause 27. The method of Clause 26, wherein each dosage cycle comprises intravenous administration of 4.5-11.7 mg/kg imetelstat sodium once every four weeks.

Clause 28. The method of Clause 26, wherein each dosage cycle comprises intravenous administration of 7.5 mg/kg imetelstat sodium once every four weeks.

Clause 29. A method of identifying a subject with myelodysplastic syndrome (MDS) for treatment with a telomerase inhibitor, the method comprising:
assessing variant allele frequency (VAF) for one or more genes selected from the group consisting of SF3B1, TET2, DNMT3A, CUX1 and ASXL1 in a biological sample obtained from the subject after administration of the telomerase inhibitor; and
comparing the VAF for the one or more genes to a baseline VAF for the one or more genes prior to administration of the telomerase inhibitor,
wherein a 25% or more reduction in the VAF for the one or more genes in the biological sample identifies a subject who has an increased likelihood of benefiting from treatment with the telomerase inhibitor.

Clause 30. The method of Clause 29, further comprising continuing the treatment with the telomerase inhibitor if the biological sample obtained from the patient has a 25% or more reduction in the VAF for the one or more genes.

Clause 31. The method of any one of Clauses 29-30, wherein the subject is diagnosed as having trisomy 8.

Clause 32. The method of Clause 31, wherein the subject is diagnosed as having trisomy 8 with mosaicism.

Clause 33. The method of any one of Clauses 31-32, further comprising diagnosing the subject with trisomy 8.

Clause 34. A method of treating myelodysplastic syndrome (MDS), the method comprising:
administering to a subject in need thereof an effective amount of a telomerase inhibitor; and
assessing variant allele frequency (VAF) for one or more genes selected from the group consisting of SF3B1, TET2, DNMT3A, CUX1 and ASXL1 in a biological sample obtained from the subject after administration of the telomerase inhibitor.

Clause 35. The method of Clause 34, wherein the VAF for the one or more genes is reduced by 25% or more relative to a baseline VAF for the one or more genes prior to administration of the telomerase inhibitor.

Clause 36. The method of Clause 34 or 35, further comprising altering the dosage of the telomerase inhibitor, the frequency of dosing, or the course of therapy administered to the subject based on the assessing.

Clause 37. The method of any one of Clauses 34-36, wherein the subject is diagnosed as having trisomy 8.

Clause 38. The method of Clause 37, wherein the subject is diagnosed as having trisomy 8 with mosaicism.

Clause 39. The method of any one of Clauses 37-38, further comprising diagnosing the subject with trisomy 8.

Clause 40. A method of monitoring therapeutic efficacy in a subject with myelodysplastic syndrome (MDS), the method comprising:
assessing variant allele frequency (VAF) for one or more genes selected from the group consisting of SF3B1, TET2, DNMT3A, CUX1 and ASXL1 in a biological sample obtained from the subject after administration of a telomerase inhibitor; and
comparing the VAF for the one or more genes to a baseline VAF for the one or more genes prior to administration of the telomerase inhibitor,
wherein a 25% or more reduction in the VAF for the one or more genes in the biological sample identifies a subject who has an increased likelihood of benefiting from treatment with the telomerase inhibitor.

Clause 41. The method of any one of Clauses 29-40, wherein the subject is naive to treatment with an agent selected from a hypomethylating agent (HMA), lenalidomide, and combination thereof.

Clause 42. The method of any one of Clauses 29-41, wherein the MDS is relapsed or refractory MDS.

Clause 43. The method of any one of Clauses 29-42, wherein the MDS is MDS relapsed or refractory to erythropoiesis-stimulating agent (ESA).

Clause 44. The method of any one of Clauses 29-43, wherein the subject is classified as a low or intermediate-1 IPSS risk MDS subject.

Clause 45. The method of any one of Clauses 29-44, wherein the subject is transfusion dependent.

Clause 46. The method of Clause 45, wherein the transfusion dependent subject has a transfusion requirement of about 4 units or more during the 8 weeks prior to the administration of the telomerase inhibitor.

Clause 47. The method of any one of Clauses 29-46, wherein the subject is a non-del5q human patient.

Clause 48. The method of any one of Clauses 29-47, wherein the subject is naive to treatment with lenalidomide.

Clause 49. The method of any one of Clauses 29-48, wherein the subject is naive to treatment with HMA selected from decitabine and azacitidine.

Clause 50. The method of any one of Clauses 29-49, wherein the telomerase inhibitor is imetelstat.

Clause 51. The method of Clause 50, wherein the imetelstat is imetelstat sodium.

Clause 52. The method of Clause 51, wherein the telomerase inhibitor is imetelstat and is administered for 1, 2, 3, 4, 5, 6, 7, 8 or more than 8 dosage cycles, each cycle comprising:
(e) intravenous administration of 7-10 mg/kg imetelstat once every four weeks;
(f) intravenous administration of 7-10 mg/kg imetelstat once weekly for four weeks;
(g) intravenous administration of 2.5-10 mg/kg imetelstat once every three weeks; or
(h) intravenous administration of 0.5-9.4 mg/kg imetelstat once every four weeks.

Clause 53. The method of Clause 52, wherein each dosage cycle comprises intravenous administration of 7-10 mg/kg imetelstat once every four weeks.

Clause 54. The method of Clause 52, wherein the telomerase inhibitor is imetelstat sodium and is administered for 1, 2, 3, 4, 5, 6, 7, 8 or more than 8 dosage cycles, each cycle comprising:
(e) intravenous administration of 7-10 mg/kg imetelstat sodium once every four weeks;
(f) intravenous administration of 7-10 mg/kg imetelstat sodium once weekly for four weeks;
(g) intravenous administration of 2.5-10 mg/kg imetelstat sodium once every three weeks; or
(h) intravenous administration of 0.5-9.4 mg/kg imetelstat sodium once every four weeks.

Clause 55. The method of Clause 54, wherein each dosage cycle comprises intravenous administration of 4.5-11.7 mg/kg imetelstat sodium once every four weeks.

Clause 56. The method of Clause 54, wherein each dosage cycle comprises intravenous administration of 7.5 mg/kg imetelstat sodium once every four weeks.

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1-56. (canceled)

57. A method of treating MDS in a subject by administering a telomerase inhibitor, wherein the subject received a first administration of the telomerase inhibitor and after the first administration of the telomerase inhibitor, 25% or more reduction in the variant allele frequency (VAF) was observed for one or more of the following genes: SF3B1, TET2, DNMT3A, CUX1, and ASXL1.

58. The method of claim 57, wherein 25% or more reduction in the VAF was observed for one or more of the following genes: SF3B1, TET2, DNMT3A, and ASXL1.

59. The method of claim 57, comprising altering the dosage of the telomerase inhibitor, the frequency of dosing, or the course of therapy administered to the subject based on the observed VAF for the one or more genes after the first administration of the telomerase inhibitor as compared to the VAF for the one or more genes before the subject received the first administration of the telomerase inhibitor.

60. The method of claim 57, wherein the subject is naïve to treatment with an agent selected from a hypomethylating agent (HMA), lenalidomide, and combination thereof.

61. The method of claim 57, wherein the MDS is MDS relapsed or refractory to erythropoiesis-stimulating agent (ESA).

62. The method of claim 57, wherein the subject is transfusion dependent.

63. The method of claim 57, wherein the telomerase inhibitor is imetelstat.

64. The method of claim 57, wherein the imetelstat is imetelstat sodium.

65. A method of treating MDS in a subject, the method comprising first administering to the subject a telomerase inhibitor and continuing administering to the subject the telomerase inhibitor if, after the first administering of the telomerase inhibitor to the subject, 25% or more reduction in the variant allele frequency (VAF) was observed for one or more of the following genes: SF3B1, TET2, DNMT3A, CUX1, and ASXL1.

66. The method of claim 65, comprising continuing administering to the subject the telomerase inhibitor if, after the first administering of the telomerase inhibitor to the subject, 25% or more reduction in the VAF was observed for one or more of the following genes: SF3B1, TET2, DNMT3A, and ASXL1.

67. The method of claim 65, comprising altering the dosage of the telomerase inhibitor, the frequency of dosing, or the course of therapy administered to the subject based on the observed VAF for the one or more genes after the first administering of the telomerase inhibitor as compared to the VAF for the one or more genes before the first administering of the telomerase inhibitor.

68. The method of claim 65, wherein the subject is naïve to treatment with an agent selected from a hypomethylating agent (HMA), lenalidomide, and combination thereof.

69. The method of claim 65, wherein the MDS is MDS relapsed or refractory to erythropoiesis-stimulating agent (ESA).

70. The method of claim 65, wherein the subject is transfusion dependent.

71. The method of claim 65, wherein the telomerase inhibitor is imetelstat.

72. The method of claim 57 wherein the imetelstat is imetelstat sodium.

73. A method of treating a subject with myelodysplastic syndrome (MDS), the method comprising:
assessing variant allele frequency (VAF) for one or more genes selected from the group consisting of SF3B1, TET2, DNMT3A, CUX1, and ASXL1 in a biological sample obtained from a subject with MDS after a first administration of a telomerase inhibitor; and
comparing the VAF for the one or more genes to a baseline VAF for the one or more genes prior to administration of the telomerase inhibitor,
identifying the subject as having an increased likelihood of benefitting from treatment with the telomerase inhibitor if a 25% or more reduction in the VAF is observed for the one or more genes,
continuing administering the telomerase inhibitor to the subject identified as having an increased likelihood of benefiting from treatment with the telomerase inhibitor.

74. The method of claim 73, comprising:
assessing VAF for one or more genes selected from the group consisting of SF3B1, TET2, DNMT3A, and ASXL1 in a biological sample obtained from a subject with MDS after the first administration of a telomerase inhibitor; and
comparing the VAF for the one or more genes to a baseline VAF for the one or more genes prior to administration of the telomerase inhibitor,
identifying the subject as having an increased likelihood of benefitting from treatment with the telomerase inhibitor if a 25% or more reduction in the VAF is observed for the one or more genes,
continuing administering the telomerase inhibitor to the subject identified as having an increased likelihood of benefiting from treatment with the telomerase inhibitor.

75. The method of claim 73, comprising altering the dosage of the telomerase inhibitor, the frequency of dosing, or the course of therapy administered to the subject based on the observed VAF for the one or more genes after the first administration of the telomerase inhibitor as compared to the VAF for the one or more genes before the subject received the first administration of the telomerase inhibitor.

76. The method of claim 73, wherein the subject is naïve to treatment with an agent selected from a hypomethylating agent (HMA), lenalidomide, and combination thereof.

77. The method of claim 73, wherein the MDS is MDS relapsed or refractory to erythropoiesis-stimulating agent (ESA).

78. The method of claim 73, wherein the subject is transfusion dependent.

79. The method of claim 73, wherein the telomerase inhibitor is imetelstat.

80. The method of claim 73, wherein the imetelstat is imetelstat sodium.


SEQUENCE LISTING (1 sequence)

SEQ ID NO: 1 - DNA, 13 bp, synthetic construct
  source 1..13: mol_type = other DNA
tagggttagacaa